(19) United States
(12) Patent Application Publication
(10) Pub. No.: US 2024/0011048 A1
(43) Pub. Date: Jan. 11, 2024

(54) METHODS AND COMPOSITIONS FOR INHIBITING EXCESS NUCLEIC ACID PRECIPITATION

(71) Applicant: PFIZER INC., New York, NY (US)

(72) Inventors: Larry Dean DETERMAN, Chesterfield, MO (US); Nathaniel A. JENKINS, Chesterfield, MO (US); Daniel KOBACK, Morrisville, NC (US); Delaney Kate KOLICH, Morrisville, NC (US); Paul B. LANTER, Chesterfield, MO (US); Seyed Pouria MOTEVALIAN, Waban, MA (US); Kathryn C. OLSON, Morrisville, NC (US); Jeffrey William PAVLICEK, Morrisville, NC (US); Austin Stenhen TRITT, Morrisville, NC (US); Vincent WINGATE, Morrisville, NC (US)

(21) Appl. No.: 18/255,870

(22) PCT Filed: Dec. 17, 2021
(86) PCT No.: PCT/IB2021/061952

§ 371 Date: Jun. 4, 2023

Related U.S. Application Data
(60) Provisional application No. 63/199,367, filed on Dec. 21, 2020.
(60) Provisional application No. 63/264,997, filed on Dec. 6, 2021.

(51) Int. Cl. C12N 15/86; C12M 1/02

(52) U.S. Cl. CPC C12N 15/86; C12M 27/00; C12N 2750/14143; C12N 2750/14151

(57) ABSTRACT

The present disclosure provides improved methods and systems for transfecting host cells with nucleic acids, such as plasmid DNA, for purposes of efficiently producing biological products, such as AAV vectors, at large scale.

### CROSS REFERENCE TO RELATED APPLICATIONS

This application claims the benefit of U.S. Provisional Application No. 63/199,367, filed Dec. 21, 2020, and U.S. Provisional Application No. 63/264,997, filed Dec. 6, 2021, the contents of each of which are hereby incorporated by reference in their entirety.

### FIELD OF THE INVENTION

The present disclosure relates generally to the field of cell transfection with nucleic acids, and more specifically to improved methods and systems for preparing and delivering transfection cocktail to cells in a manner that maintains high transfection efficiency.

### BACKGROUND OF THE INVENTION

Production of many products in the biotechnology industry involves introducing genetic material, such as DNA plasmids, into host cells that serve as living factories whose metabolic and biosynthetic activities are directed by genetic information embodied in the material. This information can encode proteins with therapeutic or industrial utility, examples of which include monoclonal antibodies, enzymes, clotting factors and protein components of gene therapy vectors. The information can also include nucleotide sequences that are not expressed as proteins in the host cells, but are instead transcribed or replicated and combined with other components, an example of which are modified genomes derived from adeno-associated virus (AAV) which, when packaged with AAV structural proteins expressed in the same cells, can form recombinant AAV vectors useful for gene therapy.

Introduction of genetic material into cells can be accomplished in many ways. For example, genetic material can be introduced using viral vectors, or physically, such as by gene gun or electroporation. But one of the most common transfection methods employs chemical compounds, known as transfection reagents, that complex with and condense nucleic acids to form tiny particles, which can be taken up by cells and be acted upon by cellular machinery to guide replication, transcription or protein expression. In these methods, transfection reagent is typically mixed in a solution with the nucleic acid of interest, forming a so-called transfection cocktail.

Many different chemical compounds can serve as transfection reagents, examples of which include calcium phosphate, artificial liposomes, and cationic polymers, such as diethylaminoethyl (DEAE)-dextran and polyethylenimine (PEI). In general, chemically-based transfection reagents are rich in positive charges that can shield the negatively charged phosphate backbone of DNA or RNA, thereby facilitating entry of the particles of complexed transfection reagent and nucleic acid into cells through cell membranes, which often have a negative charge.

Many variables can influence transfection efficiency in terms of the proportion of genetic material that is actually taken up by host cells, reaches host cell nuclei, or is competent to guide cellular behavior. For example, it is well known that the calcium phosphate method is highly sensitive to the pH of the transfection cocktail, so this variable must be carefully controlled to optimize transfection efficiency and therefore production by host cells of a desired product produced under the direction of the genetic information in the transfected nucleic acids. Another variable that impacts efficiency of different transfection reagents is the amount of time that transfection cocktail is incubated before it is added to the cells to be transfected. Extended incubation of transfection cocktails containing calcium chloride or PEI, for example, have been reported to reduce transfection efficiency, possibly because longer incubation results in larger particles of complexed transfection reagent and nucleic acid (Jordan, M, et al., Transfecting mammalian cells: optimization of critical parameters affecting calcium-phosphate precipitate formation, Nuc. Acids Res. 24(4):596-601 (1996); Sang, Y, et al., Salt ions and related parameters affect PEI—DNA particle size and transfection efficiency in Chinese hamster ovary cells, Cytotechnology 67:67-74 (2015).

The inverse relationship between transfection cocktail incubation time and transfection efficiency is not a significant problem when transfections are performed at relatively small scale. After preparing a transfection cocktail of limited volume, it can be added to cells relatively quickly, such as by pumping or pouring, before particle size has increased to the point where it significantly reduces efficiency. At industrial scale, however, where tens to hundreds of liters of transfection cocktail may be needed to transfect hundreds to thousands of liters of cells in culture, the ensuing delay between preparing the cocktail and adding it to the cells at a rate that does not raise the local concentration to toxic levels can be significant, with a concomitant reduction in transfection efficiency. For some products, such as gene therapy vectors, which by their nature require numerous complex steps to make and purify, low transfection efficiency at the beginning of the overall manufacturing process will inevitably reduce yields and increase costs, potentially rendering a promising therapeutic agent uneconomic to produce.

Accordingly, there remains a need in the art for methods and systems to prepare relatively large volumes of transfection cocktail and deliver it to cells in culture over relatively short periods of time so as to maintain high levels of transfection efficiency.

### SUMMARY OF THE INVENTION

The present disclosure solves these and other problems in the art by providing novel methods and systems for preparing and delivering even large volumes of transfection cocktail to cells in culture in relatively short periods of time, thereby resulting in high levels of transfection efficiency. These methods and systems, which are suitable for transfecting cells grown to high densities, can be employed to efficiently produce many different biological products in cells, including proteins as well as multi-component biological products, such as gene therapy vectors.

Those skilled in the art will recognize or be able to ascertain using no more than routine experimentation many equivalents to the specific embodiments of the invention described herein. Such equivalents are intended to be encompassed by the following enumerated embodiments (E).

E1. In a first embodiment, the disclosure provides methods of transiently transfecting cells with nucleic acid, comprising the steps of (i) preparing a transfection cocktail comprising nucleic acid and a transfection agent, and (ii) adding the transfection cocktail to a sample of cells in culture.

E2. The method of E1, wherein in some embodiments the step of preparing the transfection cocktail comprises mixing a first solution comprising the nucleic acid and a second solution comprising the transfection agent.

E3. The method of any one of E1 to E2, wherein in some embodiments the steps of preparing the transfection cocktail and adding it to the cells in culture are performed discontinuously, such as in a single bolus, or in a plurality of segmented boluses.

E4. The method of any one of E1 to E2, wherein in some embodiments the steps of preparing the transfection cocktail and adding it to the cells in culture are performed continuously.

E5. The method of any one of E1 to E4, wherein in some embodiments the time between initiating preparing the transfection cocktail and initiating adding the transfection cocktail is about, is at most, or is at least 30 minutes or less, such as 29, 28, 27, 26, 25, 24, 23, 22, 21, 20, 19, 18, 17, 16, 15, 14, 13, 12, 11, 10, 9, 8, 7, 6, 5, 4, 3, 2, or 1 minute, or less time, or a value between or range comprising any of the foregoing specifically enumerated values.

E6. The method of any one of E1 to E4, wherein in some embodiments the time between initiating preparing the transfection cocktail and initiating adding the transfection cocktail is about, is at least, or is at most 300 seconds or less, such as about 290, 280, 270, 260, 250, 240, 230, 220, 210, 200, 190, 180, 170, 160, 155, 150, 145, 140, 135, 130, 125, 120, 115, 110, 100, 95, 85, 80, 75, 70, 65, 60, 55, 50, 45, 40, 35, 30, 25, or 20 seconds, or less time, or a value between or range comprising any of the foregoing specifically enumerated values.

E7. The method of any one of E1 to E6, wherein in some embodiments the step of adding is performed for about, for at least, or for at most 2 hours or less, such as 1.5 hr, 1 hr, or about 55, 45, 40, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5 minutes, or less time, or a value between or range comprising any of the foregoing specifically enumerated values.

E8. The method of any one of E1 to E2, wherein in some embodiments (i) the time between initiating preparing the transfection cocktail and initiating adding the transfection cocktail is about, is at least, or is at most 300 seconds or less, such as 290, 280, 270, 260, 250, 240, 230, 220, 210, 200, 190, 180, 170, 160, 155, 150, 145, 140, 135, 130, 125, 120, 115, 110, 100, 95, 90, 85, 75, 70, 65, 60, 55, 50, 45, 40, 35, 30, 25, or 20 seconds, or less time, or a value between or range comprising any of the foregoing specifically enumerated values; and (ii) the step of adding is performed for about, for at least, or for at most 2 hours or less, such as 1.5 hr, 1 hr, or about 50, 45, 40, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5 minutes, or less time, or a value between or range comprising any of the foregoing specifically enumerated values.

E9. The method of any one of E1 to E2, wherein in some embodiments (i) the time between initiating preparing the transfection cocktail and initiating adding the transfection cocktail is about, is at least, or is at most 4 min, 3 min, 120 secs, 90 secs, 60 secs, or 30 secs, or a value between or range comprising any of the foregoing specifically enumerated values; and (ii) the step of adding is performed for about, for at least, or for at most 45, 40, 35, 30, 29, 28, 27, 26, 24, 23, 22, 21, 20, 19, 18, 17, 16, 15, 14, 13, 12, 11, 10, 9, 8, 7, 6, or 5 minutes, or a value between or range comprising any of the foregoing specifically enumerated values.

E10. The method of any one of E1 to E2, wherein in some embodiments (i) the time between initiating preparing the transfection cocktail and initiating adding the transfection cocktail is about, is at least, or is at most 15 to 180 secs, 30 to 120 secs, 45 to 120 secs, 60 to 120 secs, 70 to 110 secs, 80 to 110 secs, 80 to 100 secs, 85 to 95 secs, 75 to 95 secs, 65 to 95 secs, 55 to 95 secs, 50 to 95 secs, 55 to 90 secs, 55 to 85 secs, 55 to 80 secs, 55 to 75 secs, 55 to 70 secs, or 55 to 65 secs; and (ii) the step of adding is performed for about, for at least, or for at most 5 to 60 mins, 10 to 60 mins, 15 to 60 mins, 20 to 60 mins, 25 to 55 mins, 25 to 35 mins, 30 to 50 mins, 35 to 50 mins, 35 to 45 mins, 40 to 50 mins, or 45 to 50 mins.

E11. The method of any one of E1 to E2, wherein in some embodiments (i) the time between initiating preparing the transfection cocktail and initiating adding the transfection cocktail is about, is at least, or is at most 55 to 95 secs; and (ii) the step of adding is performed for about, for at least, or for at most 30 to 45 mins.

E12. The method of any one of E1 to E11, wherein in some embodiments the transfection agent is a polycationic transfection agent.

E13. The method of E12, wherein in some embodiments the polycationic transfection agent is a polyalkylenimine, such as a polyethylenimine.

E14. The method of E12, wherein in some embodiments the polycationic transfection agent is polyethylenimine (PEI).

E15. The method of E14, wherein in some embodiments the PEI is linear.

E16. The method of E14, wherein in some embodiments the PEI is branched.

E17. The method of E14, wherein in some embodiments the PEI is homogeneous.

E18. The method of E14, wherein in some embodiments the PEI is heterogeneous.

E19. The method of E14, wherein in some embodiments the PEI is fully or partially hydrolyzed, is fully or partially deacylated, is derivatized, or is conjugated.

E20. The method of E14, wherein in some embodiments the PEI is a hydrochloride salt or is a free base.

E21. The method of any one of E14 to E20, wherein in some embodiments the PEI has an average molecular weight (Mn or Mw) of about 500 Daltons (D) to 1, 10, 20, 30, 40, 50, 60, 70, 80, 90, 100, 150, 200, 250, 300, 400, 500, 600, 700, or 800 kD, or a value between or range comprising any of the foregoing specifically enumerated values.

E22. The method of any one of E14 to E21, wherein in some embodiments the PEI has an average molecular weight (Mn or Mw) of about 10 to 100 kD.

E23. The method of any one of E14 to E22, wherein in some embodiments the PEI has an average molecular weight (Mn or Mw) of about 40 kD.

E24. The method of any one of E14 to E20, wherein in some embodiments the PEI has a polydispersity index (PDI) of about, of at least, or of at most 1, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, 3.0, 3.1, 3.2, 3.3, 3.4, 3.5, 4, 5, 6, 7, 8, 9, 10, 11, 12, 13, 14, 15, 16, 17, 18, 19, 20, 25, or 30, or a value between or range comprising any of the foregoing specifically enumerated values.

E25. The method of any one of E1 to E24, wherein in some embodiments the nucleic acid is deoxyribonucleic acid (DNA).

E26. The method of E25, wherein in some embodiments the DNA is substantially purified plasmid DNA (pDNA).

E27. The method of E26, wherein in some embodiments the pDNA is propagated in a micro-organism, such as a yeast, or a bacterium.

E28. The method of any one of E26 to E27, wherein in some embodiments the pDNA is substantially supercoiled, nicked circular, or linear.

E29. The method of any one of E26 to E28, wherein in some embodiments the pDNA comprises a first type of plasmid.

E30. The method of E29, wherein in some embodiments said first type of plasmid ranges in size from about 500 base pairs (bp) to about 3 megabase pairs (Mbp).

E31. The method of any one of E26 to E28, wherein in some embodiments the pDNA comprises two or more types of plasmids, wherein the nucleotide sequence of each type is at least partly unique.

E32. The method of any one of E26 to E28, wherein in some embodiments the pDNA comprises three types of plasmids, wherein the nucleotide sequence of each type is at least partly unique.

E33. The method of any one of E29 to E32, wherein in some embodiments at least one of the types of pDNA comprises a sequence for expressing a transgene.

E34. The method of E33, wherein in some embodiments the sequence of the transgene encodes an RNA or a protein.

E35. The method of any one of E33 to E34, wherein in some embodiments the pDNA further comprises a genetic control region operably linked to the transgene.

E36. The method of E35, wherein in some embodiments the genetic control region comprises a promoter and optionally an enhancer.

E37. The method of any one of E35 to E36, wherein in some embodiments the genetic control region is constitutively active in the cells, or is inducible in the presence of an exogenous environmental factor.

E38. The method of any one of E29 to E32, wherein in some embodiments at least one of the types of pDNA comprises a sequence to express one or more viral helper factors required for parvovirus replication.

E39. The method of E38, wherein in some embodiments the parvovirus is adeno-associated virus (AAV).

E40. The method of E38, wherein in some embodiments the viral helper factors are adenovirus or herpes simplex virus helper factors.

E41. The method of any one of E29 to E32, wherein in some embodiments at least one of the types of plasmid DNA comprises a parvovirus rep gene.

E42. The method of any one of E29 to E32, wherein in some embodiments at least one of the types of plasmid DNA comprises a parvovirus cap gene.

E43. The method of any one of E33 to E42, wherein in some embodiments a first type of plasmid comprises the transgene sequence, and at least a second type of plasmid comprises the sequence for expressing the viral helper factors, the rep gene, or the cap gene.

E44. The method of any one of E33 to E42, wherein in some embodiments a first type of plasmid comprises the transgene sequence and the sequence for expressing the viral helper factors, and at least a second type of plasmid comprises the rep gene or the cap gene.

E45. The method of any one of E33 to E42, wherein in some embodiments a first type of plasmid comprises the transgene sequence and the rep gene, and at least a second type of plasmid comprises the sequence for expressing the viral helper factors or the cap gene.

E46. The method of any one of E33 to E42, wherein in some embodiments a first type of plasmid comprises the transgene sequence and the cap gene, and at least a second type of plasmid comprises the sequence for expressing the viral helper factors or the rep gene.

E47. The method of any one of E33 to E42, wherein in some embodiments a first type of plasmid comprises the transgene sequence, and a second type of plasmid comprises the sequence for expressing the viral helper factors, the rep gene, and the cap gene.

E48. The method of any one of E33 to E42, wherein in some embodiments a first type of plasmid comprises the transgene sequence operably linked to a genetic control region, a second type of plasmid comprises a parvovirus rep gene and a parvovirus cap gene, and a third type of plasmid comprises a sequence for expressing viral helper factors.

E49. The method of any one of E1 to E48, wherein in some embodiments the cells are mammalian cells or insect cells.

E50. The method of E49, wherein in some embodiments the mammalian cells are HEK293 cells, or variants thereof, such as HEK293E, HEK293F, HEK293H, HEK293T, or HEK293FT cells, A549 cells, BHK cells, CHO cells, HeLa cells, or Vero cells.

E51. The method of E49, wherein in some embodiments the insect cells are Sf9 cells, or Sf1 cells.

E52. The method of any one of E1 to E51, wherein in some embodiments the density of viable cells (vc) in the sample at the time of transfection is at least or about 10×10⁶ vc/mL, 15×10⁶ vc/mL, 20×10⁶ vc/mL, 25×10⁶ vc/mL, 30×10⁶ vc/mL, 40×10⁶ vc/mL, or 50×10⁶ vc/mL, or more, or a value between or range comprising any of the foregoing specifically enumerated values, such as about 10×10⁶ to 30×10⁶ vc/mL, 15×10⁶ to 25×10⁶ vc/mL, or 16×10⁶ to 24×10⁶ vc/mL.

E53. The method of any one of E1 to E52, wherein in some embodiments the volume of the cell sample is at least or about 100, 200, 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 6000, 7000, 8000, 9000, or 10000 liters (L), or more, or a value between or range comprising any of the foregoing specifically enumerated values.

E54. The method of any one of E1 to E53, wherein in some embodiments the total volume or mass of transfection cocktail to be added to the cell sample is at least or about 5, 10, 15, 20, 25, 35, or 40 percent, or more, of the volume or mass of the cell sample, or a value between or range comprising any of the foregoing specifically enumerated values; or is at least or about 10, 100, 150, 200, 250, 300, 350, 400, 500, 1000, 1500, or 2000 liters or kilograms, or more, or a value between or range comprising any of the foregoing specifically enumerated values.

E55. The method of any one of E2 to E54, wherein in some embodiments the nucleic acid solution comprises a physiologically compatible fluid, such as water, cell growth media (of the same type or different type as that in which the cells in culture are suspended), dextrose, saline (such as phosphate buffered saline), or other fluids.

E56. The method of any one of E2 to E55, wherein in some embodiments the transfection agent solution comprises a physiologically compatible fluid, such as water, cell growth media (of the same type or different type as that in which the cells in culture are suspended), dextrose, or saline (such as phosphate buffered saline), or other fluids.

E57. The method of any one of E55 to E56, wherein in some embodiments the physiologically compatible fluids are the same.

E58. The method of any one of E55 to E56, wherein in some embodiments the physiologically compatible fluids are different.

E59. The method of any one of E2 to E58, wherein in some embodiments the nucleic acid solution comprises plasmid DNA.

E60. The method of any one of E1 to E59, wherein in some embodiments the volume of transfection cocktail added to the sample of cells is at least or about 0.05, 0.1, 0.15, 0.2, 0.25, 0.3, 0.4, 0.45, or 0.5, or more, as a fraction of the combined volume of the cell sample and transfection cocktail, or a fraction between or range comprising any of the foregoing specifically enumerated values.

E61. The method of any one of E55 to E58, wherein in some embodiments the nucleic acid and transfection reagent solutions are mixed in a ratio ranging from about 5:1 to about 1:5 on a volume or mass basis.

E62. The method of E61, wherein in some embodiments the nucleic acid and transfection reagent solutions are mixed in a ratio of about 1:1 on a volume or mass basis.

E63. The method of E31, wherein in some embodiments the molar ratio of said first and at least second types of plasmids in the transfection cocktail is 1:1, with a deviation not exceeding±20%.

E64. The method of E32, wherein in some embodiments the molar ratio of said first, second and third types of plasmids is 1:1:1, with a deviation not exceeding±20%.

E65. The method of E31, wherein in some embodiments the molar ratio of said first and at least second types of plasmids in the transfection cocktail is other than 1:1.

E66. The method of E32, wherein in some embodiments the molar ratio of said first, second and third types of plasmids is other than 1:1:1.

E67. The method of any one of E26 to E66, wherein in some embodiments transfection cocktail comprises sufficient pDNA such that the cells are transfected with at least or about 0.25, 0.5, 1, 1.5, 2, 3, 4, or 5 micrograms, or more, per million viable cells in the sample (μg/1×10⁶ vc), or a value between or range comprising any of the foregoing specifically enumerated values.

E68. The method of any one of E26 to E66, wherein in some embodiments transfection cocktail comprises sufficient pDNA such that the cells are transfected with at least or about 1, 2.5, 5, 7.5, 12.5, 15, 17.5, 20, 22.5, 25, 27.5, or 30 micrograms, or more, per milliliter of the cell sample, or a value between or range comprising any of the foregoing specifically enumerated values.

E69. The method of any one of E14 to E24, wherein in some embodiments transfection cocktail comprises sufficient PEI such that the cells are transfected with at least or about 0.5, 1, 2.5, 5, 10, or 15 micrograms, or more, per million viable cells in the sample (μg/1×10⁶ vc), or a value between or range comprising any of the foregoing specifically enumerated values.

E70. The method of any one of E26 to E69, wherein in some embodiments the ratio of mass of PEI to mass of pDNA in the transfection cocktail ranges from about 10:1 to about 1:10, for example, about 9:1, 8:1, 7:1, 6:1, 5:1, 4:1, 3:1, 2.9:1, 2.8:1, 2.7:1, 2.6:1, 2.5:1, 2.4:1, 2.3:1, 2.2:1, 2.1:1, 2:1, 1:1, 1:2, 1:3, 1:4, 1:5, 1:6, 1:7, 1:8, 1:9, or some other ratio between or range of ratios comprising any of the foregoing specifically enumerated ratios.

E71. The method of any one of E1 to E70, wherein in some embodiments the method further comprises mixing the transfection cocktail and cell sample which, in some embodiments, can be performed in a stirred tank bioreactor with a power input per volume of at least or about 20, 30, 40, 50, 60, or 70 watts per cubic meter (W/m 3), or more, or a value between or range comprising any of the foregoing specifically enumerated values.

E72. The method of any one of E1 to E71, wherein in some embodiments the method further comprises incubating the transfected cells for time and under conditions sufficient for production of a biological product encoded by the transfected nucleic acid.

E73. The method of any one of E1 to E71, wherein in some embodiments the method further comprises incubating the transfected cells for time and under conditions sufficient for production of a recombinant AAV vector.

E74. The method of any one of E72 to E73, wherein in some embodiments incubation is performed for at least or about 12, 24, 36, 48, 56, 72, 84, or 96 hours, or more, or a value between or range comprising any of the foregoing specifically enumerated values.

E75. The method of any one of E1 to E74, wherein in some embodiments the method further comprises concentrating the transfected cells and removing at least a portion of the culture media.

E76. The method of any one of E73 to E74, wherein in some embodiments the method further comprises lysing the transfected cells.

E77. The method of E76, wherein in some embodiments the method further comprises purifying the recombinant AAV vector.

E78. The method of any one of E2 to E77, wherein in some embodiments the nucleic acid and transfection reagent solutions are stored in separate containers before being mixed together.

E79. The method of any one of E2 to E78, wherein in some embodiments the nucleic acid and transfection reagent solutions are mixed in an open or closed chamber in fluid communication with the storage containers.

E80. The method of E79, wherein in some embodiments the mixing chamber is in fluid communication with a container in which the sample of cultured cells is transfected.

E81. The method of any one of E79 to E80, wherein in some embodiments the method further comprises pumping the nucleic acid and transfection reagent solutions from the storage containers into the mixing chamber and thereafter into the cell culture container.

E82. The method of any one of E79 to E81, wherein in some embodiments mixing of the nucleic acid and transfection reagent solutions is effected mechanically, such as by stirring, vortexing, shaking, agitating, or acoustic mixing, or non-mechanically, such as by diffusion or through the mixing effect of fluid flow, whether laminar or turbulent.

E83. The method of any one of E78 to E82, wherein in some embodiments mixing of the nucleic acid and transfection reagent solutions begins at the first locus of fluid communication between the storage containers which, in some embodiments, is a mixing chamber that joins, via at least two inlets, fluid paths leading separately from each storage container to, via at least one outlet, a fluid path leading to the cell culture container.

E84. The method of E83, wherein in some embodiments the fluid path leading from the mixing chamber to the cell culture container divides and then rejoins before reaching said container.

E85. The method of any one of E83 to E84, wherein in some embodiments the fluid path leading from the mixing chamber to the cell culture container is divided by one or more branches that rejoin downstream via intermediate fluid paths to permit uninterrupted fluid flow to the cell culture container.

E86. The method of any one of E83 to E85, wherein in some embodiments the fluid path leading from the mixing chamber to the cell culture container is divided by one or more branches, each having an inlet upstream and two or more ramifying outlets that rejoin downstream via intermediate fluid paths to permit uninterrupted fluid flow to the cell culture container.

E87. The method of any one of E85 to E86, wherein in some embodiments the branches are integral to hollow connectors.

E88. The method of E79, wherein in some embodiments the mixing chamber comprises two inlets in fluid communication with the storage containers, and an outlet in fluid communication with the cell culture container, wherein in some embodiments the angle between each inlet and the outlet is less than, equal to or more than 90 degrees, and whereas in some other embodiments the angle between each respective inlet and the outlet is the same or different.

E89. The method of any one of E83 to E87, wherein in some embodiments the fluid path leading from the mixing chamber to the cell culture container is configured, for at least a portion of its total length, as one or more coils, each of which in some embodiments can be a flat coil, wound helically as around a cylinder or cone (in a single layer or orthocyclically), or wound toroidally.

E90. The method of any one of E79 to E89, wherein in some embodiments the storage containers fluidly communicates with the cell culture container via a plurality of fluid paths, each of which comprises a mixing chamber.

E91. The method of any one of E79 to E90, wherein in some embodiments the mixing chamber comprises or consists of a hollow connector.

E92. The method of any one of E79 to E91, wherein in some embodiments fluid communication occurs via tubes, and/or the fluid paths comprise or consist of tubes.

E93. The method of any one of E79 to E92, wherein in some embodiments Reynold's number Re associated with fluid flow during performance of the method does not exceed a value of 3500 or 4000.

E94. The method of any one of E79 to E92, wherein in some embodiments fluid flow during performance of the method is non-turbulent.

E95. The method of E77, wherein in some embodiments the method is effective to produce a recombinant AAV vector having a titer of at least or about 1×10¹⁰, 5×10¹⁰, 1×10¹¹, 5×10¹¹, 1×10¹², 5×10¹², or 1×10¹³ vector genomes per milliliter (vg/mL) of cell suspension after transfection, or more, or a titer between, or range comprising, any of the foregoing specifically enumerated values.

E96. The method of E95, wherein in some embodiments the recombinant AAV vector titer is determined by ITR qPCR.

E97. The method of E95, wherein in some embodiments the recombinant AAV vector titer is determined by transgene qPCR.

E98. The method of E77, wherein in some embodiments the method is effective to produce a recombinant AAV vector having, after purification by size exclusion chromatography, a UV260/UV280 absorbance ratio of at least or about 0.4, 0.5, 0.6, 0.7, 0.8, 0.9, 1.0, 1.1, 1.2, 1.3, 1.4, 1.5, 1.6, 1.7, or 1.8, or more, or a UV260/UV280 absorbance ratio between, or range comprising any of the foregoing specifically enumerated values.

E99. In another embodiment, the disclosure provides a system for continuously transfecting a sample of cells in culture with nucleic acid, the system comprising: (i) means for containing a nucleic acid solution, (ii) means for containing a transfection reagent solution, (iii) means for containing the sample of cells in culture, (iv) means for mixing said solutions continuously to form a transfection cocktail, and (v) means for fluid communication from the respective solution containment means to the mixing means and therefrom to the cell sample containment means.

E100. The system of E99, wherein in some embodiments said system further comprises means for causing fluid communication from the solution containment means of the nucleic acid and transfection reagent solutions to the mixing means and therefrom to the cell sample containment means.

E101. The system of any one of E99 to E100, wherein the system comprises: (i) a container for a nucleic acid solution, (ii) a container for a transfection agent solution, (iii) a mixing chamber in fluid communication with each of said containers, (iv) a container for the cell sample in fluid communication with said mixing chamber, and (v) at least one pump.

E102. The system of any one of E99 to E101, wherein the system is configured to continuously form and deliver at least 50 L of a transfection cocktail to at least 500 L of cells in suspension culture in 60 minutes or less, wherein the transfection cocktail is formed by mixing solutions separately comprising a nucleic acid and a transfection reagent, and wherein the transfection cocktail, once formed, is delivered to the cells in 30 minutes or less.

E103. The system of E102, wherein the system is configured to continuously form and deliver said at least 50 L of transfection cocktail to the cells in suspension culture in 45 minutes or less, and wherein the transfection cocktail, once formed, is delivered to the cells in 15 minutes or less.

E104. The system of any one of E102 to E103, wherein the system is configured to continuously form and deliver said at least 50 L of transfection cocktail to the cells in suspension culture in 30 minutes or less, and wherein the transfection cocktail, once formed, is delivered to the cells in minutes or less.

E105. The system of any one of E99 to E101, wherein the system is configured to continuously form and deliver at least 100 L of a transfection cocktail to at least 1000 L of cells in suspension culture in 60 minutes or less, wherein the transfection cocktail is formed by mixing solutions separately comprising a nucleic acid and a transfection reagent, and wherein the transfection cocktail, once formed, is delivered to the cells in 30 minutes or less.

E106. The system of E105, wherein the system is configured to continuously form and deliver said at least 100 L of transfection cocktail to the cells in suspension culture in 45 minutes or less, and wherein the transfection cocktail, once formed, is delivered to the cells in 15 minutes or less.

E107. The system of any one of E105 to E106, wherein the system is configured to continuously form and deliver said at least 100 L of transfection cocktail to the cells in suspension culture in minutes or less, and wherein the transfection cocktail, once formed, is delivered to the cells in 10 minutes or less.

E108. The system of any one of E99 to E107, wherein the system is configured so that Reynold's number Re associated with fluid flow does not exceed a value of 3500 or 4000.

E109. In another embodiment, the disclosure provides a biological product made by the method of any of the embodiments of E1 to E98.

E110. The product of E109, wherein in some embodiments said product is a protein, a nucleic acid, a vaccine or component thereof, a virus, or a recombinant viral vector.

E111. The product of E110, wherein in some embodiments the biological product is a protein selected from the group consisting of: an antibody, a protein fusion with an immunoglobulin Fc domain, a clotting factor, an enzyme, and a zymogen.

E112. The product of E110, wherein in some embodiments the biological product is a recombinant viral vector selected from the group consisting of: adenoviral vector, adeno-associated viral (AAV) vector, lentiviral vector, and retroviral vector.

### BRIEF DESCRIPTION OF THE DRAWINGS

FIG. 1. Exemplary system for transfection illustrating means for separately containing (in this embodiment, 50 L bioprocess container) transfection reagent (in this embodiment, PEI) and nucleic acid (in this embodiment, three DNA plasmids for producing recombinant AAV vectors), pump means (in this embodiment peristaltic pumps), mixing means (in this embodiment, a T-connector serving as a static in-line mixer), cell containment means (in this embodiment, a 250 L capacity single use stirred tank bioreactor), as well as fluid communication means (in this embodiment, thermoplastic elastomer tubing) from the solution containers to the T mixer and therefrom to the bioreactor. As illustrated in this embodiment, the tubing from the mixer to the bioreactor is coiled to improve mixing of transfection cocktail.

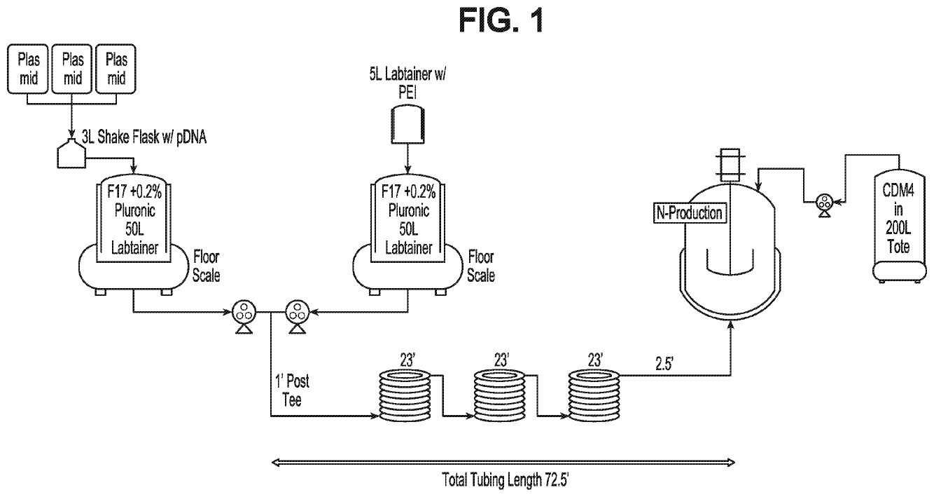

FIG. 2. Exemplary system for transfection illustrating use of two parallel subassemblies for delivering transfection cocktail to cells. Each subassembly is connected by tubes to separate containers for transfection reagent (in this embodiment, PEI) and nucleic acid (in this embodiment, plasmid DNA) in solution, and comprises a peristaltic pump to draw PEI or pDNA solution from its respective container, a T connector serving as a static in-line mixer of the PEI and pDNA solutions, a coiled tube for further mixing and incubation of transfection cocktail as it is pumped from the T mixer to the bioreactor, and finally a bioreactor (in this embodiment, a 2000 L bioreactor) containing the cells to be transfected. Utilization of two or more such subassemblies in parallel permits even large volumes of transfection cocktail to be delivered to cells in relatively short periods of time.

FIG. 3. Graph of results from experiments designed to test titer of a recombinant AAV vector produced from HEK293 cells transfected using bolus method as a function of the time transfection cocktail was incubated before being added to the cells. In these experiments, incubation times between 2 and 125 minutes were tested. Cells were grown and transfected in mL scale culture.

FIG. 4. Graph of results from experiments designed to test titer of a recombinant AAV vector produced from HEK293 cells transfected using bolus method as a function of the time transfection cocktail was incubated before being added to the cells. In these experiments, incubation times between 1.5 and 20 minutes were tested. Cells were grown and transfected in mL scale culture.

FIG. 5. Graph of results from experiments designed to test titer of a recombinant AAV vector produced from HEK293 cells transfected continuously using a static in-line mixer as a function of the time transfection cocktail was incubated before being added to the cells. In these experiments, incubation times between 0.75 and 5 minutes were tested. Cells were grown and transfected in 1 L scale culture.

FIG. 6. Graph of results from experiments designed to test the proportion of recombinant AAV vector produced from continuously transfected HEK293 cells containing full capsids (as reflected by the SEC A260/A280 UV absorbance ratio) as a function of the viable cell density (VCD) at the time of transfection. Incubation time (90 secs) and addition time (30 min) were held constant. Cells were grown and transfected in 1 L scale culture.

FIG. 7. Graph of results from experiments designed to test the titer of recombinant AAV vector produced from continuously transfected HEK293 cells as a function of the viable cell density (VCD) at the time of transfection. Incubation time (90 secs) and addition time (30 min) were held constant. Cells were grown and transfected in 1 L scale culture.

FIG. 8. Graph of results from experiments designed to test the proportion of recombinant AAV vector produced from continuously transfected HEK293 cells containing full capsids (as reflected by the SEC A260/A280 UV absorbance ratio) as a function of the amount of plasmid DNA used in the transfection (as μg per million cells). Incubation time (90 secs) and addition time (30 min) were held constant. Cells were grown and transfected in 1 L scale culture.

FIG. 9. Graph of results from experiments in which the relative potency of AAV vectors produced using continuous flow transfection systems at different scales and under different flow conditions is compared to the Reynolds number (Re) calculated for each experiment. Re values above about 3500 were associated with lower relative vector potency and lower percentage of full capsids. In the figure, circles refer to data from vector produced at 10 L scale, squares refer to data from vector produced at 250 L scale, and triangles refer to data from vector produced at 2000 L scale.

### DETAILED DESCRIPTION OF THE INVENTION

### Methods for Transfecting Host Cells

As used herein, transfection (and related terms like transfect) refers to processes that introduce nucleic acids into eukaryotic cells by non-viral methods, including chemical methods or physical methods. Thus, a transfected cell is one that has had exogenous nucleic acid introduced into it through a process of transfection. As known in the art, transfection can be transient or stable. With transient transfection, the transfected DNA or RNA exists in the cells or their progeny for a limited period of time and, in the case of DNA, does not integrate into the genome. With stable transfection, DNA introduced into the cell can persist for long periods either as an episomal plasmid, or integrated into a chromosome. Usually, to produce stably transfected cells, a plasmid containing a selection marker, as well as the gene or genes for expressing the desired biological product, is transfected into the cells which are then grown and maintained under selective pressure, i.e., conditions that kill non-transfected cells or transfected cells from which the exogenous DNA, including its selection marker, are lost for some reason. For example, plasmids can contain an antibiotic resistance gene and transfected cells can be selected for by adding the antibiotic to the media in which the cells are grown. In some embodiments, the gene for producing the biological product introduced into stably transfected host cells is under the control of an inducible promoter and is not expressed, or only at a low level, unless an environmental factor, such as a drug, metal ion, or temperature increase, which induces the promoter, is introduced as the cells are grown. The methods and systems of the disclosure can be used to prepare both stably and transiently transfected cells.

In some embodiments transfection is chemically-mediated, wherein a transfection reagent forms complexes with nucleic acid that are more readily taken up by a recipient host cell than uncomplexed nucleic acid. Thus, transfection reagent refers to a chemical compound or composition comprising chemical compounds added to nucleic acid for enhancing the uptake of the nucleic acid into a host cell. A mixture or combination of transfection reagent and nucleic acid is known as a transfection cocktail.

As described further in the Examples, the inventors observed a dependency between the time of transfection cocktail incubation (that is, after mixing together the transfection reagent and nucleic acid) and transfection efficiency. More specifically, the longer the period after preparing the transfection cocktail until the cocktail was added to cells to transfect them, the lower the apparent transfection efficiency. While not wishing to be bound by any particular theory of operation, this effect could be due to increasing size with time of the particulate complexes that form between transfection reagent and the nucleic acids in solution, such that there is some optimum size (which may not be precisely known) above which transfection efficiency begins to decline. Although this effect was observed in the specific context of PEI as the transfection reagent, plasmid DNA as the nucleic acid, and yield of adeno-associated viral (AAV) vectors produced from the transfected cells, the inverse relationship between incubation time and transfection efficiency is not considered to be unique to this combination of variables, but is instead characteristic of many chemically-based transfection systems, types of nucleic acid, and products produced by transfected cells.

As noted above, delivering transfection cocktail to cells can be accomplished relatively rapidly when the volumes are modest (for example a few liters or so, which is suitable for laboratory use) such that the delay between preparing the transfection cocktail (by mixing together all needed components) and delivering it to cells is short (minutes to tens of minutes) and therefore does not significantly impact transfection efficiency. As will be appreciated, however, as the volume of cells grown in culture scales upward, it becomes increasingly technically challenging to prepare commensurately large volumes of transfection cocktail and then deliver it to the cells without a delay that reduces transfection efficiency. This can be so for various reasons, but particularly relevant is time to effect thorough mixing of the cocktail and time to deliver the cocktail to cells, so as to ensure thorough distribution throughout the cell culture while maintaining adequate cell viability.

As for mixing, it takes longer to combine and thoroughly mix larger volumes of transfection reagent and nucleic acid, which is needed to ensure that as much nucleic acid as possible is complexed. And, this delay cannot necessarily be reduced much by faster mixing, as the mixing rate cannot be raised too high before generating shear forces that can interfere with complex formation, or damage the nucleic acid. Viscosity differences between the solutions containing transfection reagent and nucleic acid can yet demand more time before thorough mixing is achieved. The delay associated with the mixing process can also cause particles to form at different times as mixing progresses. Particles formed earlier can increase in size beyond an optimum as younger particles just start to form. Thus, the transfection cocktail can contain a range of particle sizes, only a minority of which could be optimal for transfection. A second cause of delay which can result in excessive incubation time is associated with the time needed to deliver the cocktail to the cells. There are at least two issues. As is well appreciated, certain transfection reagents, such as PEI, can be toxic to cells and should be added to the cell culture slowly enough, even with mixing, to avoid areas of excessively high local concentration to maintain sufficient cell viability. Another factor is that cocktail should be added slowly enough to be thoroughly distributed and mixed throughout the cell culture in order to achieve transfection of most of the cells. Both factors necessitate some period of delay before the entire volume of transfection cocktail is ultimately delivered to the cells; the cocktail cannot just be added all at once.

Conventionally, solutions comprising transfection reagent (or comprising components that when combined generate transfection reagent) and separately nucleic acids are combined in a beaker, mixing tank or some other suitable container, and then mixed together, such as with a stir bar, or in larger vessels with a mixing propeller, paddles, or the like. Then, once the cocktail is thoroughly mixed, it might be incubated for some period of time sufficient to permit particles of complexed transfection reagent and nucleic acid to form, after which the cocktail would be added to cells in culture in a flask or bioreactor. The adding step can be done in a variety of ways known in the art, for example, by pumping the cocktail into the cell culture, suspending a container holding the cocktail above the cell culture vessel and allowing gravity to feed the cocktail through a tube into the culture media, or by pressurizing a closed container holding the cocktail so as to force the cocktail through a tube or pipe connected to the culture vessel and into the media. For the reasons summarized above, however, these approaches are poorly suited when the cocktail volume is large. The delays required for thorough mixing and transport of tens to hundreds of liters of cocktail into the cell culture tank increase with volume, eventually reducing transfection efficiency and/or productivity of a desired biological product synthesized by the transfected cells to an unacceptable degree.

Seeking to maintain a high level of transfection efficiency at the large volumes of transfection cocktail and cultured cells associated with industrial scale bioprocesses, the inventors have developed improved methods and associated systems for preparing and delivering transfection cocktail to cells. In particular, though non-limiting embodiments, these methods include preparing and delivering large volumes of transfection cocktail continuously (and in some embodiments simultaneously), thereby ensuring thorough mixing of transfection reagent and nucleic acid, and thereafter delivery to cells, so as to effect transfection without the undue delay characteristic of conventional methods. In this way, high levels of transfection efficiency can be achieved, even for purposes of making complicated multi-component biological products, such as gene therapy vectors, at industrial scale.

According to some embodiments, methods of transfecting host cells comprise the step of preparing a transfection cocktail and contacting a sample of host cells with transfection cocktail, such as by adding or delivering transfection cocktail to such sample. Such methods can be carried out using systems for transfection as described herein. As used herein, “transfection cocktail” is a mixture of a transfection reagent and nucleic acid in liquid suspension or solution of such types, and in such amounts and proportions, as are suitable for transfecting host cells. In some embodiments, solutions comprising transfection reagent and nucleic acids may first be prepared separately and subsequently mixed together to prepare or form transfection cocktail. In this fashion, the incubation time of the transfection cocktail can be carefully controlled in view of its potential impact on transfection efficiency, as explored further in the Examples. Methods and systems of the disclosure can be employed to transfect a variety of cell types using different transfection reagents and types of nucleic acids to efficiently produce different biological products.

After preparing solutions separately comprising transfection reagent and nucleic acid for use in a transfection, the solutions are mixed together to prepare or form transfection cocktail to be delivered to a sample of cells for transfection. In some embodiments mixing is effected using mixing means of systems for transfection described herein. In some embodiments, the step of preparing transfection cocktail is carried out in a discrete step temporally separate from the step of contacting cells with transfection cocktail. Preparing all transfection cocktail in one discrete step followed by contacting cells is known as bolus transfection, whereas preparing all transfection cocktail in a plurality of discrete steps each followed by contacting cells is known as segmented bolus transfection. In other embodiments of the methods, the process is carried out continuously, meaning that the preparing or forming of transfection cocktail occurs simultaneously with (for at least some period) the contacting of cells with transfection cocktail. In some embodiments of the continuous process, a portion of transfection cocktail is just starting to be prepared or formed at the same time that another portion, formed earlier, is being contacted to cells for transfection, such as by addition or delivery of that portion to a sample of cells. Continuous processes, however, in some embodiments, can be interrupted, such that the total volume of transfection cocktail is added to a cell sample discontinuously. With reference to systems of the disclosure, such interruption can be carried out by inactivating pump means for one or more periods and then optionally restarting such pump means until the total volume of transfection cocktail has been formed and delivered to the cell sample.

The transfection methods of the disclosure can be described using two time factors. The first time factor, incubation time, is the total time that transfection cocktail, or portion thereof, is incubated before being added to a sample of cells for transfection. Incubation time commences when transfection reagent solution and nucleic acid solutions are first brought into contact and start to mix together to form transfection cocktail and ends when the transfection cocktail so formed is added or delivered to the cell sample. In reference to systems of the disclosure, incubation time begins when transfection reagent solution and nucleic acid solution first contact each other in or at mixing means and ends when the transfection cocktail so formed exits fluid communication means into cell containment means. The second time factor, addition time, is the time required for a predetermined volume, such as the total volume, of transfection cocktail to be added or delivered (including in a continuous process) to a sample of cells for transfection. In reference to systems of the disclosure, addition time begins when transfection reagent and nucleic acid solutions are caused to start flowing to mixing means and ends when the last portion of transfection cocktail to be added or delivered has been added or delivered to the sample of cells in cell containment means. Systems of the disclosure can be configured to control incubation time, to ensure that it is of sufficiently short duration that transfection is highly efficient, as well as to control total addition time.

### Transfection Reagent

The methods of the disclosure can be used with any suitable chemically-based transfection reagent. In some embodiments, transfection reagent solution is prepared by dissolving transfection reagent in powder or other solid form in a suitable solvent, or diluting a concentrated stock solution of transfection reagent with a suitable diluent. Any biocompatible solvents or diluents known in the art to support complexation of the chosen transfection reagent and nucleic acids can be used, non-limiting examples of which include saline, phosphate-buffered saline, dextrose solution, Ringer's lactate solution, cell growth media, or water. Such solvents and diluents can be supplemented with other ingredients as known in the art, such as salts, buffers, or detergents. In other embodiments, transfection reagent requires the combination of two or more chemical components, any of which may be in solid or liquid form. Transfection reagent solutions can be homogenous, containing one type of transfection reagent, or can be heterogenous, containing different types, or one main type that is itself heterogenous by being provided in a range of molecular weights, as having different stereochemical forms, or some other type of heterogeneity. Once prepared, transfection reagent solution can be stored temporarily in suitable containment means of systems, as described herein.

In some embodiments, the transfection reagent can be a cationic compound having the capacity to condense nucleic acid (e.g., DNA) including, without limitation, cationic monomers and polymers, and can include cationic polysaccharides, polypeptides, other polymers, and lipids, including cationic liposomes and lipid nanoparticles. Cationic compounds for use in the methods and systems of the disclosure may be linear, branched, or of other configurations, and may be derivatized to modify their properties in desirable ways. Cationic compounds for use as transfection reagents can be provided in any suitable molecular weight which, in non-limiting embodiments, can range from about 50 to about 1,250,000 daltons (Da), with other molecular weights and ranges possible.

Cationic compounds can include, without limitation, chitosan; protamine; poly-L-lysine (PLL); polyamines (PA); polyalkylenimine (PAI); polyethylenimine (PEI), or derivatives thereof; poly[a-(-aminobutyl)-L-glycolic acid]; polyamidoamine; poly(2-dimethylamino) ethyl methacrylate (PDMAEMA); polyhistidine; histones; polyarginine; poly(4-vinylpyridine); poly(vinylamine); poly(4-vinyl-N-alkyl pyridinium halide); N4′-(2,3-dioleyloxy)propyl]-N,N,N-trimethylammonium chloride (DOTMA); N-[1-(2,3-dioleoyloxy)propyl]-N,N,N-trimethylammonium methyl-sulfate (DOTAP); 1,2-dioleyloxy-3-dimethylaminopropane (DODMA); N1-[2-((1S)-1-[(3-aminopropyl)amino]-4-[di(3-amino-propyl)amino]butylcarboxamido)ethyl]-3,4-di[oleyloxy]-benzamide (MVL5); O-alkyl phosphatidylcholines; dimethyldioctadecylammonium bromide (DDAB); 3β-[N—(N′,N′-dimethylaminoethane)-carbamoyl]cholesterol hydrochloride (DC-Cholesterol·HCl); N-(4-carboxybenzyl)-N,N-dimethyl-2,3-bis(oleoyloxy)propan-1-aminium (DOBAQ); 1,2-dimyristoyl-3-dimethylammonium-propane (DAP); N⁴-cholesteryl-spermine HCl salt (GL67).

Exemplary commercially available transfection reagents include, without limitation, PH MAX (Polysciences), MAXGENE (Polysciences), FUGENE (Roche), TRANSFECTIN (Bio-Rad), CLONFECTIN (Clontech), DREAMFECT (OZ Biosciences), TRANSFAST (Promega), ESCORT (Sigma-Aldrich), LIPOGEN (InvivoGen), TRANSIT-EXPRESS (Mirus), GENEJUICE (Novagen), SUPERFECT (Qiagen), GENEJAMMER (Stratagene), LIPOFECTAMINE2000 (Invitrogen), X-TREMEGENE (Roche), SIIMPORTER (Upstate), BLOCK-IT (Invitrogen), RNAIFECT (Qiagen), GENEERASER (Stratagene), RIBOJUICE (Novagen), HIPERFECT™ (Qiagen), GENESILENCER (Genlantis), SIPORT (Ambion), SILENTFEC (Bio-Rad), SIFECTOR (B-Bridge), TRANSIT-SIQUEST (Mirus), TRANSIT-TKO (Minis), JETSI (Polyplus), PEI-PRO (Polyplus), FECTOVIR (Polyplus), and CODEBREAKER (Promega).

### PEI

In some embodiments, the polycationic transfection reagent is polyethylenimine (PEI). PEI is available in many forms and molecular weights, and any form or molecular weight of PEI known in the art to be effective for transfection of host cells can be used in the methods and systems of the disclosure. In some embodiments, PEI can be linear, branched, or be in the form of a comb, network, or dendrimer, or some other form. In some embodiments, PEI can be in a salt form (e.g., HCl salt) or in a non-ionized form as a free base. Preparations of PEI can be homogenous, meaning they contain PEI of a single form and/or size, or heterogenous, meaning they contain PEI of multiple forms and/or size. In some embodiments, PEI can be functionalized, derivatized, or modified by chemically attaching to one or more atoms in PEI various other polymers, ligands, substituents, or moieties, non-limiting examples of which include carbohydrates, lipids, polypeptides, chitosan, mannosylated chitosan, galactosylated chitosan, dextran, pullulan, polyethylene glycol, alkyl chains, cholesterol, poly(ethylene oxide)-b-poly(propylene oxide)-b-poly(ethyleneoxide) block copolymers, folic acid, transferrin, amino acids, peptides, or lysine-histidine peptides, with many others being possible. In some embodiments, the chemical substitution occurs at one or more primary, secondary or tertiary amines in PEI polymer chains. Compositions or preparations of PEI can comprise mixtures and combinations of one or more types of functionalized, derivatized, or modified forms of PEI.

For use in the methods and systems of the disclosure, solid PEI, or concentrated solutions of PEI, can be dissolved or diluted in suitable solvents or diluents to prepare stock solutions of PEI. Exemplary non-limiting solvents that may be used to dissolve or dilute PEI include polar solvents, such as water, ethanol, or acetone, or mixtures of these solvents, or other polar solvents known in the art, with the optional addition of other ingredients, such as salts (e.g., NaCl), or buffers. The pH of stock solutions of PEI may be adjusted to any desired value or range of pH, such as about pH 4 to 9, pH 5 to 8, pH 7 to 8, or some other range of pH.

In some embodiments, preparations of PEI are heterogenous by comprising PEI molecules with different numbers of subunits. As known in the art, the molecular weight (MW) of PEI (such as linear or branched PEI) in such preparations can be expressed in different ways. For example, in some embodiments, the MW can be the number average MW, which may be abbreviated Mn. Thus, in some embodiments, the number average MW (Mn) of PEI for use in the methods and systems of the disclosure can be at least or about 0.1, 0.2, 0.3, 0.4, 0.5, 0.6, 0.7, 0.9, 1, 1.1, 1.2, 1.3, 1.4, 1.5, 1.6, 1.7, 1.8, 1.9, 2, 2.5, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, 800 kDa, or more, or a Mn between, or range comprising, any of the foregoing values. In other embodiments, the MW of PEI in a heterogenous preparation of PEI can be expressed as the weight average MW, which may be abbreviated Mw. Thus, in some embodiments, the weight average MW (Mw) of PEI (such as linear or branched PEI) for use in the methods and systems of the disclosure can be at least or about 0.1, 0.2, 0.3, 0.4, 0.5, 1, 1.5, 1.6, 1.7, 1.8, 1.9, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, 800 kDa, or more, or a Mw between, or range comprising, any of the foregoing values. Molecular weight of PEI in preparations of PEI can be determined using different analytic methods known the art, such as gel permeation chromatography, size exclusion chromatography, laser light scattering, matrix-assisted laser desorption/ionization mass spectroscopy, or other methods.

If the number average and weight average molecular weights of a PEI preparation are known then the polydispersity index (PDI) of the preparation can be calculated as the ratio Mw/Mn, which quantifies the heterogeneity of the PEI in the preparation. If the PDI has a value exactly 1, then the PEI is monodisperse or homogenous, meaning the PEI polymers in the preparation contain the same number of subunits. However, PDI values greater than 1 indicate increasing heterogeneity as reflected in the width of the molar mass distribution of the polymers. In some embodiments, preparations of PEI used in the methods and systems of the disclosure can have a PDI of exactly 1, or more than 1, such as at least or about 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, 3.0, 3.1, 3.2, 3.3, 3.4, 3.5, 4, 5, 6, 7, 8, 9, 10, 11, 12, 13, 14, 15, 16, 17, 18, 19, 20, 25, 30, or higher, or a PDI value between, or range of PDI values comprising, any of the foregoing values.

As is known in the art, chemical synthesis of linear PEI can result in the incomplete removal of N-propionyl groups, the extent of which can be estimated by NMR spectroscopic analysis. Furthermore, incomplete removal of such N-propionyl groups reduces the number of prontonable nitrogens in the PEI polymer chain, which may reduce the effectiveness by which the PEI can condense with DNA or other nucleic acid for purposes of transfection. If desired, PEI preparations in which the PEI is not fully deacylated can be hydrolyzed, such as by treating the PEI with HCl, to remove all or substantially all remaining N-propionyl groups. Such fully hydrolyzed PEI may be more effective as a transfection reagent compared to only partially hydrolyzed PEI. Nevertheless, even partially hydrolyzed PEI may still be effective as a transfection reagent. Thus, in some embodiments, PEI for use in the methods and systems of the disclosure can be at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% free of N-propionyl groups (i.e., depropionylated) as determined by NMR spectroscopic analysis, or a percentage between, or range of percentages comprising, any of the foregoing percentages.

As is known in the art, linear PEI molecules contain primary amine groups at each end of the polymer chain, and secondary amine groups along the polymer backbone, whereas branched PEI molecules additionally possess tertiary amine groups where branch points occur. The ratio of the average number of primary amine groups to secondary amine groups in linear PEI, and the ratio of the average number of primary to secondary to tertiary amine groups in branched PEI can vary depending on the length and/or complexity of such molecules, and PEI for use in the methods and systems of the disclosure can possess any suitable ratio of primary amine groups to secondary amine groups, or ratio of primary to secondary to tertiary amine groups. Thus, for example, in some non-limiting embodiments, branched PEI can have primary, secondary, and tertiary amine groups in a ratio of approximately 1:2:1, or some other ratio of primary, secondary, and tertiary amine groups.

Examples of commercially available PEI preparations include, without limitation, LUPASOL® G20, LUPASOL® FG, LUPASOL® G35, LUPASOL® P, and LUPASOL® 1595 (all from BASF); EPOMIN® SP-003, EPOMIN® SP-006, EPOMIN® SP-012, EPOMIN® SP-018, EPOMIN® SP-200, EPOMIN® SP-1000, and EPOMIN® SP-1050 (all from Nippon Shokubai); and TRANSPORTER S®, PEI PEI MAX®, and MAXGENE® (all from Polysciences). Additional information about PEI and its uses may be found in, e.g., Thomas, M, et al., Full deacylation of polyethylenimine dramatically boosts its gene delivery efficiency and specificity to mouse lung, PNAS 102(16):5679-84 (2005); Pandey, A P and Sawant, K K, Polyethylenimine: A versatile, multifunctional non-viral vector for nucleic acid delivery, Mat. Sci. Eng. C, 68:904-18 (2016); Godbey, W T, et al., Size matters: Molecular weight affects the efficiency of poly(ethylenimine) as a gene delivery vehicle, J. Biomed. Mats. Res. 45(3):268-75 (1999); Boussif, O, et al., A versatile vector for gene and oligonucleotide transfer into cells in culture and in vivo: Polyethylenimine, PNAS 92:7297-301 (1995); Virgen-Ortiz, J J, et al., Polyethylenimine: a very useful ionic polymer in the design of immobilized enzyme biocatalysts, J. Mater. Chem. B 5:7461-90 (2017); Park, I H and Choi, E-J, Characterization of branched polyethylenimine by laser light scattering and viscometry, Polymer 37(2):313-9 (1996); Kircheis, R, et al., Design and gene delivery activity of modified polyethylenimines, Adv. Drug Deliv. Rev. 53:341-58 (2001); Wong, S Y and Putnam, D, The stochastic effect of polydispersity on polymeric DNA delivery vectors, J. Appl. Polym. Sci. 135:45965 (2018); Baker, A, et al., Polyethylenimine (PEI) is a simple, inexpensive and effective reagent for condensing and linking plasmid DNA to adenovirus for gene delivery, Gene Ther. 4:773-82 (1997); von Harpe, A, et al., Characterization of commercially available and synthesized polyethylenimines for gene delivery, J. Control. Rel. 69:309-22 (2000); Ulasov, A V, et al., Properties of PEI-based Polyplex Nanoparticles That Correlate With Their Transfection Efficacy, Mol. Ther. 19(1):103-12 (2011); Hou, S, et al., Formation and structure of PEI/DNA complexes: quantitative analysis, Soft Matt. 7:6967-72 (2011).

### Nucleic Acids

The methods and systems of the disclosure can be used with any suitable nucleic acid for which it is desired to transfect host cells. In some embodiments, nucleic acid in solution is prepared by dissolving nucleic acid in solid form (for example, as a lyophilisate) in a suitable solvent, or diluting a concentrated nucleic acid stock solution in a suitable diluent. A nucleic acid stock solution can be stored frozen before use, if desired, to enhance its stability. Any biocompatible solvent or diluent known in the art to support complexation of the chosen transfection reagent and nucleic acid can be used, non-limiting examples of which include saline, phosphate-buffered saline, dextrose solution, Ringer's lactate solution, cell growth media, or water. Such solvents and diluents can be supplemented with other ingredients as known in the art, such as buffers, salts, or detergents. The solvent or diluent used to prepare the nucleic acid solution for transfection could be the same or different as the one used to prepare the transfection reagent solution. Once prepared, nucleic acid solution can be stored temporarily in suitable containment means of systems, as described herein.

The terms “nucleic acid” is used herein to refer to all forms of nucleic add, including deoxyribonucleic acid (DNA) and ribonucleic acid (RNA), including oligonucleotides and polynucleotides. DNA can include, without limitation, single stranded DNA (ssDNA), double stranded DNA (dsDNA), triplex DNA, genomic DNA, complementary DNA (cDNA), antisense DNA, plasmid DNA, other episomal forms of DNA, chromosomes (including, for example, bacterial and yeast artificial chromosomes), phage DNA (such as lambda phage), cosmid DNA or bacmid DNA. RNA can include, without limitation, single stranded RNA, double stranded RNA, messenger RNA (mRNA), or pre-mRNA unspliced message), ribosomal RNA (rRNA), transfer RNA (tRNA), short hairpin RNA, micro RNA (miRNA), antisense RNA, small or short interfering RNA (siRNA). Nucleic adds, whether DNA or RNA, include naturally occurring, synthetic, and intentionally modified or altered sequences (e.g., variant nucleic acid). Nucleic acid can have any sequence of nucleobases, which in many embodiments are the adenine (A), cytosine (C), and guanine (G), found in both RNA and DNA, and the thymine (T) of DNA and the uracil (U) of RNA, but nucleic acids can, in other embodiments, include less usual bases, such as hypoxanthine in the nucleoside inosine (I) (or deoxyinosine). Nucleic acids for use in the methods and systems of the disclosure can also include nucleic acids incorporating nucleotides comprising variant or modified bases, nucleoside sugars, or phosphate groups intended to alter the structure and/or function of the nucleic acid, as well as nucleic acids modified or derivatized chemically or enzymatically to achieve similar goals. In some embodiments, nucleic acids for use in the methods and systems of the disclosure can be complexed with protein to form ribonucleo-protein (RNP) complexes, which can be transfected.

In some embodiments the nucleobase sequence comprised by a nucleic acid encodes one or more polypeptides, or codes for one or more functional RNA molecules, whereas in other embodiments a nucleic acid can comprise a nucleotide sequence with inherent catalytic activity (e.g., a ribozyme), or which can be incorporated into a supramolecular structure, such as a virus or recombinant vector derived from a virus, such as adenovirus, adeno-associated virus (AAV), or lentivirus.

In some embodiments, nucleic acid for use in the methods and systems of the disclosure is plasmid DNA (abbreviated pDNA). Classically, plasmids are circular, double-stranded extrachromosomal DNA elements found in bacteria that replicate independently of the bacterial chromosome and carry genes responsible for various non-essential bacterial properties, such as enzymes that confer antibiotic resistance (for example, amp or kan genes). As is well known, plasmids can be modified in various ways using genetic engineering techniques, including by adding new genes and other genetic information. Such recombinant plasmids can be replicated to high copy number in bacteria, purified, and then used to transfect eukaryotic host cells in which the genetic information embodied in the plasmid can direct biosynthesis of biological products. Plasmids can have different conformations, including supercoiled, relaxed circular, nicked open-circular, or linear, with others possible. Nucleic acids, including plasmids, for use in the methods and systems of the disclosure can be any suitable size, for example, about 500 base pairs to 3 million base pairs, or some other size, and can be prepared using any technique familiar to those of ordinary skill in the art. Plasmids, for example, can be grown in large amounts in transformed bacteria, after which the plasmids can be isolated and purified using different techniques known in the art.

According to certain embodiments, plasmids for use in the methods and systems of the disclosure can be modified to include any gene capable of directing the production of a desired biological product in cells (transgene, or gene of interest), such as, without limitation, a polypeptide. Such genes can be from any species, including without limitation species of animal (including, without limitation mammalian species, such as, without limitation, human), plant, fungus, or bacteria. As known in the art, other genetic regulatory sequences can be included in plasmids to direct the host cells' transcriptional, translational, and post-translational machinery to efficiently produce desired biological products. For example, in some non-limiting embodiments, in addition to a gene, plasmids can be engineered to include promoters to guide transcriptional initiation of the gene, and optionally enhancers to augment the rate of transcription. Promoter and/or enhancers can be constitutive, or tissue-specific so that they are only active, or are more active, in certain cell types, or inducible in response to exogenous signals, such as certain drugs, heavy metals, heat shock, or the like. In other embodiments, transcriptional terminators, such as polyadenylation signal sequences, can be included to instruct the host cell to stop transcribing from the gene in the plasmid. In yet other embodiments, non-coding exons or introns can be included (which may or may not interrupt coding sequence), which in some cases have been demonstrated to stabilize transcripts or allow alternative splicing. The gene, in some embodiments, can be provided with a start codon including a Kozak consensus sequence to enhance translational initiation at the start codon. In other embodiments, however, the gene can be provided with a non-consensus start codon, which allows translation of multiple gene products through use of alternative start codons elsewhere in the gene. In some embodiments, the gene can be provided with one or more stop codons. In some embodiments, the gene sequence is naturally occurring, but in other embodiments, the gene sequence can be codon-optimized to match the preferred codon frequency in the species from which the cells are derived, for example, human codon-optimized. The genetic regulatory sequences can be arranged in any order known in the art to be functional. For example, an enhancer could be positioned 5′ of a gene, but could also be positioned 3′ of a gene and still function to enhance transcription in some cases.

Plasmids for use in the methods and systems of the disclosure can originate from any species or strain of bacteria, and can be any size sufficient to comprise all genetic information required to function as desired including, without limitation, an origin of replication, selection marker (such as an antibiotic resistance gene), multiple cloning site, gene of interest, as well as genetic control regions to guide transcription and/or translation. Nucleic acid for transfection can comprise a single type of plasmid, or a plurality of independent types of plasmids (for example, 2, 3, 4 or more), which may be similar or different in size, and each containing some unique genetic information relative to the other types of plasmids in the transfection mixture. If more than one type of plasmid is used to transfect host cells, each type may be present in nucleic acid in equal molar concentration, or in different stoichiometries.

In certain embodiments, nucleic acid (including but not limited to plasmid DNA, bacmid DNA, or other types of DNA or nucleic acid) comprise genes and/or other genetic information required to produce a recombinant viral vector, non-limiting examples of which include an adenoviral (AdV) vector, adeno-associated viral (AAV) vector, retroviral vector (such as gamma retroviral vectors derived from murine leukemia virus (MuLV)), or lentiviral vector (LV) (such as those derived from the human immunodeficiency viruses HIV-1 and HIV-2, simian immunodeficiency virus (SIV), feline immunodeficiency virus (FIV), bovine immunodeficiency virus, or caprine arthritis-encephalitis virus). As is known in the art, recombinant AAV vectors can be made in host cells by introducing into such cells, such as by transfection, genes that encode viral helper factors (such as those from adenovirus (AdV) or herpesvirus (HSV)), AAV Rep proteins, AAV capsid proteins, and a vector genome comprising AAV cis elements and a transgene, designed to be packaged into an AAV capsid. Similarly, LV vectors can be made in host cells by introducing into such cells, such as by transfection, genes encoding LV helper factor (such as gal, pol, and rev), heterologous viral envelope glycoproteins (such as VSV-g), and a transfer vector (such as the SIN transfer vector) containing a transgene and LV cis elements for packaging into the vector. LV vector production is described further in Merten, O-W, et al., Production of lentiviral vectors, Mol Ther Methods Clin Dev 3:16017, doi:10.1038/mtm.2016.17 (2016).

In some embodiments, the genes needed for production of a desired biological product in host cells including, without limitation, recombinant viral vectors, can be contained in 1, 2, 3, 4, or more types of plasmid for transfection. For example, as known in the art, recombinant AAV vectors are often produced using the so-called triple transfection technique, where genes for all viral (e.g., AdV or HSV) helper factors are contained in a first plasmid, AAV rep and AAV cap genes are together contained in a second plasmid, and the vector genome is contained in a third plasmid. This arrangement is not required however, and the necessary genes and other sequences could be contained on two or even just one plasmid. For example, all helper factors and the rep and cap genes could be contained in one plasmid, and the vector genome contained by a second, or all these genes and sequences could be contained in just one plasmid. Often, practical considerations guide the choice, since very large plasmids may be harder to produce in large quantities, and/or may be more sensitive to shear forces. In some embodiments, plasmids for recombinant AAV vector production can further include an origin of replication and an antibiotic resistance gene to facilitate growth in bacteria under antibiotic selection (for example, by adding to the bacterial culture medium ampicillin, kanamycin, or other antibiotics known in the art), a eukaryotic genetic control region, such as a promoter and optionally one or more enhancers for transcription of the genes in the transfected cells, transcription termination signal sequences (such as a polyadenylation signal sequence), and potentially other genetic sequences that facilitate efficient vector production in host cells. In some embodiments, one or more of the genes needed for recombinant AAV or LV (or other virus-derived) vector production can be produced by the host cells themselves and, in such embodiments, it is not necessary to supply that gene in a plasmid. For example, host cells can be stably transfected with genes to express a helper factor, Rep, capsid protein, or some of the genes required for LV vector production, to create so-called producer or packaging cell lines. Alternatively, the genome of host cells can be modified to express such genes constitutively or under the control of an inducible regulatory element.

The plasmid containing the AAV vector genome can, in some embodiments, include as part of the genome two AAV inverted terminal repeats (ITR), one positioned at each end of the genome sequence, a therapeutic transgene under the control of a genetic regulatory element, such as a promoter and optionally an enhancer to drive transcription in a transduced target cell, and a transcription termination signal sequence. AAV vector genomes can optionally include other sequences, such as an intron, stuffer sequence(s) (that may function solely by ensuring that the overall genome size is close to the packaging capacity of the capsid), a modified ITR to facilitate production of so-called self-complementary vectors (scAAV), as well as others known in the art.

### Host Cells

Methods and systems of the disclosure can be used for transfection of any suitable host cell. In some embodiments, host cells include any eukaryotic cells known in the art to be transfectable and capable of producing biological products from the genetic information introduced into the cells as a result of transfection. Host cells can be eukaryotic cells from different phyla, classes, orders, families, genera, or species. Non-limiting examples include plant, fungal, or animal cells. More specific non-limiting examples include yeast cells, insect cells, and mammalian cells. Mammalian cells can include human, ovine, porcine, murine, rat, bovine cells, or cells from other mammals. Host cells may be primary cells or cell lines that are capable of indefinite growth in culture. Examples of cell lines include HEK (human embryonic kidney) cells (such as HEK293 cells, or variants thereof, such as HEK 293E, HEK 293F, HEK 293H, HEK 293T, or HEK 293FT cells), Chinese hamster ovary (CHO) cells (such as CHO-K1, CHO-DXB11, CHO-DG44, CHO-S, CHOK1SV™, or CHOK1SV GS-KO™ cells), HeLa cells, HT1080 cells, COS cells (such as COS7 cells), VERO cells, PerC.6 cells, Sp2/0 cells, NS0 cells, NIH 3T3 cells, W138 cells, BHK cells, HEPG2 cells, A549 cells, C2C12 cells, H9C2 cells, HCT116 cells, HepG2 cells, HT-29 cells, Huh7 cells, Jurkat cells, K562 cells, LnCaP cells, MCF7 cells, PC-12 cells, PC-3 cells, RAW 264.7 cells, U2OS cells, C127 cells, AGE1.HN cells, CAP cells, HKB-11 cells, or MDCK cells, with others possible as well. Exemplary insect cells include without limitation Sf9 cells, Sf1 cells, Sf21 cells, Tn-368 cells, ExpiSf9 cells, D.Mel2 cells, BTI-Tn-5B1 cells, or BTI-Tn-5B1-4 cells, with others possible as well.

For production of recombinant AAV vectors, exemplary non-limiting host cells can include HEK293 cells (or variants thereof, such as HEK 293E, HEK 293F, HEK 293H, HEK 293T, or HEK 293FT cells), including HEK293 cells that are adapted to growth in suspension, and/or growth in the absence of serum or other animal products. Other cells for production of recombinant AAV vectors are possible, however, according to the knowledge of persons of ordinary skill in the art.

### Host Cell Culture Formats

The technology for growing and maintaining cells in culture, including at high volume and densities, is varied and familiar to those of ordinary skill in the art. Host cells may be grown in adherent cell culture or in suspension in culture in a variety of formats. As is common in industry, host cells are often grown in culture from working cell banks derived from master cell banks, but this convention should not be considered limiting.

In some embodiments, host cells may be grown in adherent cell culture in flasks, roller bottles, on hollow fibers, or in other formats known in the art. The cells can be transfected in the same container in which they are grown, or released from their substrate by chemical, enzymatic or other treatment and then transferred to a different vessel or container for transfection.

Host cells can also be grown in suspension, including at high volume and density, in special purpose vessels (often referred to as bioreactors) of a wide variety of sizes and formats familiar to those of ordinary skill in the art. Non-limiting examples include bottles, tanks (which can be open or closed to reduce contamination), and even large plastic bags with suitably thick walls. Bioreactors can be made of many materials, including stainless steel, glass, and plastics, and can be designed for multiple use or single use. Bioreactors may be designed with fluid inputs and outputs (such as with tubes and valves), and can be configured to permit temperature control, gas exchange and mixing of the contents, such as by stirring, mixing, or some other method of agitation, to maintain environmental conditions conducive to optimal cell growth, viability, and productivity. Cells grown in suspension can be transfected in the same container (e.g., bioreactor) in which they are grown, or transferred to a different vessel or container for transfection. In some embodiments, when the ultimate cell density and/or volume of cells to be transfected is large, cells from working cell banks may be grown in a series of containers of increasing size to expand their number before being transferred to a large volume bioreactor or other vessel or container for continued growth and/or transfection in accordance with the methods and systems of the disclosure. According to some embodiments, adherent cells can be grown on microcarriers suspended in a bioreactor, and transfected in the same vessel in similar fashion to cells grown in suspension.

Mixing of cells grown in suspension culture can be performed using any method or equipment known in the art. For example, in some embodiments, cells can be grown suspended in culture medium in a stirred tank bioreactor which is actively stirred by an impeller. Mixing can be performed at any suitable rate and/or power input per unit volume of media (P/V) in the bioreactor which, in some embodiments, can be expressed as watts per cubic meter (W/m³). Thus, for example, in some embodiments, mixing during the growth phase of cells in suspension culture can be performed such that the value of P/V is at least or about 5, 10, 15, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 95, or 100 W/m³, or more, or some other value between, or range comprising, any of the foregoing specifically enumerated P/V values. Mixing during cell growth can be at a constant rate or value of power input, or varied. Additional information about power input in stirred bioreactors can be found in, e.g., Kaiser, S C, et al., Power Input Measurements in Stirred Bioreactors at Laboratory Scale, J. Vis. Exp. (135), e56078, doi:10.3791/56078 (2018).

In the methods and systems described herein, transfection can occur in the same cell culture media in which host cells are grown, or the growth media can be removed and replaced (e.g., by perfusion) with a fresh supply of the same type of media, or of a different type of media, in which transfection is to occur. After addition of transfection cocktail, the same or a different type of media can be added to quench further transfection. After transfection, cells can be maintained in culture for a period of time to permit biosynthesis of a desired biological product. During this period media, whether the same or different as that in which the cells were grown and/or transfected, can also be exchanged (e.g., by perfusion) to maintain optimal conditions for continued cell viability and cellular synthesis of the desired biological product.

### Host Cell Culture Media

Host cells are grown in and, as noted above, can be transfected in culture media, an aqueous solution comprising all the macro and micronutrients required for cell growth and/or viability. As is well known, media recipes can be designed or modified to optimize growth and/or productivity of particular cell types and growth conditions. Media can be prepared from raw ingredients, but it is also possible to source pre-prepared media commercially in a variety of formats, such as powder or concentrated stocks. Media can also be supplemented with ingredients which contribute to optimal growth or production of particular biological products. For example, media can be supplemented with animal serum, such as fetal calf serum, although certain cells can be adapted to grow to high densities without added serum. Other non-limiting examples of media supplements include antibiotics, surfactants, growth factors, hormones, amino acids, glutamine, vitamins, salts, and metal ions required for certain enzymes to function properly.

Various classical media that are widely available (and can be customized if desired) for growth of certain mammalian cells include F17 Medium (also known by the proprietary name FreeStyle™ (Thermo-Fisher Scientific), Ham's F12 or F12K Medium, Dulbecco's Minimal Essential Medium (DMEM), RPMI 1640 Medium, DMEM/F12 Medium, Ham's F-10 Medium, Medium 199, Ames' Medium, BGJb Medium (Fitton-Jackson Modification), Click's Medium, CMRL-1066 Medium, Fischer's Medium, Glascow Minimum Essential Medium (GMEM), Iscove's Modified Dulbecco's Medium (IMDM), L-15 Medium (Leibovitz), McCoy's 5A Modified Medium, NCTC Medium, Swim's S-77 Medium, Waymouth Medium, and William's Medium E, with others being possible. Exemplary media for growth of certain insect cells include Express Five SFM, Sf-900 II SFM, Sf-900 III, or ExpiSf CD, with others possible.

### Host Cell Culture Volumes

The methods and systems of the disclosure can be used to transfect host cells grown or maintained in bioreactors or other vessels or containers at a variety of volumes (i.e., the combined volume of the cells themselves and the volume of the cell culture medium or other fluid in which the cells to be transfected are grown or suspended). Thus, in some embodiments, the cell suspension to which the transfection cocktail is added or delivered can have a volume ranging from about 1 liter (L) to 50000 L; 1 L to 10000 L; 2 L to 50000 L; 2 L to 10000 L; 5 L to 10000 L; 10 L to 10000 L; 20 L to 10000 L; 50 L to 10000 L; 100 L to 5000 L; 200 L to 5000 L; 200 L to 4000 L; 200 L to 3000 L; 500 L to 2500 L; 500 L to 2000 L; 1000 L to 2000 L; 750 L to 2000 L; 750 L to 1500 L; 800 L to 1400 L; 900 L to 1300 L; 1000 L to 1200 L; or at least or about 1, 10, 20, 30, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1600, 1700, 1800, 1900, 2000, 2500, 3000, 3500, 4000, 4500, 5000, 6000, 7000, 8000, 9000, 10000, 20000, 30000, 40000, or 50000 L, or more, or some other value between, or range comprising, any of the foregoing specifically enumerated values.

### Host Cell Density

The methods and systems of the disclosure can be used to transfect host cells at a variety of viable cell densities. Such cell densities can be achieved by growing the cells in culture (such as in suspension in a bioreactor) to a target viable cell density or range thereof, whereas in other embodiments a target cell density can be achieved by concentrating or diluting a sample of host cells as desired using media or other fluid compatible with transfection. Viability of cells in culture can be determined using any method known to those of ordinary skill in the art, for example, by taking a small sample of cells, adding a vital dye, such as trypan blue, and then counting the total number of cells excluding the dye on a hemocytometer, from which the number of viable cells per mL (or any other volume) can readily be calculated. Alternatively, viable cell density can be monitored during growth or maintenance in culture in real time using sensors, such as permittivity sensors, more information about which can be found, e.g., in Metze, S, et al., Monitoring online biomass with a capacitance sensor during scale-up of industrially relevant CHO cell culture fed-batch processes in single-use bioreactors, Bioprocess Biosys. Eng. 43:193-205 (2020). Other methods for quantifying viable cell density in a sample of cell culture will be familiar to those of ordinary skill in the art.

In some embodiments of the disclosure, the sample of host cells to which transfection cocktail is added or delivered at the start of transfection can have a viable cell density of at least or about 0.01×10⁶, 0.1×10⁶, 0.5×10⁶, 1×10⁶, 2×10⁶, 3×10⁶, 4×10⁶, 5×10⁶, 6×10⁶, 7×10⁶, 8×10⁶, 9×10⁶, 6, 11×10⁶, 12×10⁶, 13×10⁶, 14×10⁶, 15×10⁶, 16×10⁶, 17×10⁶, 18×10⁶, 19×10⁶, 20×10⁶, 21×10⁶, 22×10⁶, 23×10⁶, 24×10⁶, 25×10⁶, 26×10⁶, 27×10⁶, 28×10⁶, 29×10⁶, 30×10⁶, 35×10⁶, 40×10⁶, 45×10⁶, 50×10⁶, 55×10⁶, 60×10⁶, 65×10⁶, 70×10⁶, 75×10⁶, 80×10⁶, 85×10⁶, 90×10⁶, 95×10⁶, or 100×10⁶ viable cells per milliliter (vc/mL) of the fluid, such as cell culture medium, in which cells are suspended, or more, or some other value between, or range comprising, any of the foregoing specifically enumerated values. Thus, for example, in some embodiments, the viable cell density of host cells before transfection can range from about 0.01×10⁶ to 100×10⁶ vc/mL; 0.05×10⁶ to 6 vc/mL; 17×10⁶ to 19×10⁶ vc/mL; 10×10⁶ to 20×10⁶ vc/mL; 11×10⁶ to 20×10⁶ vc/mL; 12×10⁶ to 20×10⁶ vc/mL; 13×10⁶ to 20×10⁶ vc/mL; 14×10⁶ to 20×10⁶ vc/mL; 15×10⁶ to 20×10⁶ vc/mL; 16×10⁶ to 20×10⁶ vc/mL; 17×10⁶ to 20×10⁶ vc/mL; 18×10⁶ to 20×10⁶ vc/mL; 19×10⁶ to 20×10⁶ vc/mL; 10×10⁶ to 21×10⁶ vc/mL; 11×10⁶ to 21×10⁶ vc/mL; 12×10⁶ to 21×10⁶ vc/mL; 13×10⁶ to 21×10⁶ vc/mL; 14×10⁶ to 21×10⁶ vc/mL; 15×10⁶ to 21×10⁶ vc/mL; 16×10⁶ to 21×10⁶ vc/mL; 17×10⁶ to 21×10⁶ vc/mL; 18×10⁶ to 21×10⁶ vc/mL; 19×10⁶ to 21×10⁶ vc/mL; 20×10⁶ to 21×10⁶ vc/mL; 10×10⁶ to 22×10⁶ vc/mL; 11×10⁶ to 22×10⁶ vc/mL; 12×10⁶ to 22×10⁶ vc/mL; 13×10⁶ to 22×10⁶ vc/mL; 14×10⁶ to 22×10⁶ vc/mL; 15×10⁶ to 22×10⁶ vc/mL; 16×10⁶ to 22×10⁶ vc/mL; 17×10⁶ to 22×10⁶ vc/mL; 18×10⁶ to 22×10⁶ vc/mL; 19×10⁶ to 22×10⁶ vc/mL; 20×10⁶ to 22×10⁶ vc/mL; 21×10⁶ to 22×10⁶ vc/mL; 10×10⁶ to 23×10⁶ vc/mL; 11×10⁶ to 23×10⁶ vc/mL; 12×10⁶ to 23×10⁶ vc/mL; 13×10⁶ to 23×10⁶ vc/mL; 14×10⁶ to 23×10⁶ vc/mL; 15×10⁶ to 23×10⁶ vc/mL; 16×10⁶ to 23×10⁶ vc/mL; 17×10⁶ to 23×10⁶ vc/mL; 18×10⁶ to 23×10⁶ vc/mL; 19×10⁶ to 23×10⁶ vc/mL; 20×10⁶ to 23×10⁶ vc/mL; 21×10⁶ to 23×10⁶ vc/mL; 10×10⁶ to 24×10⁶ vc/mL; 11×10⁶ to 24×10⁶ vc/mL; 12×10⁶ to 24×10⁶ vc/mL; 13×10⁶ to 24×10⁶ vc/mL; 14×10⁶ to 24×10⁶ vc/mL; 15×10⁶ to 24×10⁶ vc/mL; 16×10⁶ to 24×10⁶ vc/mL; 17×10⁶ to 24×10⁶ vc/mL; 18×10⁶ to 24×10⁶ vc/mL; 19×10⁶ to 24×10⁶ vc/mL; 20×10⁶ to 24×10⁶ vc/mL; 21×10⁶ to 24×10⁶ vc/mL; 22×10⁶ to 24×10⁶ vc/mL; 23×10⁶ to 24×10⁶ vc/mL; 0.1×10⁶ to 25×10⁶ vc/mL; 0.25×10⁶ to 25×10⁶ vc/mL; 0.5×10⁶ to 25×10⁶ vc/mL; 1×10⁶ to 25×10⁶ vc/mL; 2×10⁶ to 25×10⁶ vc/mL; 2.5×10⁶ to 25×10⁶ vc/mL; 5×10⁶ to 25×10⁶ vc/mL; 6×10⁶ to 25×10⁶ vc/mL; 7×10⁶ to 25×10⁶ vc/mL; 8×10⁶ to 25×10⁶ vc/mL; 9×10⁶ to 25×10⁶ vc/mL; 6 to 25×10⁶ vc/mL; 11×10⁶ to 25×10⁶ vc/mL; 12×10⁶ to 25×10⁶ vc/mL; 13×10⁶ to 25×10⁶ vc/mL; 14×10⁶ to 25×10⁶ vc/mL; 15×10⁶ to 25×10⁶ vc/mL; 16×10⁶ to 25×10⁶ vc/mL; 17×10⁶ to 6 vc/mL; 18×10⁶ to 25×10⁶ vc/mL; 19×10⁶ to 25×10⁶ vc/mL; 20×10⁶ to 25×10⁶ vc/mL; 21×10⁶ to 25×10⁶ vc/mL; 22×10⁶ to 25×10⁶ vc/mL; 23×10⁶ to 25×10⁶ vc/mL; 24×10⁶ to 25×10⁶ vc/mL; 6 to 26×10⁶ vc/mL; 11×10⁶ to 26×10⁶ vc/mL; 12×10⁶ to 26×10⁶ vc/mL; 13×10⁶ to 26×10⁶ vc/mL; 14×10⁶ to 26×10⁶ vc/mL; 15×10⁶ to 26×10⁶ vc/mL; 16×10⁶ to 26×10⁶ vc/mL; 17×10⁶ to 26×10⁶ vc/mL; 18×10⁶ to 26×10⁶ vc/mL; 19×10⁶ to 26×10⁶ vc/mL; 20×10⁶ to 26×10⁶ vc/mL; 21×10⁶ to 26×10⁶ vc/mL; 22×10⁶ to 26×10⁶ vc/mL; 23×10⁶ to 26×10⁶ vc/mL; 24×10⁶ to 26×10⁶ vc/mL; 6 to 26×10⁶ vc/mL; 10×10⁶ to 27×10⁶ vc/mL; 11×10⁶ to 27×10⁶ vc/mL; 12×10⁶ to 27×10⁶ vc/mL; 13×10⁶ to 27×10⁶ vc/mL; 14×10⁶ to 27×10⁶ vc/mL; 15×10⁶ to 27×10⁶ vc/mL; 16×10⁶ to 27×10⁶ vc/mL; 17×10⁶ to 27×10⁶ vc/mL; 18×10⁶ to 27×10⁶ vc/mL; 19×10⁶ to 27×10⁶ vc/mL; 20×10⁶ to 27×10⁶ vc/mL; 21×10⁶ to 27×10⁶ vc/mL; 22×10⁶ to 27×10⁶ vc/mL; 23×10⁶ to 27×10⁶ vc/mL; 24×10⁶ to 27×10⁶ vc/mL; 25×10⁶ to 27×10⁶ vc/mL; 26×10⁶ to 27×10⁶ vc/mL; 10×10⁶ to 28×10⁶ vc/mL; 11×10⁶ to 28×10⁶ vc/mL; 12×10⁶ to 28×10⁶ vc/mL; 13×10⁶ to 28×10⁶ vc/mL; 14×10⁶ to 28×10⁶ vc/mL; 15×10⁶ to 28×10⁶ vc/mL; 16×10⁶ to 28×10⁶ vc/mL; 17×10⁶ to 28×10⁶ vc/mL; 18×10⁶ to 28×10⁶ vc/mL; 19×10⁶ to 28×10⁶ vc/mL; 20×10⁶ to 28×10⁶ vc/mL; 21×10⁶ to 28×10⁶ vc/mL; 22×10⁶ to 28×10⁶ vc/mL; 23×10⁶ to 28×10⁶ vc/mL; 24×10⁶ to 28×10⁶ vc/mL; 25×10⁶ to 28×10⁶ vc/mL; 26×10⁶ to 28×10⁶ vc/mL; 27×10⁶ to 28×10⁶ vc/mL; 10×10⁶ to 29×10⁶ vc/mL; 11×10⁶ to 29×10⁶ vc/mL; 12×10⁶ to 29×10⁶ vc/mL; 13×10⁶ to 29×10⁶ vc/mL; 14×10⁶ to 29×10⁶ vc/mL; 15×10⁶ to 29×10⁶ vc/mL; 16×10⁶ to 29×10⁶ vc/mL; 17×10⁶ to 29×10⁶ vc/mL; 18×10⁶ to 29×10⁶ vc/mL; 19×10⁶ to 29×10⁶ vc/mL; 20×10⁶ to 29×10⁶ vc/mL; 21×10⁶ to 29×10⁶ vc/mL; 22×10⁶ to 29×10⁶ vc/mL; 23×10⁶ to 29×10⁶ vc/mL; 24×10⁶ to 29×10⁶ vc/mL; 25×10⁶ to 29×10⁶ vc/mL; 26×10⁶ to 29×10⁶ vc/mL; 27×10⁶ to 29×10⁶ vc/mL; 28×10⁶ to 29×10⁶ vc/mL; 2×10⁶ to 30×10⁶ vc/mL; 5×10⁶ to 6 vc/mL; 10×10⁶ to 30×10⁶ vc/mL; 11×10⁶ to 30×10⁶ vc/mL; 12×10⁶ to 30×10⁶ vc/mL; 13×10⁶ to 30×10⁶ vc/mL; 14×10⁶ to 30×10⁶ vc/mL; 15×10⁶ to 30×10⁶ vc/mL; 16×10⁶ to 30×10⁶ vc/mL; 17×10⁶ to 30×10⁶ vc/mL; 18×10⁶ to 30×10⁶ vc/mL; 19×10⁶ to 30×10⁶ vc/mL; 20×10⁶ to 30×10⁶ vc/mL; 21×10⁶ to 30×10⁶ vc/mL; 22×10⁶ to 30×10⁶ vc/mL; 23×10⁶ to 30×10⁶ vc/mL; 24×10⁶ to 6 vc/mL; 25×10⁶ to 30×10⁶ vc/mL; 26×10⁶ to 30×10⁶ vc/mL; 27×10⁶ to 30×10⁶ vc/mL; 28×10⁶ to 30×10⁶ vc/mL; 29×10⁶ to 30×10⁶ vc/mL, 10×10⁶ to 35×10⁶ vc/mL; 11×10⁶ to 35×10⁶ vc/mL; 12×10⁶ to 35×10⁶ vc/mL; 13×10⁶ to 35×10⁶ vc/mL; 14×10⁶ to 35×10⁶ vc/mL; 15×10⁶ to 35×10⁶ vc/mL; 16×10⁶ to 35×10⁶ vc/mL; 17×10⁶ to 35×10⁶ vc/mL; 18×10⁶ to 35×10⁶ vc/mL; 19×10⁶ to 6 vc/mL; 20×10⁶ to 35×10⁶ vc/mL; 21×10⁶ to 35×10⁶ vc/mL; 22×10⁶ to 35×10⁶ vc/mL; 23×10⁶ to 35×10⁶ vc/mL; 24×10⁶ to 35×10⁶ vc/mL; 25×10⁶ to 35×10⁶ vc/mL; 26×10⁶ to 35×10⁶ vc/mL; 27×10⁶ to 35×10⁶ vc/mL; 28×10⁶ to 35×10⁶ vc/mL; 29×10⁶ to 35×10⁶ vc/mL, 30×10⁶ to 35×10⁶ vc/mL, 31×10⁶ to 35×10⁶ vc/mL, 32×10⁶ to 35×10⁶ vc/mL, 33×10⁶ to 35×10⁶ vc/mL, 34×10⁶ to 6 vc/mL, 10×10⁶ to 40×10⁶ vc/mL; 11×10⁶ to 40×10⁶ vc/mL; 12×10⁶ to 40×10⁶ vc/mL; 13×10⁶ to 40×10⁶ vc/mL; 14×10⁶ to 40×10⁶ vc/mL; 15×10⁶ to 40×10⁶ vc/mL; 16×10⁶ to 40×10⁶ vc/mL; 17×10⁶ to 40×10⁶ vc/mL; 18×10⁶ to 40×10⁶ vc/mL; 19×10⁶ to 40×10⁶ vc/mL; 20×10⁶ to 40×10⁶ vc/mL; 21×10⁶ to 40×10⁶ vc/mL; 22×10⁶ to 40×10⁶ vc/mL; 23×10⁶ to 40×10⁶ vc/mL; 24×10⁶ to 6 vc/mL; 25×10⁶ to 40×10⁶ vc/mL; 26×10⁶ to 40×10⁶ vc/mL; 27×10⁶ to 40×10⁶ vc/mL; 28×10⁶ to 40×10⁶ vc/mL; 29×10⁶ to 40×10⁶ vc/mL, 30×10⁶ to 40×10⁶ vc/mL, 31×10⁶ to 40×10⁶ vc/mL, 32×10⁶ to 40×10⁶ vc/mL, 33×10⁶ to 40×10⁶ vc/mL, 34×10⁶ to 40×10⁶ vc/mL, 35×10⁶ to 40×10⁶ vc/mL, 36×10⁶ to 40×10⁶ vc/mL, 37×10⁶ to 40×10⁶ vc/mL, 38×10⁶ to 40×10⁶ vc/mL, or 39×10⁶ to 40×10⁶ vc/mL, or some other range. In some embodiments, the host cells are HEK293 cells, or variants thereof, in suspension culture.

### Concentrations, Volumes and Ratios for Transfection

Transfection reagent solutions (including, but not limited to that containing PEI) for use in the methods and systems of the disclosure can be prepared at any suitable concentration of transfection reagent (including, but not limited to PEI), including at least or about 0.001, 0.005, 0.05, 0.1, 0.5, 1, 1.1, 1.2, 1.3, 1.4, 1.5, 1.6, 1.7, 1.8, 1.9, 2, 2.1, 2.2, 2.3, 2.4, 2.5, 5, 7.5, 10, or 50 milligrams, or more, transfection reagent (including, but not limited to PEI) per milliliter (mg/mL) of the solvent or diluent in which the transfection reagent is dissolved or diluted, or more, or some other value between or range comprising any of the foregoing specifically enumerated values. In other embodiments, the concentration of transfection reagent in the transfection reagent solution for use in the methods and systems of the disclosure can be at least or about 0.001, 0.005, 0.01, 0.05, 0.1, 0.5, 1, 1.5, 2, 2.5, 5, 7.5, 10, 20, 50, 500 mM, or more, or some other value between or range comprising any of the foregoing specifically enumerated values.

Nucleic acid solutions (including, but not limited to that containing plasmid DNA) for use in the methods and systems of the disclosure can be prepared at any suitable concentration of nucleic acid (including, but not limited to pDNA), including at least or about 0.001, 0.005, 0.01, 0.1, 0.5, 0.6, 0.7, 0.8, 0.9, 1, 1.5, 2, 2.5, 5, 7.5, 10, 20, 50 mg, or more, nucleic acid per mL of solvent or diluent in which the nucleic acid (including, but not limited to pDNA) is dissolved or diluted, or more, or some other value between or range comprising any of the foregoing specifically enumerated values. In other embodiments, the concentration of nucleic acid in the nucleic acid solution for use in the methods and systems of the disclosure can be at least or about 0.005, 0.01, 0.05, 0.1, 0.5, 1, 1.5, 2, 2.5, 5, 7.5, 10, 20, 50, 500 mM, or more, or some other value between or range comprising any of the foregoing specifically enumerated values.

As noted above, any biocompatible solvent or diluent known in the art to support complexation of the chosen transfection reagent and nucleic acid can be used in preparing transfection reagent solution and nucleic acid solution, non-limiting examples of which include saline, phosphate-buffered saline, dextrose solution, Ringer's lactate solution, cell growth media (e.g., F17 medium), or water. In addition, in some embodiments, such solvents and diluents can further comprise other ingredients, such as salts, buffers, or detergents, a non-limiting example of which is pluronic, such as pluronic at a concentration of 0.2%.

In nucleic acid solutions or transfection cocktail containing more than one type of nucleic acid, for example, different DNA plasmids containing non-identical nucleotide sequences, the different types of nucleic acid can be present at different molar ratios. Thus, for example, in some embodiments, in a nucleic acid solution or transfection cocktail comprising at least two types of plasmids, any two such types of plasmids can be present in a molar ratio of about 50:1 to about 1:50, 20:1 to about 1:20, 10:1 to about 1:10, 9:1 to about 1:9, 8:1 to about 1:8, 7:1 to about 1:7, 6:1 to about 1:6, 5:1 to about 1:5, 4:1 to about 1:4, or 3:1 to about 1:3, or any ratios encompassed by these ranges, including for example, about 3:1, 2.9:1, 2.8:1, 2.7:1, 2.6:1, 2.5:1, 2.4:1, 2.3:1, 2.2:1, 2.1:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, or 1:3, or some other ratio between or range of ratios comprising any of the foregoing specifically enumerated ratios, others also being possible, where the first (antecedent) and second (consequent) numbers in the ratio respectively represent the relative amount of moles or molar concentration of the first and second types of plasmid in the nucleic acid solution or transfection cocktail. In some embodiments the molar ratio of a first and a second type of DNA plasmids in the nucleic acid solution or transfection cocktail is about 1:1, with a deviation of either value not exceeding±50%, ±40%, ±30%, ±20%, ±10%, or ±5%. In certain exemplary non-limiting embodiments, the first plasmid type comprises genes for adenovirus helper factors and/or AAV Rep and AAV capsid proteins, and the second plasmid type comprises an AAV vector genome comprising a gene under the control of a genetic regulatory element (such as a promoter and optionally an enhancer) as well as at least one AAV inverted terminal repeat.

In some other non-limiting embodiments, in a nucleic acid solution or transfection cocktail comprising at least three types of plasmids, any three such types of plasmids can be present in molar ratios of about 1:1:1, 1:1:2, 1:1:3, 1:2:1, 1:2:2, 1:2:3, 1:3:1, 1:3:2, 1:3:3, 2:1:1, 2:1:2, 2:1:3, 2:2:1, 2:2:2, 2:2:3, 2:3:1, 2:3:2, 2:3:3, 3:1:1, 3:1:2, 3:1:3, 3:2:1, 3:2:2, 3:2:3, 3:3:1, 3:3:2, 3:3:3, 1:2:2, 1:2:3, or 1:3:3, or some other ratio between or range of ratios comprising any of the foregoing specifically enumerated ratios, others also being possible, where the first, second, and third numbers in the ratios respectively represent the relative amount of moles or molar concentration of the first, second, and third types of plasmid in the nucleic acid solution or transfection cocktail. In some embodiments, the relative molar concentrations of the three plasmids is about 1:1:1, 1:1:2, 1:1:3, 1:2:1, 1:2:2, 1:2:3, 1:3:1, 1:3:2, 1:3:3, 2:1:1, 2:1:2, 2:1:3, 2:2:1, 2:2:2, 2:2:3, 2:3:1, 2:3:2, 2:3:3, 3:1:1, 3:1:2, 3:1:3, 3:2:1, 3:2:2, 3:2:3, 3:3:1, 3:3:2, 3:3:3, 1:2:2, 1:2:3, or 1:3:3, with a deviation of the first, second or third values not exceeding±50%, ±40%, ±30%, ±20%, ±10%, or ±5%. In certain exemplary non-limiting embodiments, the first plasmid type comprises genes for adenovirus helper factors, the second plasmid type comprises genes encoding AAV Rep and AAV capsid proteins, and the third plasmid type comprises an AAV vector genome comprising a gene under the control of a genetic regulatory element (such as a promoter and optionally an enhancer), as well as at least one AAV inverted terminal repeat.

Transfection reagent solution and nucleic acid solution for use in the methods and systems of the disclosure may be prepared separately in any suitable amounts, which may be expressed as a volume or mass. In some embodiments the volume or mass of transfection reagent solution that is prepared (including, but not limited to that of PEI) ranges from about 0.1 to 5000 liters (L) or kilograms (kg), or more, or at least or about 0.1, 0.5, 1, 5, 10, 20, 30, 40, 50, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 600, 700, 800, 900, 1000, 1500, 2000, 2500, 3000, 3500, 4000, 4500, or 5000 L or kg, or more, or some other value between or range comprising any of the foregoing specifically enumerated values. In some embodiments the volume or mass of nucleic acid solution (including, but not limited to that of pDNA) that is prepared ranges from about 0.1 to 5000 L or kg, or more, or at least or about 0.1, 0.5, 1, 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 600, 700, 800, 900, 1000, 1500, 2000, 2500, 3000, 3500, 4000, 4500, or 5000 L or kg, or more, or some other value between or range comprising any of the foregoing specifically enumerated values.

Transfection cocktail for use in the methods and systems of the disclosure can be prepared in any suitable amount, all or a portion of which is ultimately to be delivered or added to a sample of cells to be transfected, and may be expressed as a volume or mass. In some embodiments the total volume or mass of transfection cocktail that is prepared by mixing together transfection reagent solution (including, but not limited to that containing PEI) and nucleic acid solution (including, but not limited to that containing pDNA) ranges from about 0.1 to 10000 L or kg, or at least or about 0.1, 0.2, 0.5, 1, 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 950, 900, 950, 1000, 1500, 2000, 2500, 3000, 3500, 4000, 4500, 5000, 5500, 6000, 6500, 7000, 7500, 8000, 8500, 9000, 9500, or 10000 L or kg, or more, or some other value between or range comprising any of the foregoing specifically enumerated values. As noted above, in some embodiments, the total volume or mass of transfection cocktail for use in transfection can be prepared as a bolus, or instead formed continuously over a period while at the same time a portion of transfection cocktail is being added or delivered to a sample of cells for transfection.

Transfection cocktail for use in the methods and systems of the disclosure can be delivered or added to a sample of cells to be transfected in any suitable amount, which may be expressed as a volume or mass. In some embodiments the total volume or mass of transfection cocktail (including but not limited to that containing PEI and pDNA) that is delivered or added to cells for transfection ranges from about 0.1 to 10000 L or kg, or at least or about 0.1, 0.2, 0.5, 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 950, 900, 950, 1000, 1500, 2000, 2500, 3000, 3500, 4000, 4500, 5000, 5500, 6000, 6500, 7000, 7500, 8000, 8500, 9000, 9500, or 10000 L or kg, or more, or some other value between or range comprising any of the foregoing specifically enumerated values. As noted above, in some embodiments, the total volume or mass of transfection cocktail for use in transfection can be delivered or added to cells as a bolus, or instead delivered or added to cells continuously over a period while at the same time transfection cocktail is being formed by mixing together transfection reagent solution and nucleic acid solution.

Once prepared, transfection reagent solution and nucleic acid solution for use in the methods and systems of the disclosure can be mixed together in any suitable volumetric or mass ratios to form transfection cocktail. In some embodiments transfection reagent solution (including, but not limited to that containing PEI) and nucleic acid solution (including, but not limited to that containing pDNA) can be combined to form transfection cocktail in ratios of, for example, about 50:1 to about 1:50, 20:1 to about 1:20, 10:1 to about 1:10, 9:1 to about 1:9, 8:1 to about 1:8, 7:1 to about 1:7, 6:1 to about 1:6, 5:1 to about 1:5, 4:1 to about 1:4, or 3:1 to about 1:3, or any ratios encompassed by these ranges, including for example, about 9:1, 8:1, 7:1, 6:1, 4:1, 3:1, 2:1, 1:1, 1:2, 1:3, 1:4, 1:5, 1:6, 1:7, 1:8, 1:9, or some other ratio between or range of ratios comprising any of the foregoing specifically enumerated ratios, others also being possible, wherein the first and second numbers respectively indicate the relative amounts of transfection reagent solution and nucleic acid solution that are combined, on a volume (e.g., liters) or mass (e.g., kilograms) basis. In some embodiments, transfection reagent solution and nucleic acid solution are combined in a ratio of approximately 1:1, on a volume or mass basis. In some embodiments, systems of the disclosure can be configured to effect mixing of the desired volume ratios, for example, by setting pump means to operate at different pump rates where different amounts of transfection reagent solution and nucleic acid solution are desired to be mixed in a period of time. In some embodiments, the total volumes of transfection reagent solution and nucleic acid solution that are prepared are combined to form transfection cocktail for transfection of cells, whereas in other embodiments less than the total volumes of such solutions are combined.

Transfection cocktail for use in the methods and systems of the disclosure can include transfection reagent (including, but not limited to PEI) and nucleic acid (including, but not limited to pDNA) in any suitable concentration. In some embodiments, transfection cocktail can contain transfection reagent (including, but not limited to PEI) at concentration of at least or about 0.001, 0.01, 0.05, 0.1, 0.5, 1, 1.1, 1.2, 1.3, 1.4, 1.5, 1.6, 1.7, 1.8, 1.9, 2, 2.1, 2.2, 2.3, 2.4, 2.5, 5, 7.5, 20, or 50 mg/mL, or more, or some other value between or range comprising any of the foregoing specifically enumerated values. In other embodiments, the concentration of transfection reagent in the transfection cocktail can be at least or about 0.001, 0.005, 0.01, 0.05, 0.5, 1, 1.5, 2, 2.5, 5, 7.5, 10, 20, 50, 500 mM, or more, or some other value between or range comprising any of the foregoing specifically enumerated values. In some embodiments, transfection cocktail can contain nucleic acid (including, but not limited to pDNA) at concentration of at least or about 0.001, 0.005, 0.01, 0.05, 0.1, 0.5, 0.6, 0.7, 0.8, 0.9, 1, 1.5, 2, 2.5, 7.5, 10, 20, 50 mg/mL, or more, or some other value between or range comprising any of the foregoing specifically enumerated values. In other embodiments, the concentration of nucleic acid in the transfection cocktail can be at least or about 0.001, 0.005, 0.01, 0.05, 0.1, 0.5, 1, 1.5, 2, 2.5, 5, 7.5, 10, 20, 50, 500 mM, or more, or some other value between or range comprising any of the foregoing specifically enumerated values.

Transfection cocktail for use in the methods and systems of the disclosure can include transfection reagent (including, but not limited to PEI) and nucleic acid (including, but not limited to pDNA) in any suitable mass ratios. In some embodiments the ratio of the mass of transfection reagent (including, but not limited to PEI) to the mass of nucleic acid (including, but not limited to pDNA) in transfection cocktail can range from about 100:1 to about 1:100, about 50:1 to about 1:50, about 20:1 to about 1:20, or about 10:1 to about 1:10, or any ratios encompassed by these ranges, including for example, about 9:1, 8:1, 7:1, 6:1, 5:1, 4:1, 3:1, 2.9:1, 2.8:1, 2.7:1, 2.6:1, 2.5:1, 2.4:1, 2.3:1, 2.2:1, 2.1:1, 2:1, 1:1, 1:2, 1:3, 1:4, 1:5, 1:6, 1:7, 1:8, 1:9, or some other ratio between or range of ratios comprising any of the foregoing specifically enumerated ratios, others also being possible, wherein the first and second numbers respectively indicate the relative amounts of transfection reagent and nucleic acid in transfection cocktail on a mass (e.g., grams or milligrams) basis.

In some embodiments, the transfection reagent is a polycationic polymer comprising a plurality of primary, secondary, and/or tertiary amine groups, non-limiting examples of which include PEI, such as linear PEI or branched PEI. As is known in the art, the molar concentration of nitrogen atoms in the amine groups in a solution of the polymer can be calculated, as can the molar concentration of phosphorus atoms in the phosphate groups in a solution of a nucleic acid. Once the molar concentrations of amines and phosphates in the respective stock solutions of transfection reagent and nucleic acid is known, the molar ratio of the number of nitrogen atoms to the number of phosphorus atoms when transfection reagent and nucleic acid solutions are combined into transfection cocktail can also be calculated and expressed as the N/P ratio. As known in the art, the N/P ratio can be varied, which has been shown to have an effect on transfection efficiency. See, e.g., Boussif, 0, et al., A versatile vector for gene and oligonucleotide transfer into cells in culture and in vivo: Polyethylenimine, PNAS 92:7297-7301 (1995). Transfection cocktail for use in the methods and systems of the disclosure can include any desired N/P ratio. Thus, for example, in some embodiments, the N/P ratio of transfection cocktail comprising a polycationic polymer, such as PEI, and a nucleic acid, such as pDNA, can be at least or about 0.01, 0.05, 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 250, 300, 350, 400, 450, or 500, or more, or some other ratio between or range of ratios comprising any of the foregoing specifically enumerated N/P ratios.

Methods of the disclosure can be performed such that any suitable amount of transfection reagent and nucleic acid are used to transfect cells. In some embodiments, the amount of transfection reagent and nucleic acid used to transfect cells can be expressed as a ratio of their amounts relative to a certain number of viable cells to be transfected. For example, amounts of transfection reagent and nucleic acid used in transfections can be expressed in micrograms per million viable cells. Thus, in some embodiments, the ratio of the mass of transfection reagent (including, but not limited to PEI) to million viable cells to be transfected can range from about 0.1 to 50 μg per 1×10⁶ viable cells; 0.5 to 30 μg per 1×10⁶ viable cells; 0.75 to μg per 1×10⁶ viable cells; 1 to 3 μg per 1×10⁶ viable cells; or about 1.65 μg per 1×10⁶ viable cells, or can be at least or about 0.1, 0.2, 0.25, 0.3, 0.4, 0.5, 0.6, 0.65, 0.7, 0.75, 0.8, 0.9, 1, 1.1, 1.2, 1.3, 1.4, 1.5, 1.6, 1.65, 1.7, 1.8, 1.9, 2, 3, 4, 5, 6, 7, 8, 9, 10, 15, 20, 25, 30, 35, 40, 45, or 50 μg per 1×10⁶ viable cells, or more, or some other value between, or range comprising, any of the foregoing specifically enumerated values. Likewise, in some embodiments, the ratio of the mass of nucleic acid (including, but not limited to pDNA) to million viable cells to be transfected can range from about 0.05 to 20 μg per 1×10⁶ viable cells; 0.1 to 10 μg per 1×10⁶ viable cells; 0.25 to 7.5 μg per 1×10⁶ viable cells; 0.5 to 5 μg per 1×10⁶ viable cells; 0.5 to 2.5 μg per 1×10⁶ viable cells; to 1.0 μg per 1×10⁶ viable cells, or is about 0.75 μg per 1×10⁶ viable cells, or can be at least or about 0.1, 0.2, 0.3, 0.4, 0.5, 0.6, 0.65, 0.7, 0.75, 0.8, 0.85, 0.9, 1, 2, 3, 4, 5, 6, 7, 8, 9, or 10 μg per 1×10⁶ viable cells, or more, or some other value between, or range comprising, any of the foregoing specifically enumerated values. Knowing the approximate total number of viable cells to be transfected, the concentration of transfection reagent in, and/or the amount of transfection reagent solution used in a transfection can be controlled to deliver an amount of transfection reagent to cells to be transfected sufficient to achieve the desired transfection reagent mass to cell number ratio. Similarly, the concentration of nucleic acid in, and/or the amount of nucleic acid solution used in a transfection can be controlled to deliver an amount of nucleic acid to cells to be transfected sufficient to achieve the desired nucleic acid mass to cell number ratio.

In other embodiments, the amount of transfection reagent and nucleic acid used to transfect cells can be expressed as a ratio of their amounts relative to a certain volume of a sample of cells to be transfected. For example, amounts of transfection reagent and nucleic acid used in transfections can be expressed in micrograms per milliliter of cells suspended in a fluid (e.g., cell growth media) in which they are to be transfected. Thus, in some embodiments, the ratio of the mass of transfection reagent (including, but not limited to PEI) to mL of cell sample to be transfected can range from about 0.1 to 50 μg/mL; 0.5 to 30 μg/mL; 0.75 to 10 μg/mL; 1 to 3 μg/mL; or about 1.65 μg/mL, or can be at least or about 0.5, 0.6, 0.65, 0.7, 0.75, 0.8, 0.9, 1, 1.1, 1.2, 1.3, 1.4, 1.5, 1.6, 1.65, 1.7, 1.8, 1.9, 2, 3, 4, 5, 6, 7, 8, 9, 10, 15, 20, 25, 30, 35, 40, 45, or 50 μg/mL, or more, or some other value between, or range comprising, any of the foregoing specifically enumerated values. Likewise, in some embodiments, the ratio of the mass of nucleic acid (including, but not limited to pDNA) to mL of cell sample to be transfected can range from about 0.05 to 20 μg/mL; 0.1 to 10 μg/mL; 0.25 to 7.5 μg/mL; 0.5 to 5 μg/mL; 0.5 to 2.5 μg/mL; to 1.0 μg/mL, or is about 0.75 μg/mL, or can be at least or about 0.1, 0.2, 0.3, 0.4, 0.5, 0.6, 0.7, 0.75, 0.8, 0.85, 0.9, 1, 2, 3, 4, 5, 6, 7, 8, 9, or 10 μg/mL, or more, or some other value between, or range comprising, any of the foregoing specifically enumerated values. Knowing the approximate total volume of cell suspension to be transfected, the concentration of transfection reagent in, and/or the amount of transfection reagent solution used in a transfection can be controlled to deliver an amount of transfection reagent to cells to be transfected sufficient to achieve the desired transfection reagent mass to volume ratio. Similarly, the concentration of nucleic acid in, and/or the amount of nucleic acid solution used in a transfection can be controlled to deliver an amount of nucleic acid to cells to be transfected sufficient to achieve the desired nucleic acid mass to volume ratio.

Transfection cocktail for use in the methods and systems of the disclosure (including, but not limited to that containing PEI and pDNA) can be delivered or added to a sample of cells for transfection in any suitable amount. In some embodiments, the amount of transfection cocktail to be added to a sample of cells for transfection can be expressed as a percentage, on a weight by weight (w/w), weight by volume (w/v), or volume by volume (v/v) basis, of the amount of the cell sample to be transfected. Thus, for example, in some embodiments, the amount of transfection cocktail delivered or added to a cell sample for transfection can be at least or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 percent, or more, of the amount of the cell sample (e.g., as a suspension in a fluid, such as cell growth media, in which they are to be transfected) on a w/w, w/v, or v/v basis, or some other value between, or range comprising, any of the foregoing specifically enumerated values. In an exemplary non-limiting embodiment, the amount of transfection cocktail that can be added to a cell sample is 32.65% (w/v) of the cell sample volume.

Incubation time of the transfection cocktail can be any suitable period that provides sufficient time for transfection reagent and nucleic acid in suspension or solution to form complexes of transfection reagent and nucleic acid (including but not limited to PEI/pDNA complexes) that are capable of transfecting host cells with high efficiency. The incubation time period begins when a portion of transfection reagent solution and a portion of nucleic acid solution first contact each other and ends when the transfection cocktail so formed is delivered or added to a sample of cells for transfection. With reference to systems of the disclosure for transfection, incubation time in some embodiments is the time required for transfection cocktail to fluidly communicate from mixing means to cell containment means (for example, in a non-limiting embodiment, be pumped from a static in-line mixer into a bioreactor containing cells in culture through a tube connecting the mixer and bioreactor). In some embodiments, incubation time of the transfection cocktail can be at least or about 5, 10, 15, 20, 25, 30, 35, 40, 45, 50, 55, 65, 70, 75, 80, 85, 90, 95, 100, 105, 110, 115, 120, 125, 130, 135, 140, 145, 150, 160, 170, 180, 190, 200, 210, 220, 230, 240, 250, 260, 270, 280, 290, 300, 350, 400, 450, 500, 550, 600, 650, 700, 750, 800, 850, or 900 seconds, or more, or some other value between, or range comprising, any of the foregoing specifically enumerated values of time. In other embodiments, incubation time can be about 900 seconds or less, such as about 900, 850, 800, 750, 700, 650, 600, 550, 500, 450, 400, 350, 300, 290, 280, 270, 260, 250, 240, 230, 220, 210, 200, 190, 180, 170, 160, 155, 150, 145, 140, 135, 130, 125, 120, 115, 110, 100, 95, 90, 85, 80, 75, 70, 65, 60, 55, 50, 45, 40, 35, 30, 25, 20, 10, or 5 seconds, or less time, or some other value between, or range comprising, any of the foregoing specifically enumerated values of time.

Addition time of the transfection cocktail can be any suitable period sufficient for a predetermined volume or mass of transfection cocktail (including, but not limited to that containing PEI and pDNA) to be delivered or added to a sample of cells for transfection. In some embodiments, the predetermined volume or mass of transfection cocktail is the total volume or mass of transfection cocktail which has been prepared for purposes of transfection, or some portion thereof. In some embodiments, the predetermined volume or mass of transfection cocktail is at least or about 5, 6, 7, 8, 9, 10, 11, 12, 13, 14, 15, 16, 17, 18, 19, 20, 21, 22, 23, 24, 26, 27, 28, 29, 30, 31, 32, 33, 34, 35, 36, 37, 38, 39, 40, 41, 42, 43, 44, or 45 percent, or more, of the volume or mass of the cell sample to be transfected. With reference to systems of the disclosure for transfection, addition time in some embodiments is the time required for predetermined volumes or masses of transfection reagent solution (including but not limited to that containing PEI) and nucleic acid solution (including but not limited to that containing pDNA) to fluidly communicate from solution containment means into mixing means, and therefrom to cell containment means. According to an exemplary non-limiting embodiment, addition time can be the time required for predetermined volumes or masses of transfection reagent solution (including but not limited to that containing PEI) and nucleic acid solution (including but not limited to that containing pDNA) to be pumped from their containers through tubes into a static in-line mixer (where they start to mix to form transfection cocktail), and then from the mixer through another tube into a bioreactor containing cells to be transfected. In some embodiments, the predetermined volumes or masses of transfection reagent solution and nucleic acid solution are the total volumes or masses of such solutions prepared for purposes of transfection, or some portion thereof.

In some embodiments, addition time of the transfection cocktail can be at least or about 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 85, 90, 95, 100, 105, 110, 115, 120, 125, 130, 135, 140, 145, 150, 160, 170 or 180 minutes, or more, or some other value between, or range comprising, any of the foregoing specifically enumerated values. In other embodiments, addition time can be about can be 180 minutes or less, such as about 180, 170, 160, 150, 145, 140, 135, 130, 125, 120, 115, 110, 100, 95, 90, 85, 80, 70, 65, 60, 55, 50, 45, 40, 35, 34, 33, 32, 31, 30, 29, 28, 27, 26, 25, 24, 23, 22, 21, 20, 19, 18, 17, 16, 15, 14, 13, 12, 11, 10, 9.5, 9, 8.5, 8, 7.5, 7, 6.5, 6, 5.5, 5, 4.5, 4, 3.5, 3, 2.5, 2, 1.5, or 1 minute, or less time, or some other value between, or range comprising, any of the foregoing specifically enumerated values of time.

In some exemplary, non-limiting embodiments, methods and systems of the disclosure can be performed and configured using incubation times and addition times that range approximately as set forth in Table 1. In other embodiments, the values in Table 1 can vary by ±30%, ±25, ±20%, ±15, ±10%, or ±5%.

TABLE 1

| Incubation Time | Addition Time |
|---|---|
| 30 to 180 seconds | 15 to 90 minutes |
| 60 to 150 seconds | 15 to 90 minutes |
| 75 to 150 seconds | 15 to 90 minutes |
| 85 to 140 seconds | 15 to 90 minutes |
| 30 seconds | 15 to 90 minutes |
| 45 seconds | 15 to 90 minutes |
| 60 seconds | 15 to 90 minutes |
| 90 seconds | 15 to 90 minutes |
| 120 seconds | 15 to 90 minutes |
| 135 seconds | 15 to 90 minutes |
| 30 to 180 seconds | 15 to 60 minutes |
| 60 to 150 seconds | 15 to 60 minutes |
| 75 to 150 seconds | 15 to 60 minutes |
| 85 to 140 seconds | 15 to 60 minutes |
| 30 seconds | 15 to 60 minutes |
| 45 seconds | 15 to 60 minutes |
| 60 seconds | 15 to 60 minutes |
| 90 seconds | 15 to 60 minutes |
| 120 seconds | 15 to 60 minutes |
| 135 seconds | 15 to 60 minutes |
| 30 to 180 seconds | 25 to 50 minutes |
| 60 to 150 seconds | 25 to 50 minutes |
| 75 to 150 seconds | 25 to 50 minutes |
| 85 to 140 seconds | 25 to 50 minutes |
| 30 seconds | 25 to 50 minutes |
| 45 seconds | 25 to 50 minutes |
| 60 seconds | 25 to 50 minutes |
| 90 seconds | 25 to 50 minutes |
| 120 seconds | 25 to 50 minutes |
| 135 seconds | 25 to 50 minutes |
| 30 to 180 seconds | 30 to 45 minutes |
| 60 to 150 seconds | 30 to 45 minutes |
| 75 to 150 seconds | 30 to 45 minutes |
| 85 to 140 seconds | 30 to 45 minutes |
| 30 seconds | 30 to 45 minutes |
| 45 seconds | 30 to 45 minutes |
| 60 seconds | 30 to 45 minutes |
| 90 seconds | 30 to 45 minutes |
| 120 seconds | 30 to 45 minutes |
| 135 seconds | 30 to 45 minutes |
| 30 to 180 seconds | 45 minutes |
| 60 to 150 seconds | 45 minutes |
| 75 to 150 seconds | 45 minutes |
| 85 to 140 seconds | 45 minutes |
| 30 seconds | 45 minutes |
| 45 seconds | 45 minutes |
| 60 seconds | 45 minutes |
| 90 seconds | 45 minutes |
| 120 seconds | 45 minutes |
| 135 seconds | 45 minutes |

In some embodiments, the sample of cells to be transfected can be stirred, agitated, or mixed during the delivery or addition of the transfection cocktail to the cells to effect thorough distribution of the transfection cocktail and mixing with the sample, and to prevent locally high concentrations of transfection cocktail from forming which might negatively impact cell viability. During mixing, environmental factors, such as temperature, pH and oxygenation, can be controlled within acceptable ranges. In some embodiments, mixing can occur during the entire period in which transfection cocktail is added, or for a portion of such time, such as at least or about 10%, 20%, 30%, 40%, 50%, 60%, 70%, 80%, 90%, or more, of the time during which transfection cocktail is added. In some embodiments, such mixing can be performed for at least or about 5 mins, 10 mins, 15 mins, 20 mins, 30 mins, 40 mins, 50 mins, 60 mins, 70 mins, 75 mins, mins, 90 mins, or 180 mins, or more, or a range including and between any two of the foregoing times, or some other range of time during which transfection cocktail is added.

Mixing during the period when transfection cocktail is being delivered or added to the sample of cells for transfection can be performed using any method or equipment known in the art. For example, in some embodiments, the cells can be suspended in culture medium in a stirred tank bioreactor which is actively stirred by an impeller. Mixing can be performed at any suitable rate and/or power input per unit volume of media (P/V) in the bioreactor which, in some embodiments, can be expressed as watts per cubic meter (W/m³). Thus, for example, in some embodiments, mixing during the period when transfection cocktail is being delivered or added to the sample of cells for transfection can be performed such that the power input per volume is at least or about 5, 10, 15, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 W/m³, or more, or some other value between, or range comprising, any of the foregoing specifically enumerated P/V values. Mixing can be performed at the same or different rate compared to mixing that may be used to grow or maintain the cells in suspension culture.

Optional Steps after the Addition of Transfection Cocktail to Cells

Once the transfection cocktail has been added or delivered to the sample of host cells, additional method steps can be performed, including for example, incubating cells to permit transfection to occur, stopping further transfection, incubating cells to permit biosynthesis of biological products directed by the genetic information embodied in the transfected nucleic acid, and downstream processing steps for purifying such biological products.

In some embodiments of the methods and systems of the disclosure, after all transfection cocktail has been added to the sample of cells for transfection, the mixture of cells and transfection cocktail can be incubated for some period to permit the cells to take up complexes of transfection reagent and nucleic acid (including, but not limited to PEI/pDNA complexes). In some embodiments, the transfection incubation time can be at least or about 0.5, 1, 1.5, 2, 2.5, 3, 3.5, 4, 4.5, 5, 5.5, 6, 7, 8, 9, or 10 hours, or more, some other value between, or range comprising, any of the foregoing specifically enumerated values of time.

In some embodiments, the mixture of cells and transfection cocktail can be stirred, agitated, or mixed during the transfection incubation period. During mixing, environmental factors, such as temperature, pH and oxygenation, can be controlled within acceptable ranges. In some embodiments, mixing can occur during the entire incubation period, or for a portion of such time, such as at least or about 10%, 20%, 30%, 40%, 50%, 60%, 70%, 80%, 90%, or more, of the incubation period. In some embodiments, such mixing can be performed for at least or about 1, 1.5, 2, 2.5, 3, 3.5, 4, 4.5, 5, 5.5, 6, 7, 8, 9, or 10 hours, or more, some other value between, or range comprising, any of the foregoing specifically enumerated values of time. Mixing during the transfection incubation period can be performed using any method or equipment known in the art. For example, in some embodiments, the cells can be suspended in culture medium in a stirred tank bioreactor which is actively stirred by an impeller. Mixing can be performed at any suitable rate and/or power input per unit volume of media. Thus, for example, in some embodiments, mixing during the incubation period can be performed such that the power input per volume is at least or about 5, 10, 15, 20, 21, 22, 23, 24, 25, 26, 27, 28, 29, 30, 31, 32, 33, 34, 36, 37, 38, 39, 40, 45, 50, 55, 60, 65, 70, 75, 80, 85, 90, 95, or 100 W/m³, or more, or some other value between, or range comprising, any of the foregoing specifically enumerated P/V values. Mixing can be performed at the same or different rate compared to mixing that may be used to grow or maintain the cells in suspension culture, or while adding transfection cocktail. In some embodiments, no active stirring is performed during the transfection incubation period.

In some embodiments of the methods and systems of the disclosure, quench media is added to the transfected cell sample to stop further uptake by cells of complexes of transfection reagent and nucleic acid (including, but not limited, to PEI/pDNA), thereby reducing cell toxicity. Quench media for use in the methods and systems of the disclosure can be added or delivered to a sample of transfected cells at any suitable percentage, on a weight by weight (w/w), weight by volume (w/v), or volume by volume (v/v) basis, of the volume or mass of the transfected cell sample (i.e., combined volume of cell sample and transfection cocktail). In some embodiments the percentage on a w/w, w/v, or v/v basis of quench media that is added to a transfected cell sample to stop transfection is at least or about 5, 6, 7, 8, 9, 10, 11, 12, 13, 14, 15, 16, 17, 18, 19, 25, 30, 35, or 40 percent of the volume or mass of the transfected cell sample, or more, or some other value between, or range comprising, any of the foregoing specifically enumerated values. In an exemplary non-limiting embodiment, transfection can be quenched by adding to a sample of transfected cells about 13% w/v CDM4 media, optionally including dextran sulfate.

In some embodiments of the methods and systems of the disclosure, transfected cells are incubated for time sufficient and under conditions suitable to permit expression of genetic information embodied in the nucleic acid transfected into the cells. In some embodiments, such expression will result in the biosynthesis of biological products, which may be released from and/or retained within the cells. In some embodiments, the post-transfection incubation period is at least or about 6, 7, 8, 9, 10, 11, 12, 15, 16, 18, 20, 24, 25, 30, 35, 36, 40, 42, 45, 48, 50, 54, 60, 65, 66, 68, 70, 72, 75, 80, 90, or 100 hours, or more, or some other time between, or range comprising, any of the foregoing specifically enumerated times.

In some embodiments, the transfected cells can be stirred, agitated, or mixed during the post-transfection incubation period. During mixing, environmental factors, such as temperature, pH and oxygenation, can be controlled within acceptable ranges. Media may be exchanged or added to the cell culture to maintain sufficiently high levels of nutrients and/or low levels of metabolic byproducts, such as by perfusion or supplemental feeding. During the post-transfection incubation period, samples of transfected cells or the media in which they are suspended may be taken and analyzed to detect expression of biological products. In some embodiments, mixing can occur during the entire incubation period, or for a portion of such time, such as at least or about 10%, 20%, 30%, 40%, 50%, 60%, 70%, 80%, 90%, or more, of the incubation period. In some embodiments, such mixing can be performed for at least or about 6, 7, 8, 9, 10, 11, 12, 15, 16, 18, 20, 24, 25, 30, 35, 36, 40, 42, 45, 48, 50, 54, 55, 60, 65, 66, 68, 70, 72, 75, 80, 90, or 100 hours, or more, or some other time between, or range comprising, any of the foregoing specifically enumerated times.

Mixing during the post-transfection incubation period can be performed using any method or equipment known in the art. For example, in some embodiments, the cells can be suspended in culture medium in a stirred tank bioreactor which is actively stirred by an impeller. Mixing can be performed at any suitable rate and/or power input per unit volume of media. Thus, for example, in some embodiments, mixing during the post-transfection incubation period can be performed such that the power input per volume is at least or about 5, 10, 15, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 95, or 100 W/m 3, or more, or some other value between, or range comprising, any of the foregoing specifically enumerated P/V values. Mixing can be performed at the same or different rate compared to mixing that may be used to grow or maintain the cells in suspension culture, while adding transfection cocktail to the cells, and/or during the transfection incubation period.

After the post-transfection incubation step, transfected cells and/or the media in which they were maintained after transfection can be processed further to isolate and purify biological products synthesized by the cells as a result of transfection. In some embodiments, where the biological product is secreted or otherwise released from intact cells, the media can be separated from the cells, such as by filtration, and then processed further to purify the product. In other embodiments, where biological product is retained within intact cells, the cells can be lysed to release the product into the surrounding media using any method known in the art, such as mechanically, for example, with a high pressure homogenizer or bead mill, or non-mechanically, which can encompass physical, chemical, or biological methods. Examples of physical methods include exposing cells to heating, freeze-thaw cycles, osmotic shock, sonication or cavitation; examples of chemical methods include treating cells with alkali or detergents; and examples of biological methods include treating cells with enzymes. After lysing cells, cellular debris and remnants can be removed in a variety of ways known in the art, such as centrifugation or filtration. Host cell DNA, such as genomic DNA, can be removed by treating the lysate with endonucleases such as Benzonase, or adding certain detergents to the lysate to precipitate the host cell DNA, forming a flocculant mass which can be separated from the supernatant. Partially clarified lysate, such as supernatant or filtrate, can then be subjected to additional downstream processing steps to purify the desired biological product.

Any suitable downstream processing steps are possible, given the nature of the biological product to be purified, for example precipitation in a lyotropic salt, such as ammonium sulfate, or chromatography. Many types of chromatography are known in the art including, without limitation, size exclusion chromatography (SEC); affinity chromatography (for example, in which an affinity ligand, such as an antibody, or antigen binding fragment thereof, lectin, protein A, protein G, protein L, or glycan, etc., capable of specifically binding to the biological product is attached to the stationary phase); immobilized metal chelate chromatography (IMAC); thiophilic adsorption chromatography; hydrophobic interaction chromatography (HIC); multimodal chromatography (MMC); pseudo-affinity chromatography; and ion exchange chromatography (IEX or IEC), such as anion exchange chromatography (AEX) or cation exchange chromatography (CEX). In other embodiments, the downstream processing step can comprise desalting or buffer exchange, filtering, such as ultrafiltration, nanofiltration, and/or diafiltration, or concentrating the biological product, for example using tangential flow filtration (TFF). Use of more than one downstream processing step is possible, and the plurality of downstream processing steps can be performed in any order according to the knowledge of those ordinarily skilled in the art.

In some embodiments, the biological product is a recombinant AAV vector, and the downstream step for purifying the vector is at least one chromatography step. In some embodiments, the chromatography step comprises antibody-based affinity ligand purification in which an antibody (e.g., an IgG), or antibody fragment thereof, or a single-chain camelid antibody (such as a heavy chain variable region camelid antibody), attached to a stationary phase specifically binds certain capsids. Non-limiting examples of affinity resins useful for purifying recombinant AAV vectors include Sepharose AVB, POROS CaptureSelect AAVX, POROS CaptureSelect AAV8, and POROS CaptureSelect AAV9. See, e.g., Terova, 0, et al., Affinity Chromatography Accelerates Viral Vector Purification for Gene Therapies, BioPharm Intl. eBook pp. 27-35 (2017); Mietzsch, M, et al., Characterization of AAV-Specific Affinity Ligands: Consequences for Vector Purification and Development Strategies, Mol. Ther. Meth. & Clin. Dev., 19:362-73 (2020); Rieser, R, et al., Comparison of Different Liquid Chromatography-Based Purification Strategies for Adeno-Associated Virus Vectors, Pharmaceutics 13, 748 (2021) (doi.org/10.3390/pharmaceutics13050748). In other embodiments, the ligand can be the same as or structurally related to a cell surface receptor molecule to which certain capsids specifically bind, such as a glycan, for example, sialic acid (e.g., an O-linked or N-linked sialic acid), galactose, heparin, or heparan sulfate, or a proteoglycan, such as a heparan or heparin sulfate proteoglycan (HSPG). For example, an affinity resin containing sialic acid residues can be used to purify recombinant AAV vectors comprising capsids that specifically bind to sialic acid (e.g., AAV1, AAV4, AAV5, or AAV6); an affinity matrix containing galactose can be used to purify vectors with capsids that specifically bind to galactose (e.g., AAV9); and an affinity matrix containing heparin, heparan, or HSPG can be used to purify AAV vectors with capsids that specifically bind to HSPG (e.g., AAV2, AAV3, AAV3b, AAV6, or AAV13). In yet other exemplary non-limiting embodiments, depending on the physicochemical characteristics of the vector, such as the charge on the capsid, AAV vectors can be further purified by anion exchange, cation exchange, or hydrophobic interaction chromatography, others being possible.

Before or during any stage of purification, the amount of a recombinant AAV vector in a sample can be quantified by a variety of techniques known in the art, such as by quantitative PCR (qPCR) using primers against the ITRs, or sequences in the transgene or other parts of the expression cassette, or using digital droplet PCR (ddPCR), and expressed as a titer in terms of vector genomes per unit volume, such as milliliters (vg/mL). See, e.g., Dobnik, D, et al., Accurate Quantification and Characterization of Adeno-Associated Viral Vectors, Front. Microbiol., Vol. 10, Art. 1570, pp. 1-13 (2019); Wang, Y, et al., A qPCR Method for AAV Genome Titer with ddPCR-Level of Accuracy and Precision, Mol. Ther.: Methods & Clin. Devel., 19:341-6 (2020); Werling, N J, et al., Systematic Comparison and Validation of Quantitative Real-Time PCR Methods for the Quantitation of Adeno-Associated Viral Product, Hum. Gene Ther. Meth. 26:82-92 (2015).

Before or during any stage of purification, the purity of a recombinant AAV vector in a sample can be determined and expressed in a variety of ways known in the art. For example, vector preparations can be analyzed on denaturing polyacrylamide gels and silver stained to detect proportions of the different viral proteins, VP1, VP2, and VP3, relative to cellular proteins. Different techniques can also be used to detect the proportion of full compared to empty capsids, with a greater percentage of full capsids indicating higher purity. As used herein, a “full capsid” is one that is concluded to contain a vector genome, and an “empty capsid” is a one that is concluded to contain either no or little nucleic acid. For example, capsids in vector preparations can be visualized using transmission electron microscopy, including cryoEM, and the numbers of full and empty capsids counted manually or using computerized image recognition algorithms. Even greater resolution can be achieved using analytical ultracentrifugation, which can discriminate between full, partially full and empty capsids.

A convenient method for estimating AAV vector purity in terms of amount of contaminating empty capsids is to measure the UV light absorbance of a vector preparation, such as a vector preparation purified by size exclusion chromatography, at 260 nm and 280 nm, and then calculating the absorption ratio at the two wavelengths (UV260/UV280 ratio). By calculating the theoretical extinction coefficients for a particular vector's capsid and genome, the relative concentrations of its capsid and genome in a preparation can be calculated from the UV260/UV280 ratio, with higher UV260/UV280 values indicating a greater proportion of full capsids.

Additional information about methods for testing vector purity are described in Burnham B, et al., Analytical ultracentrifugation as an approach to characterize recombinant adeno-associated viral vectors, Hum. Gene Ther. Meth., 26(6):228-242 (2015); Subramanian, S, et al., Filling Adeno-Associated Virus Capsids: Estimating Success by Cryo-Electron Microscopy, Hum. Gene Ther., 30(12):1449-60 (2019); McIntosh, N L, et al., Comprehensive characterization and quantification of adeno associated vectors by size exclusion chromatography and multi angle light scattering, Nat. Sci. Reports, 11:3012, pp. 1-12 (2021); Sommer, J M, et al., Quantification of Adeno-Associated Virus Particles and Empty Capsids by Optical Density Measurement, Mol. Ther., 7(1):122-8 (2003); Wu, D, et al., Rapid Characterization of AAV gene therapy vectors by Mass Photometry, bioRxiv 2021.02.18.431916 (doi.org/10.1101/2021.02.18.431916).

### Biological Products

The methods and systems for transfection of the disclosure can be used in the production of a variety of biological products that can be synthesized by transfected host cells. Biological products can be encoded by genetic information embodied in the transfected nucleic acid (for example, protein coding sequence in a DNA plasmid), but a biological product could also be produced by a cell using endogenous genetic information under the direction of exogenously introduced instructions. For example, a cell could be directed to produce a biological product it might not ordinarily produce but for the introduction via transfection of genetic information embodied in nucleic acid that activates transcription programs ordinarily quiescent, such as by transfection of plasmid DNA encoding a transcriptional activator or repressor protein. The construction of vectors, such as plasmids, suitable for expression of biological products after transfection into host cells is familiar to those of ordinary skill in the art. For example, a gene encoding a protein, or non-coding RNA molecule, can be cloned into an expression vector under the control of a constitutive or inducible transcription control element (e.g., promoter and enhancer), grown in bacteria to high levels, purified, and then used to transfect mammalian or other types of host cells in which the gene is expressed. See, e.g., Kaufman, R, Overview of Protein Expression in Mammalian Cells, Current Protocols in Molecular Biology, 14: 16.12.1-16.12.6 (1991); Hunter, M, et al., Optimization of Protein Expression in Mammalian Cells, Curr. Protoc. Protein Sci. 95(1):e77 (2019); Tripathi N K and Shrivastava A, Recent Developments in Bioprocessing of Recombinant Proteins: Expression Hosts and Process Development, Front. Bioeng. Biotechnol. 7:420 (2019).

Many examples of biological products will be familiar to those of ordinary skill in the art, and the type and nature of such products is not limiting. Examples include biological products that have therapeutic and/or prophylactic effects on diseases or disorders, including those of humans, animals or other organisms, as well as industrial applicability. Biological products can be secreted by transfected host cells into the media, or can be retained within the host cells, necessitating host cell disruption or lysis in order to liberate the products for subsequent purification. Biological products include, without limitation, peptides, polypeptides, or proteins of any kind, including glycoproteins or proteins having other types of post-translational modifications known in the art, such as covalent addition of lipid molecules. In some embodiments, proteins can include standard or non-standard amino acids, can have a wild type amino acid sequence, or be naturally occurring variants thereof, or be non-naturally variants or versions modified or engineered to possess novel properties, such as chimeric proteins, or fusion proteins, including fusions of a polypeptide or domain thereof with another polypeptide or domain thereof having a distinct function, such as protein fusions with the Fc region from an immunoglobulin (e.g., IgG) or albumin to extend the serum half-life of the fusion partner, such as an enzyme (e.g., a clotting factor). In other embodiments, proteins can be single chain polypeptides, or comprise multiple polypeptide chains, which may be covalently or non-covalently bound to each other. In some embodiments, proteins can be enzymes or zymogens with therapeutic or prophylactic utility (such as enzymes used in replacement therapy for any enzyme activity deficiency due to a deleterious mutations, such as mutations in genes encoding lysosomal enzymes, such as α-galactosidase, α-glucosidase, β-glucosidase, sphingomyelinase, galactocerebrosidase, or α-L-iduronidase), or industrial enzymes; clotting factors, such as Factor V, Factor Va, Factor VII, Factor VIIa, Factor VIII, Factor Villa, Factor IX, Factor IXa, Factor X, Factor Xa, or von Willebrand factor; antibodies, or antigen binding fragments thereof, of any type (e.g., IgG), clonality (e.g., monoclonal antibodies) or specificity; or growth factors, hormones, or cytokines, such as ILGF-1, ILGF-2, PDGF, EGF, NGF, NF-3, NF-4, BDNF, GDGF, Epo, TGF alpha, TGF beta, IFN alpha, IFN beta, IFN gamma, IL-2, IL-4, IL-12, GMCSF, lymphotoxin, insulin, glucagon, thyroid hormone, thyroid stimulating hormone, parathyroid hormone, or growth hormone). In some embodiments, biological products can be proteins or other molecules derived from microorganisms, such as parasites, fungi, bacteria, and viruses, or from cancer cells, or fragments, regions, or domains of such proteins or molecules, for use as antigens in vaccines, or components thereof. In other embodiments, biological products include lipids, carbohydrates, and nucleic acids.

In other embodiments, biological products can be large supramolecular complexes, such as subcellular organelles (e.g., ribosomes, mitochondria, etc.), vaccines, viruses (e.g., baculovirus, vaccinia virus, adenovirus, adeno-associated virus, lentivirus, herpes virus, etc.), modified viruses engineered to kill cancer cells (oncolytic viruses), or recombinant vectors, including for use in gene therapy, derived from viruses or that use viral components, non-limiting examples of which include recombinant adenoviral (AdV) vectors, adeno-associated viral (AAV) vectors (or derived from other types of parvovirus), or lentiviral vectors (e.g., derived from HIV or other retroviruses).

### Adeno-Associated Viral (AAV) Vectors

The methods and systems for transfection of the disclosure can be used to produce, in transfected host cells, recombinant vectors derived from adeno-associated virus (AAV), i.e., adeno-associated viral (AAV) vectors, which can be used for gene therapy to prevent or treat disorders and diseases of animals, including those of humans. Such AAV vectors can include numerous types of capsids and transgenes as are known in the art or are yet to be developed.

As is well known in the art, AAV is a small non-enveloped, apparently non-pathogenic virus that depends on certain other viruses to supply gene products, known as helper factors, essential to its own replication, a quirk of biology that has made AAV well-suited to serve as a recombinant vector. For example, adenovirus (AdV) can serve as a helper virus by providing certain adenoviral factors, such as the E1A, E1B55K, E2A, and E4orf6 proteins, and the VA RNA, in cells co-infected by adenovirus and AAV. Numerous types of AAV have been discovered which are restricted in their ability to infect certain animals (such as mammal and bird) and species (such as human and rhesus monkey), and having a tendency within species to infect certain tissues (such as liver or muscle) more so than others, a phenomenon called tissue tropism, based on specific binding to different cell surface receptors. One type of AAV that infects humans, called AAV2, is particularly well characterized biologically, although many other types have found utility in creating gene therapy vectors.

In nature, the AAV genome is a single strand of DNA, about 4.7 kilobases long in AAV2, which contains two genes called rep and cap. By virtue of alternative splicing of the transcripts from two promoters, the rep gene produces four related multifunctional proteins called Rep (Rep78, Rep68, Rep52 and Rep40 in AAV2) which are involved in replication and packaging of the genome, and expression of the viral genes. Alternative splicing of the transcript from the single promoter controlling the cap gene produces three related structural proteins, VP1, VP2, and VP3, a total of 60 of which self-assemble to form the virus's icosahedral capsid in a ratio of approximately 1:1:10, respectively. VP1 is longest of the three VP proteins, and contains amino acids in its amino terminal region not present in VP2, which in turn is longer than VP3 and contains amino acids in its amino terminal region not present in VP3. The capsid encloses and protects the AAV genome, and also is responsible for specific binding to cell surface receptors and intracellular trafficking to the nucleus.

In addition to the rep and cap genes, intact AAV genomes have a relatively short (145 nucleotides in AAV2) sequence element positioned at each of their 5′ and 3′ ends called an inverted terminal repeat (ITR). ITRs contain nested palindromic sequences that can self-anneal through Watson-Crick base pairing to form a T-shaped, or hairpin, secondary structure. In AAV2, ITRs have important functions required for the viral life cycle, including converting the single stranded DNA genome into double stranded form required for gene expression, as well as packaging by Rep proteins of single stranded AAV genomes into capsid assemblies.

After an AAV2 virion binds its cognate receptor on a cell surface, the viral particle enters the cell via endocytosis. Upon reaching the low pH of lysosomes, capsid proteins undergo a conformational change which allows the capsid to escape into the cytosol and then be transported into the nucleus. Once there, the capsid disassembles, releasing the genome which can be acted on by cellular DNA polymerases to synthesize the second DNA strand starting at the ITR at the 3′ end, which functions as a primer after self-annealing. Expression of the rep and cap genes into mRNA and proteins can then commence, followed by formation of new viral particles.

The relative simplicity of AAV structure and life cycle, and the fact that it is not known to be pathogenic in humans, inspired investigators to engineer AAV and adapt it to serve as a recombinant vector for gene therapy. As originally conceived, this was done by cloning the entire genome of AAV2, including both ITRs, into a plasmid, removing the rep and cap genes into a separate plasmid, and replacing them with a gene expression cassette comprising a heterologous transcription control region operably linked with a transgene encoding an antibiotic resistance marker. In the second plasmid, the AAV2 genome including the rep and cap genes, but lacking the ITRs, was instead flanked by adenovirus terminal repeats which could enhance expression of the rep and cap genes, but would neither homologously recombine with the AAV ITRs nor support packaging of the rep and cap genes into capsids. These two plasmids, the genome plasmid and rep/cap helper plasmid, were then transfected into mammalian cells which had been infected with adenovirus to provide helper factors. Recombinant AAV virions were produced which could transduce host cells and confer resistance to the antibiotic. Samulski, R J, et al., Helper-Free Stocks of Recombinant Adeno-Associated Viruses: Normal Integration Does Not Require Viral Gene Expression, J. Virol. 63(9):3822-8 (1989); Xiao, X, et al., Adeno-associated virus (AAV) vectors for gene transfer, Adv. Drug Deliv. Revs. 12:201-15 (1993).

Co-infection with a helper virus was considered undesirable, however, because of the helper viruses, mainly adenovirus and herpes simplex virus, are both known human pathogens. Later research clarifying which viral helper factors were essential for AAV replication allowed researchers to express these factors from genes provided on a separate plasmid transfected into cells and found it was possible to efficiently produce recombinant AAV vectors without relying on helper virus co-infection. Evidently, Rep, the capsid proteins (VP1, VP2, VP3), and the AdV helper factors were expressed and functioned in the cells to assemble and package capsids with vector genomes copied from the plasmid containing its sequence. Experimenting with different arrangements of elements, the researchers successfully produced high levels of recombinant AAV vectors when genes for the adenovirus helper factors contained in one plasmid, the AAV rep and cap genes contained in a second plasmid, and the vector genome contained in a third plasmid were transfected into cells (so-called triple transfection technique), as well as when the rep and cap genes, and vector genome, were combined in a single plasmid (allowing for transfection with just two plasmids). Grimm, D, et al., Novel tools for production of recombinant adenoassociated virus vectors, Hum Gene Ther 9:2745-60 (1998); Matsushita, T, et al., Adeno-associated virus vectors can be efficiently produced without helper virus, Gene Ther. 5:938-45 (1998); Xiao, X, et al., Production of high-titer recombinant adeno-associated virus vectors in the absence of helper adenovirus, J. Virol. 72:2224-32 (1998).

In the approach for producing vectors outlined above, the only viral sequences retained in the vector genome are the ITRs, which are required for their essential role in packaging the genome into capsids and expressing the transgene after transducing target cells. Because the rep and cap genes exist outside their usual context flanked by ITRs, they are not packaged into the vectors. Consequently, while vectors, like viruses, are able to bind to target cells and convey their genomes into the cells, they cannot replicate and create new vector particles. For this reason, the term “transduction” is often used to refer to this process in place of the term “infection.”

Although alternative approaches have been developed for producing recombinant AAV vectors, such as use of the baculovirus system in insect cells, transfection of host cells with expression vectors comprising the genetic information required for AAV vector biosynthesis remains an effective method. Accordingly, the transfection methods and systems of the disclosure can usefully be applied to producing AAV vectors of any design in host cells, particularly at larger scales, where previous transfection methods may be less efficient. In some embodiments, the methods and systems of the disclosure can be used to transfect host cells with expression vectors, such as plasmids, comprising an AAV rep gene, an AAV cap gene, an AAV vector genome comprising a gene of interest, and genes for viral helper factors. The aforementioned genetic information can be included in any number of plasmids, such as a single plasmid containing all the genes required for AAV vector production, or a plurality of plasmids in which the genes can be included in different combinations and arrangements. In some embodiments, a separate plasmid can be used to contain each of the genes required for AAV vector production.

Any plasmid known in the art to be suitable for expressing exogenous genes after transfection into host cells, such as mammalian host cells, such as HEK293, HeLa, A549, BHK, Vero, or other mammalian cells or cell lines, can be used. As known in the art, plasmids can contain a backbone originating with the plasmid as it occurred in nature, which can be modified, such as by deleting unnecessary sequences and adding exogenous sequences that confer some desired property. For example, plasmids often contain a bacterial origin of replication (ORI) and a bacterial antibiotic resistance gene (e.g., for ampicillin, kanamycin, etc.), which allows plasmids to be grown to very high copy number in bacteria (e.g., E. coli, etc.) after which they can be purified and used to transfect eukaryotic host cells. Exemplary non-limiting plasmid backbones include pUC, pBR322, pSC101, pGEM, with many others known in the art. Plasmids can also usefully contain a cloning site, or multiple cloning site (MCS), which provides convenient restriction enzyme sites for insertion of exogenous DNA sequences into the plasmid. In other embodiments, plasmids can further include a promoter to drive expression of a gene inserted in the MCS, a transcription terminator element (e.g., a polyA signal sequence) to end transcription of a gene inserted in the MCS. In some embodiments, plasmids can contain viral origins of replication, such as the Epstein-Barr virus (EBV) or SV40 virus ORI, which allows episomal amplification of plasmids after transfection into mammalian cells expressing the EBV EBNA1 or SV40 large T antigen proteins, respectively. Numerous other elements can be included in plasmids, and plasmids useful for expressing genes in mammalian and other types of cells can be constructed using different methods known in the art. See, e.g., Gill, D R, et al., Progress and Prospects: The design and production of plasmid vectors, Gene Ther., 16:165-71 (2009); Plasmids 101: A Desktop Reference (3^{rd} Ed.), Addgene (2017).

Although use of plasmids is often convenient to introduce the genetic elements required for recombinant AAV vector production into host cells by transfection, other types of DNA expression vectors can be used as well, non-limiting examples being minicircle DNA and covalently closed linear DNA construct known as Doggybone DNA. See, e.g., Gill, D R, et al., Progress and Prospects: The design and production of plasmid vectors, Gene Ther., 16:165-71 (2009); Scott, V L, et al., Novel synthetic plasmid and Doggybone™ DNA vaccines induce neutralizing antibodies and provide protection from lethal influenza challenge in mice, Human Vaccines & Immunotherapeutics, 11(8):1972-82 (205), DOI: 10.1080/21645515.2015.1022008.

In some embodiments, a triple plasmid format such as that described above can be used in connection with the methods and systems for transfection of the disclosure. In such embodiments, a first plasmid can contain the genome of an AAV serotype or variant, such as AAV2, or others, including the rep and cap genes (rep/cap plasmid) and excluding the viral ITR sequences; a second plasmid can contain the vector genome sequence flanked at the 5′ and 3′ ends by an AAV ITR (vector plasmid); and a third plasmid can contain the genes for expressing the viral helper factors (helper plasmid). In the rep/cap plasmid, the AAV genome can be included without modification except for deletion of the ITR sequences. In this embodiment, the rep and cap genes can be expressed from their native promoters. In other embodiments, however, particularly when it is desired to express rep and cap genes from different AAV viruses (e.g., Rep from AAV2 and cap gene from a different serotype or variant), the coding sequences for the rep and cap genes can be included in a plasmid as separate transcriptional units controlled by the native promoters or by heterologous promoters. For example, the rep gene could be included in the rep/cap plasmid controlled by its native promoters (p5 and p19 in the case of AAV2), whereas the cap gene could be controlled by a promoter constitutively active in the host cells instead of its native promoter. The different transcription units could be inserted into the rep/cap plasmid so that they are transcribed in the same direction or in different directions. Promoter sequences, translation initiation sites, and RNA splice sites that they exist in the native AAV genomic sequences can be modified any way known in the art to modulate the proportions of the different Rep and Cap proteins expressed from the rep/cap plasmid. As noted, the rep and cap genes can originate from the same type of AAV, such as AAV2 with others possible, or the rep and cap genes can originate from different types of AAV. In some embodiments, the rep gene from AAV2 is used and the cap gene is chosen from a type of AAV other than AAV2. As with the rep and cap genes, the sequences for expressing the viral helper factors can be included in the helper plasmid as they exist in the genome of the virus from which they are derived, or they can instead be included as separate transcriptional units controlled by native or heterologous promoters, and be inserted into the helper plasmid in any suitable arrangement or direction, or can be included as separate transcriptional units on separate plasmids.

Although the triple transfection approach is frequently used, it is not the only approach possible, and in other embodiments, the elements required for producing recombinant AAV vectors can be included on fewer or more plasmids. For example, in some embodiments, the AAV rep and cap genes, and sequences for expressing viral helper factors can all be included on one plasmid, whereas the vector genome is provided on a second plasmid. In another embodiments of the two plasmid approach, the AAV rep and cap genes, and sequence of the vector genome can be included on one plasmid, and the sequences for expressing the viral helper factors can be included on the second plasmid. In yet another approach, four plasmids can be used, one containing the sequence of the vector genome, a second containing the sequences for expressing viral helper factors, a third containing the AAV rep gene, and a fourth containing the AAV cap gene controlled by a heterologous promoter. Other configurations and arrangements are also possible, as will be appreciated by those of ordinary skill in the art. The different plasmids, in some embodiments, can be replicated to high copy numbers in different bacterial cultures, purified, and then combined in any desired stoichiometric ratios to transfect host cells and produce AAV vectors.

Any viral helper factors known in the art to be effective to produce recombinant AAV vectors can be used in connection with the methods and systems of the disclosure. In some embodiments, the helper virus is HSV-1, and exemplary helper factors include the HSV-1 gene products UL5, UL8, UL52, and ICP8. In other embodiments, the helper virus is adenovirus 5, and exemplary helper factors include the AdV5 gene products E1A, E1B55K, E2A, E4orf6, and VA RNA.

In other embodiments, the helper virus is HPV-16, and exemplary helper factors include the HSV-16 gene products E1, E2, and E6. And in yet other embodiments, the helper virus is HBoV1, and exemplary helper factors include the HBoV1 gene products NS2, NS4, NP1, and BocaSR. More information about such helper factors can be found in, e.g., Meier, A F, et al., The Interplay between Adeno-Associated Virus and Its Helper Viruses, Viruses 12:662 (2020), doi:10.3390/v12060662. In some embodiments, production of recombinant AAV vectors can be performed using host cells that constitutively express one or more viral helper factors, in which case it may not be necessary to provide all essential helper factors via transfection. Thus, for example, it is known that HEK293 cells constitutively express adenovirus helper factors E1A and E1B, such that helper plasmid or plasmids need only contain sequences for expressing the essential viral helper factors E2A, E4orf6, and VA RNA. While it will often be desirable to express viral helper factors from plasmids or other expression vectors transfected into host cells, production of recombinant AAV vectors using co-infection with a helper virus, such as AdV5 or others is not foreclosed in connection with use of the methods and systems of the disclosure.

In some embodiments, the methods and systems of the disclosure can be used in connection with cell lines that stably express some of the elements required to produce recombinant AAV vectors that would otherwise need to be provided via transfection. For example, packaging cell lines contain stably integrated AAV rep and cap genes, and production of vectors in such cells requires them to be transiently transfected with a plasmid containing an AAV vector genome, as well as infection with a helper virus. Packaging cells are described further in, e.g., Clement, N and J C Grieger, Manufacturing of recombinant adeno-associated viral vectors for clinical trials, Mol. Ther. Meth. & Clin. Dev. (2016) 3, 16002 (doi:10.1038/mtm.2016.2).

Recombinant AAV vectors produced in connection with use of the methods and systems for transfection of the disclosure can include any gene of interest within an AAV vector genome of any sequence, structure, arrangement of functional sub-elements, and configuration suitable for its intended use, such as use in gene therapy. As AAV vectors are typically designed, choice of the gene of interest is limited only by the packaging capacity of the capsid, so that the gene's length, when combined with all other elements in the genome required for vector function, such as the transcription control region and the ITRs, does not exceed approximately 5 kilobases in the case of AAV2, although experimental strategies have been developed to surpass this packaging limit.

For purposes of gene therapy, a gene of interest can be any gene, the product of which would be understood to prevent or treat, but not necessarily cure, any disease or condition. In some embodiments, gene therapy is intended to prevent or treat a disease or condition characterized by an abnormally low amount or even absence of a product produced by a naturally occurring gene, such as might occur due to a loss of function mutation. Relating to such embodiments, the gene of interest can be one intended to compensate for the defective gene by providing the same or similar gene product when expressed. A non-limiting example would be a vector designed to express a functional version of clotting factor IX for use in gene therapy of hemophilia B, which is caused by a loss of function mutation in the native factor IX gene. In other embodiments, however, the gene of interest could be one intended to counteract the effects of a deleterious gain of function mutation in targeted cells. In some embodiments, the gene of interest can encode a transcriptional activator to increase the activity of an endogenous gene which produces a desirable gene product, or conversely a transcriptional repressor to decrease the activity of an endogenous gene which produces an undesirable gene product. In some embodiments, the gene of interest can encode for a protein (though messenger RNA) (including such proteins described in the prior section as examples of biological products that may be produced by transfected cells), or an RNA molecule with a function distinct from encoding protein, such as antisense RNA, or a regulatory non-coding RNA molecule, such as micro RNA (miRNA), short interfering RNA (siRNA), short hairpin RNA (shRNA), piwi-acting RNA, enhancer RNA, or long non-coding RNA. Protein coding sequences in a gene of interest can be codon-optimized, and translation start sites (e.g., Kozak sequence or non-consensus start sites) can be modified to increase or decrease their tendency to initiate translation. In some embodiments, the gene of interest can encode more than one open reading frame (and thus produce polypeptides with distinct sequences) by virtue of using alternative promoters, alternative translation start sites, and/or alternative splice sites. In other embodiments, a vector genome can comprise more than one gene of interest, each part of its own separate transcriptional unit. In some embodiments, the product of the gene of interest remains inside the cell in which it is expressed, and/or is secreted from cells in which expressed to act elsewhere in an organism.

Apart from the gene of interest, the transcription control region, which is operably linked with and controls the transcription of the gene of interest in transduced target cells, is amenable to design choice and optimization depending on the intended use of the vector. In some embodiments, transcription control regions comprise a promoter for recruiting the RNA polymerase transcription complex, as well as optionally one or more enhancer elements which can function to increase the rate of transcription.

Transcriptional control regions can be constitutively active, meaning they are capable of expressing transgenes in many different cell types. Examples include control regions from certain viruses, such as the CMV IE promoter/enhancer, RSV promoter/enhancer, or SV40 promoter, or from house-keeping genes that are active in most eukaryotic cells, such as dihydrofolate reductase gene promoter, cytoplasmic β-actin gene promoter, or the phosphoglycerol kinase (PGK) gene promoter, many others being known. In other embodiments, transcriptional control regions can be tissue specific, meaning that they are only, mostly or at least preferentially active in specific types of cells, such as liver, muscle, or neuronal cells. In yet other embodiments, transcriptional control regions can be inducible, meaning that they are inactive, or only minimally active, in the absence of certain environmental conditions, such as elevated temperature or hypoxia, or unless certain chemicals or compounds are present, such as drugs (e.g., antibiotics) or toxins (e.g., heavy metals).

A transcription control region can comprise the same nucleotide sequence as would occur in a gene naturally, or be modified to improve its function and/or reduce its length by changing, adding or removing nucleotides relative to a sequence found in nature, or even be entirely synthetic. Transcription control regions can be derived from the same gene as is the transgene (homologous). Alternatively, a transcription control region can be derived from an entirely different gene than the gene from which the transgene is derived (heterologous). Transcription control regions can be hybrid by including a promoter from one type of gene and combining it with one or more enhancers from one or more different genes, including genes from different species. As arranged in a vector genome, enhancer elements may be contiguous with or adjacent to the promoter, or can instead be positioned at some distance upstream or downstream of the promoter. In some embodiments, an enhancer element that would ordinarily be present as a single copy in its native context can be provided in multiple copies.

Apart from the gene of interest and transcription control region, many other aspects of AAV vector genomes are amenable to design choice and optimization depending on the intended use of the vector. In some embodiments, vector genomes can further comprise untranslated regions from the 5′ and/or 3′ end of genes, additional stop codons, non-coding exons, introns, stuffer and filler sequences, transcriptional termination signals (e.g., polyA signal sequence), elements that stabilize RNA transcripts, splice donor and acceptor sites, lox sites, binding sites for regulatory miRNAs, elements that enhance nuclear export of mRNAs (such as the woodchuck hepatitis virus post-transcriptional regulatory element (WPRE)), and any other element demonstrated empirically to improve expression of a gene of interest, even if the mechanism may be uncertain.

In some embodiments, a vector genome can be designed for purposes of editing or otherwise modifying the genome of a target cell. For example, a vector genome can include a gene of interest flanked by homology arms intended to promote homologous recombination between the vector genome and the target cell genome. In another example, a vector genome can be designed to carry out CRISPR gene editing by expressing a guide RNA (gRNA) and/or an endonuclease, such as Cas9 or related endonucleases, such as SaCas9, capable of binding the gRNA and cleaving a DNA sequence targeted by the gRNA. Other strategies for genome editing known in the art may also be implemented via AAV vectors, such as expression of engineered zinc finger nucleases.

As known in the art, the ITRs typically used in AAV vectors originate from AAV2, but ITRs derived from other serotypes and naturally occurring AAV isolates, or hybrid, or even entirely synthetic ITRs, may be used as well. In some non-limiting embodiments, vector genomes include two intact ITRs, one at each end of the single stranded DNA genome. In other embodiments, however, AAV vectors can be produced so that a mutated third ITR lacking a terminal resolution site is positioned at or near the center of the genome. These so-called self-complementary AAV (scAAV) genomes can self-anneal into double stranded form after capsid uncoating, permitting gene expression to proceed immediately without need for second strand synthesis, as is the case with conventional single stranded AAV genomes. ITRs originating from one type of AAV may be used in vectors in which the capsid originates from the same type of AAV, or a different type of AAV (which are known as pseudotyped vectors). For example, AAV2 ITRs may be used in a genome encapsidated by an AAV2 capsid, or an AAV5 capsid (a pseudotyped vector which is denoted AAV2/5) or some other capsid from an AAV other than AAV2.

Just as there is wide latitude in the design of vector genomes, AAV vectors can be made using many different naturally occurring and modified AAV capsids. At one time, only six types of primate AAV had been isolated from biological samples (AAV1, AAV2, AAV3, AAV4, AAV5, and AAV6), the first five of which were sufficiently distinct structurally to be classified as different serotypes based on antibody cross reactivity experiments. Later, two novel AAVs, called AAV7 and AAV8 were discovered by PCR amplification of DNA from rhesus monkeys using primers targeting highly conserved regions in the cap genes of the previously discovered AAVs. Gao, G, et al., Novel adeno-associated viruses from rhesus monkeys as vectors for human gene therapy, PNAS (USA) 99(18):11854-11859 (2002). Subsequently, a similar approach was used to clone numerous novel AAVs from human and non-human primate tissues, vastly expanding the scope of known AAV cap protein sequences. Gao, G, et al., Clades of Adeno-Associated Viruses Are Widely Disseminated in Human Tissues, J Virol. 78(12):6381-6388 (2004). Many AAV cap protein sequences are highly similar to each other, or previously identified AAVs, and while often referred to as distinct AAV “serotypes,” not all such capsids would necessarily be expected to be immunologically distinguishable if tested by antibody cross reactivity.

Research has established that different AAV capsids have different tissue tropisms, as well as other properties that may make one capsid preferable over another for particular applications. For example, depending on which population is being tested, humans may have high neutralizing antibody titers as a result of exposure to naturally occurring AAVs, which can interfere with the ability of AAV vectors with the same or similar capsids to transduce target cells. Thus, in designing a vector for gene therapy, choice of capsid may in some cases be guided by the immunogenicity of the capsid, and/or the seroprevalence of the patients to be treated.

AAV vectors which can be produced from cells transfected using the methods and systems of the disclosure can include any capsid known in the art to be suitable for its intended use, such as use in gene therapy. Such capsids include those from naturally occurring AAVs, as well as modified or engineered capsids. For example, naturally occurring capsids can be modified by inserting peptides, or making amino acid substitutions, in the cap protein sequence intended to improve capsid function in some way, such as tissue tropism, immunogenicity, stability, or manufacturability. Other examples include novel capsids with improved properties created by swapping amino acids or domains from one known capsid to another (which are sometimes known as mosaic or chimeric capsids), or which are generated and selected employing DNA shuffling and directed evolution methods. In some exemplary, non-limiting, embodiments, AAV vectors produced by transfected host cells can include any of the following capsids: AAV1, AAV2, AAV3, AAB3A, AAV3B, AAV4, AAV5, AAV6, AAV7, AAV8, AAV9, AAV10, AAV11, AAV12, AAV-Rh10, AAV-Rh74, AAV-DJ, AAV-DJ/8, AAV-DJ/9, AAV-LK03, AAV-PHP.B, AAV-Anc80, AAV2.5, AAV2i8, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, AAVHSC15, AAVHSC16, AAVHSC17, RHM4-1, RHM15-1, RHM15-2, RHM15-3/RHM15-5, RHM15-4, RHM15-6, AAV-NP22, AAV-NP66, AAV9.24, AAV9.45, AAV9.61, AAV8G9, AAV-TT, or AAVhu.37, with many others being possible. See, e.g., and without limitation, AAV capsid proteins described in WO 2015/121501 and WO 2017/023724).

In some embodiments, use of the methods and systems for transfection of the disclosure is effective to produce recombinant AAV vectors at high titers and purity. In some embodiments, a purified preparation of recombinant AAV vector produced by transfection using the methods and systems of the disclosure can calculated to have a titer of at least or about 1×10⁹, 1×10¹⁰, 1×10¹¹, 1.5×10¹¹, 2×10¹¹, 2.5×10¹¹, 3×10¹¹, 3.5×10¹¹, 4×10¹¹, 4.5×10¹¹, 5×10¹¹, 5.5×10¹¹, 6×10¹¹, 6.5×10¹¹, 7×10¹¹, 7.5×10¹¹, 8×10¹¹, 8.5×10¹¹, 9×10¹¹, 9.5×10¹¹, 1×10¹², 1.25×10¹², 1.5×10¹², 1.75×10¹², 2×10¹², 2.25×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², or 1×10¹³ vector genomes per milliliter (vg/mL) of cell suspension after transfection, or more, or a titer between, or range comprising, any of the foregoing specifically enumerated values. In some embodiments, a purified preparation of recombinant AAV vector produced by transfection using the methods and systems of the disclosure can have an A260/A280 ratio of at least or about 0.4, 0.5, 0.6, 0.7, 0.8, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, or 1.80, or more, or an A260/A280 ratio between, or range comprising any of the foregoing specifically enumerated values. In other embodiments, a purified preparation of recombinant AAV vector produced by transfection using the methods and systems of the disclosure can have purity expressed as the percentage of full capsids in a vector preparation which can be at least or about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or more, or any percentage of full capsids between, or range comprising any of the foregoing specifically enumerated values.

In some embodiments of the methods and systems of the disclosure for transfection in which three plasmids are used to produce recombinant AAV vectors, the three types of plasmid can be used in transfection in equal molar ratios, or unequal molar ratios. Thus, for example, in some non-limiting embodiments, the molar ratios of the first, second and third types of plasmid in the nucleic acid solution or transfection cocktail can be 1:1:1, 1:1:2, 1:1:3, 1:2:1, 1:2:2, 1:2:3, 1:3:1, 1:3:2, 1:3:3, 2:1:1, 2:1:2, 2:1:3, 2:2:1, 2:2:2, 2:2:3, 2:3:1, 2:3:2, 2:3:3, 3:1:1, 3:1:2, 3:1:3, 3:2:1, 3:2:2, 3:2:3, 3:3:1, 3:3:2, 3:3:3, 1:2:2, 1:2:3, or 1:3:3, with a deviation of the first, second or third values not exceeding±30%, ±20%, ±10%, or ±5%. In some embodiments, the first type of plasmid comprises AAV rep and cap genes, the second type of plasmid comprises sequences for expressing viral helper factors, and the third type of plasmid comprises the sequence of an AAV vector genome. In any of these embodiments, the host cells can be HEK293 cells, or derivatives thereof, or other cells, and the AAV vector can comprise an AAV9 capsid, or another capsid.

In some embodiments, methods and systems of the disclosure for continuous transfection of host cells can be used or configured to efficiently produce recombinant AAV vectors at large scale. Thus, for example, in some embodiments, volumes of host cells (such as HEK293 cells, and derivatives thereof) in culture (before transfection) of at least or about 100, 200, 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 6000, 7000, 8000, 9000, or 10000 L, or more, or some other value between, or range comprising, any of the foregoing specifically enumerated values can be transfected to produce recombinant AAV vectors. In any of these embodiments, the host cells can be HEK293 cells, or derivatives thereof, or other cells, and the AAV vector can comprise an AAV9 capsid, or another capsid.

In some embodiments, the methods and systems of the disclosure for continuous transfection of cells can be used or configured to efficiently produce AAV vectors at large scale (for example, in cell culture volumes of at least or about 100 L, 500 L, 1000 L, 2000 L, 5000 L, or more before transfection) by transfecting host cells with transfection cocktail that had been incubated for less than or about 25, 20, 15, 10, 9, 8, 7, 6, 5, 4, 3 minutes or less time, such as less than our about 175, 170, 165, 160, 155, 150, 145, 140, 135, 130, 125, 120, 115, 110, 105, 100, 90, 85, 80, 75, 70, 65, 60, 55, 50, 45, 40, 35, or 30 seconds, or less time, or some other value between, or range comprising, any of the foregoing specifically enumerated values. For example, in some embodiments, the incubation time can be about 30 to 180 seconds, 30 to 150 seconds, to 135 seconds, 45 to 135 seconds, 60 to 135 seconds, or 90 to 135 seconds, such as about 135 seconds. In any of these embodiments, the host cells can be HEK293 cells, or derivatives thereof, or other cells, and the AAV vector can comprise an AAV9 capsid, or another capsid.

In some embodiments, the methods and systems of the disclosure for continuous transfection of cells can be used or configured to efficiently produce AAV vectors at large scale (for example, in cell culture volumes of at least or about 100 L, 500 L, 1000 L, 2000 L, 5000 L, or more before transfection) by transfecting host cells with a predetermined volume of transfection cocktail, such as substantially the entire volume of transfection cocktail, which is added to the cells in culture in less than or about 90, 80, 70, 60, 50, 45, 40, 35, 30, 25, 20, 15, or 10 minutes, or less time, or some other value between, or range comprising, any of the foregoing specifically enumerated values. In some embodiments the addition time can be about 10 to 60 minutes, 10 to 30 minutes, 15 to 60 minutes, 15 to 30 minutes, or 30 to 60 minutes. In any of these embodiments, the host cells can be HEK293 cells, or derivatives thereof, or other cells, and the AAV vector can comprise an AAV9 capsid, or another capsid.

In some embodiments, the methods and systems of the disclosure for continuous transfection of cells can be configured so that AAV vectors can be produced at large scale (for example, in cell culture volumes of at least or about 100 L, 500 L, 1000 L, 2000 L, 5000 L, or more before transfection) while the flow of transfection cocktail within the system does not exceed a Reynold's number (Re) value of 5500, 5000, 4500, 4000, 3500, 3400, 3300, 3200, 3100, 3000, 2900, 2800, 2700, 2600, 2500, 2400, 2300, 2200, 2000, 1000, or 500, or less, or some other value between, or range comprising, any of the foregoing specifically enumerated values. In some embodiments, the methods and systems of the disclosure for continuous transfection of cells can be used or configured so that AAV vectors can be produced in a cell culture volume of at least 1000 L while the flow of transfection cocktail within the system does not exceed a Reynold's number (Re) value of 3500 or 4000. In any of these embodiments, the host cells can be HEK293 cells, or derivatives thereof, or other cells, and the AAV vector can comprise an AAV9 capsid, or another capsid.

In some embodiments, the methods and systems of the disclosure for continuous transfection of cells can be used or configured to efficiently produce AAV vectors at large scale (for example, in cell culture volumes of at least or about 100 L, 500 L, 1000 L, 2000 L, 5000 L, or more before transfection) by transfecting host cells with transfection cocktail comprising PEI and plasmid DNA. In some of these embodiments, sufficient pDNA is used to prepare transfection cocktail such that cells are transfected with at least or about 0.1, 0.2, 0.25, 0.3, 0.4, 0.5, 0.6, 0.65, 0.75, 0.8, 0.85, 0.9, 1, 2, 3, 4, 5, 6, 7, 8, 9, or 10 μg per 1×10⁶ viable cells, or more, or some other value between, or range comprising, any of the foregoing specifically enumerated values, such as about 0.1 to 10 μg per 1×10⁶ viable cells, 0.25 to 1.5 μg pDNA per 10⁶ viable cells, 0.25 to 7.5 μg per 1×10⁶ viable cells, 0.5 to 5 μg per 1×10⁶ viable cells, 0.5 to 2.5 μg per 1×10⁶ viable cells, to 1.0 μg pDNA per 10⁶ viable cells, 0.5 to 0.75 μg pDNA per 10⁶ viable cells, such as greater than 0.25 μg pDNA per 10⁶ viable cells, or about 0.5 μg pDNA per 10⁶ viable cells, or about 0.75 μg pDNA per 10⁶ viable cells. In some of these embodiments, sufficient PEI is used to prepare transfection cocktail such that the mass ratio of PEI to pDNA is at least or about 0.1, 0.5, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4, 5, 6, 7, 8, 9, or 10, or some other value between, or range comprising, any of the foregoing specifically enumerated values, for example, about 1.4 to 3.0, 1.8 to 2.6, 2.0 to 2.4, or about 2.2. In some of these embodiments, transfection cocktail is prepared containing sufficient pDNA such that cells are transfected with about 0.75 μg pDNA per 10⁶ viable cells and sufficient PEI such that the mass ratio of PEI to pDNA is about 2.2. In any of these embodiments, PEI can be linear PEI, such as linear fully depropionylated PEI, such as 40 kDa linear fully depropionylated PEI. In any of these embodiments, the host cells can be HEK293 cells, or derivatives thereof, or other cells, and the AAV vector can comprise an AAV9 capsid, or another capsid.

In some embodiments, the methods and systems of the disclosure for continuous transfection of cells can be used or configured to efficiently produce AAV vectors at large scale (for example, in cell culture volumes of at least or about 100 L, 500 L, 1000 L, 2000 L, 5000 L, or more before transfection) by transfecting host cells with transfection cocktail prepared from a transfection reagent solution comprising PEI and a nucleic acid solution comprising plasmid DNA, in which the PEI concentration (w/v) in the transfection reagent solution ranges from about 5% to 45%, 10% to 30%, 10% to 40%, 15% to 35%, 15% to 30%, 15% to 25%, 15% to 20%, or about 18%, or 10.4%, 18.2%, or 41.7%, and in which the pDNA concentration (w/v) in the nucleic acid solution ranges from about 2% to 20%, 4% to 18%, 5% to 15%, 6% to 16%, 6% to 14%, 6% to 12%, 6% to 10%, 6% to 8%, 7% to 8%, or about 8%, or 4.4%, 7.7%, or 17.7%. In any of these embodiments, equal volumes of the solutions containing PEI and pDNA can be combined to form transfection cocktail. In any of these embodiments, the PEI and pDNA can be dissolved or diluted in F17 medium, optionally supplemented with 10 mM Glutamax and 0.2% Pluronic F-68. In any of these embodiments, the host cells can be HEK293 cells, or derivatives thereof, or other cells, and the AAV vector can comprise an AAV9 capsid, or another capsid.

In some embodiments, the methods and systems of the disclosure for continuous transfection of cells can be used or configured to efficiently produce AAV vectors at large scale (for example, in cell culture volumes of at least or about 100 L, 500 L, 1000 L, 2000 L, 5000 L, or more before transfection) by transfecting host cells with transfection cocktail in an amount of at least or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 percent, or more of the cell culture volume or mass before transfection, or some other value between, or range comprising, any of the foregoing specifically enumerated values, for example, about 10% to 60%, 15% to 55%, 20% to 50%, 25% to 45%, 30% to 40%, 30% to 38%, 30% to 36%, 31% to 34%, 32% to 33%, or about 33%, 14.26%, 32.65%, or 57.21% of the cell culture volume or mass before transfection. In any of these embodiments, the host cells can be HEK293 cells, or derivatives thereof, or other cells, and the AAV vector can comprise an AAV9 capsid, or another capsid.

In some embodiments, the methods and systems of the disclosure for continuous transfection of cells can be used or configured to produce AAV vectors by transfecting host cells at large scale (for example, in cell culture volumes of at least or about 100 L, 500 L, 1000 L, 2000 L, 5000 L, or more before transfection) and at high viable cell densities per milliliter (vc/mL) culture at the time of transfection, for example, of at least or about 1×10⁶, 2×10⁶, 3×10⁶, 4×10⁶, 5×10⁶, 6×10⁶, 7×10⁶, 8×10⁶, 9×10⁶, 10×10⁶, 11×10⁶, 12×10⁶, 13×10⁶, 14×10⁶, 15×10⁶, 16×10⁶, 17×10⁶, 18×10⁶, 19×10⁶, 20×10⁶, 21×10⁶, 22×10⁶, 23×10⁶, 24×10⁶, 25×10⁶, 26×10⁶, 27×10⁶, 28×10⁶, 29×10⁶, 30×10⁶, 35×10⁶, 40×10⁶, 45×10⁶, or 50×10⁶ vc/mL, or more, or some other value between, or range comprising, any of the foregoing specifically enumerated values, for example, about 10×10⁶ to 6 vc/mL, 15×10⁶ to 25×10⁶ vc/mL, or 16×10⁶ to 24×10⁶ vc/mL, or about 18×10⁶±0.2 vc/mL. In any of these embodiments, the host cells can be HEK293 cells, or derivatives thereof, or other cells, and the AAV vector can comprise an AAV9 capsid, or another capsid.

In some embodiments, the methods and systems of the disclosure for continuous transfection of cells can be used or configured to produce AAV vectors at high titer by transfecting host cells at large scale (for example, in cell culture volumes of at least or about 100 L, 500 L, 1000 L, 2000 L, 5000 L, or more before transfection). AAV vector titer can be determined using any method known in the art, embodiments of which include quantitative PCR assays that detect AAV ITR sequences, transgene sequences, or some other sequence that is uniquely present in the AAV vector genome. Thus, in some embodiments, AAV vectors can be produced both at large scale and at titers of vector genomes (or genome copies) per mL of cells in culture after transfection that are at least about 1×10⁹, 1×10¹⁰, 1×10¹¹, 1.5×10¹¹, 2×10¹¹, 2.5×10¹¹, 3×10¹¹, 3.5×10¹¹, 4×10¹¹, 4.5×10¹¹, 5×10¹¹, 5.5×10¹¹, 6×10¹¹, 6.5×10¹¹, 7×10¹¹, 7.5×10¹¹, 8×10¹¹, 8.5×10¹¹, 9×10¹¹, 9.5×10¹¹, 1×10¹², 1.25×10¹², 1.5×10¹², 1.75×10¹², 2×10¹², 2.25×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², or 1×10¹³ vg/mL cells, or more, or some other value between, or range comprising, any of the foregoing specifically enumerated values. In any of these embodiments, the host cells can be HEK293 cells, or derivatives thereof, or other cells, and the AAV vector can comprise an AAV9 capsid, or another capsid.

In some embodiments, the methods and systems of the disclosure for continuous transfection of cells can be used or configured to produce AAV vectors with a high proportion of full capsids (i.e., those containing a complete genome) (or conversely, a low percentage of only partially full capsids) by transfecting host cells at large scale (for example, in cell culture volumes of at least or about 100 L, 500 L, 1000 L, 2000 L, 5000 L, or more before transfection). The proportion of full capsids can be estimated using any method known in the art, embodiments of which include purifying AAV vectors, such as by size exclusion chromatography, measuring UV absorbance at two wavelengths (for example, with a spectrophotometer), 260 nm and 280 nm, and then calculating the A260/A280 value. Thus, in some embodiments, AAV vectors can be produced both at large scale and in purified form with A260/A280 values of at least about 0.4, 0.6, 0.7, 0.8, 0.9, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.3, 1.4, 1.5, 1.6, 1.7, or 1.8, or more, or some other value between, or range comprising, any of the foregoing specifically enumerated values. Using other methods familiar to those of ordinary skill in the art, the percentage of vectors that are only partially full (where lower values are desirable) can be measured. Thus, in some embodiments, AAV vectors can be produced both at large scale and in purified form in which the percentage of non-full capsids is less than or about 60%, 55%, 50%, 45%, 40%, 35%, 25%, 20%, 15%, 10%, or 5%, or less, or some other value between, or range comprising, any of the foregoing specifically enumerated values. In any of these embodiments, the host cells can be HEK293 cells, or derivatives thereof, or other cells, and the AAV vector can comprise an AAV9 capsid, or another capsid.

In some embodiments, the methods and systems of the disclosure for continuous transfection of cells can be used or configured to produce AAV vectors at large scale (for example, in cell culture volumes of at least or about 100 L, 500 L, 1000 L, 2000 L, 5000 L, or more before transfection) at titers of at least about 1×10⁹, 1×10¹⁰, 1×10¹¹, 1.5×10¹¹, 2×10¹¹, 2.5×10¹¹, 3×10¹¹, 3.5×10¹¹, 4×10¹¹, 4.5×10¹¹, 5×10¹¹, 5.5×10¹¹, 6×10¹¹, 6.5×10¹¹, 7×10¹¹, 7.5×10¹¹, 8×10¹¹, 8.5×10¹¹, 9×10¹¹, 9.5×10¹¹, 1×10¹², 1.25×10¹², 1.5×10¹², 1.75×10¹², 2×10¹², 2.25×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², or 1×10¹³ vg/mL cells after transfection, or more, or some other value between, or range comprising, any of the foregoing specifically enumerated values, and that in purified form have A260/A280 values of at least about 0.4, 0.5, 0.6, 0.7, 0.8, 0.9, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.3, 1.4, 1.5, 1.6, 1.7, or 1.8, or more, or some other value between, or range comprising, any of the foregoing specifically enumerated values. In any of these embodiments, the host cells can be HEK293 cells, or derivatives thereof, or other cells, and the AAV vector can comprise an AAV9 capsid, or another capsid.

In some embodiments, the methods and systems of the disclosure for continuous transfection of cells can be used or configured to produce AAV vectors at large scale (for example, in cell culture volumes of at least or about 100 L, 500 L, 1000 L, 2000 L, 5000 L, or more before transfection) at titers of at least about 1×10⁹, 1×10¹⁰, 1×10¹¹, 1.5×10¹¹, 2×10¹¹, 2.5×10¹¹, 3×10¹¹, 3.5×10¹¹, 4×10¹¹, 4.5×10¹¹, 5×10¹¹, 5.5×10¹¹, 6×10¹¹, 6.5×10¹¹, 7×10¹¹, 7.5×10¹¹, 8×10¹¹, 8.5×10¹¹, 9×10¹¹, 9.5×10¹¹, 1×10¹², 1.25×10¹², 1.5×10¹², 1.75×10¹², 2×10¹², 2.25×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², or 1×10¹³ vg/mL cells after transfection, or more, or some other value between, or range comprising, any of the foregoing specifically enumerated values, and which in purified form the percentage of non-full capsids is less than or about 60%, 55%, 50%, 45%, 40%, 35%, 25%, 20%, 15%, 10%, or 5%, or less, or some other value between, or range comprising, any of the foregoing specifically enumerated values. In some embodiments, the host cells are HEK293 cells, or derivatives thereof, or other cells, and the AAV vector can comprise an AAV9 capsid, or another capsid.

In some embodiments, the methods and systems of the disclosure for continuous transfection of cells can be used or configured to produce AAV vectors at large scale (for example, in cell culture volumes of at least or about 100 L, 500 L, 1000 L, 2000 L, 5000 L, or more before transfection) and at viable cell densities of at least or about 10×10⁶, 15×10⁶, 20×10⁶, 25×10⁶, 30×10⁶, 40×10⁶, or 50×10⁶ vc/mL, or a range comprising any of the foregoing specifically enumerated values, for example, about 10×10⁶ to 30×10⁶ vc/mL, 15×10⁶ to 25×10⁶ vc/mL, or 16×10⁶ to 24×10⁶ vc/mL, where the cells are transfected with transfection cocktail incubated for 20, 15, 10, 5, 4, 3, 2, or 1 minute or less, where a volume (or mass) of the transfection cocktail at least 10%, 20% or 30% of the volume (or mass) of the cell culture volume before transfection is added to the cells in 90, 80, 70, 60, 50, 40, 30, 20, 10, or 5 minutes or less, and where the Reynolds number Re associated with the flow of transfection cocktail does not exceed a value of 3500 or 4000. In any of these embodiments, the transfection reagent can be PEI and the nucleic acid can be plasmid DNA, and the transfection cocktail can be prepared using a sufficient amount of PEI and pDNA such that the cells are transfected with greater than 0.25 μg pDNA per 10⁶ viable cells, and the mass ratio of PEI to pDNA is at least 1. In any of these embodiments, use of the methods or systems for transfection of the disclosure can be effective to produce recombinant AAV vector with a titer of at least 1×10⁹, 1×10¹⁰, 1×10¹¹, 2×10¹¹, 3×10¹¹, 4×10¹¹, 5×10¹¹, 6×10¹¹, 7×10¹¹, 8×10¹¹, 9×10¹¹, or 1×10¹² vg/mL cells after transfection and, when purified, an A260/A280 ratio of at least 1.0. In any of these embodiments, the host cells can be HEK293 cells, or derivatives thereof, or other cells, and the AAV vector can comprise an AAV9 capsid, or another capsid.

In some embodiments, the methods and systems of the disclosure for continuous transfection of cells can be used or configured to produce AAV vectors by transfecting host cells at a viable cell density of about 18×10⁶ vc/mL in a culture volume of at least 1000 L (before transfection) with transfection cocktail that is incubated for about 135 seconds before being added to the cells, and which contains sufficient plasmid DNA that cells are transfected with about 0.75 μg DNA per 10⁶ viable cells and sufficient PEI that the mass ratio of PEI to pDNA is about 2.2. In some of these embodiments, the system for continuous transfection is configured so that the value of Reynold's number for the flow of transfection cocktail within the system is less than 4000 or 3500. In some of these embodiments, the total volume of cocktail that is used for transfection is about 33% of the pre-transfection volume of the cells. In some of these embodiments, equal volumes of a solution containing PEI at a concentration of about 18-19% (w/v) and a solution containing plasmid DNA at a concentration of about 7-8% (w/v) are mixed to form transfection cocktail. In some of these embodiments, the addition time for substantially the entire volume of transfection cocktail to the cells is about 30 minutes. In any of these embodiments, PEI can be linear PEI, such as linear fully depropionylated PEI, such as 40 kDa linear fully depropionylated PEI. In any of these embodiments, the PEI and pDNA can be dissolved or diluted in F17 medium, optionally supplemented with 10 mM Glutamax and 0.2% Pluronic F-68. In any of these embodiments, the DNA can include three different types of plasmids, one containing sequences for expressing viral helper factors, one containing AAV rep and cap genes, and one containing an AAV vector genome containing a therapeutic transgene. In any of these embodiments, use of the methods or systems are effective to produce AAV vector with a titer of at least 1×10⁹, 1×10¹⁰, 1×10¹¹, 2×10¹¹, 3×10¹¹, 4×10¹¹, 5×10¹¹, 6×10¹¹, 7×10¹¹, 8×10¹¹, 9×10¹¹, or 1×10¹² vg/mL cells after transfection and, when purified, an A260/A280 ratio of at least 1.0. In any of these embodiments, the host cells can be HEK293 cells, or derivatives thereof, or other cells, and the AAV vector can comprise an AAV9 capsid, or another capsid.

### Systems for Transfecting Host Cells

The disclosure additionally provides systems useful for carrying out the methods of transfection disclosed herein. Such systems provide means for containing transfection reagent in solution, means for containing nucleic acid in solution, means for mixing transfection reagent and nucleic acid solutions together, and means for containing host cells to be transfected. Systems can further comprise means for fluid communication between and among the various containment means and the mixing means.

Systems of the disclosure comprise means for containing transfection reagent in solution as well as means for containing nucleic acid in solution (solution containment means). Solution containment means can be any container suitable for containing solutions that will come into contact with cells, including, for example, vessels, reservoirs, bottles, plastic bags (such as WAVE Bioreactor™), carboys, tanks, or single use mixers (SUM), with others possible. Solution containment means may have inlet and/or outlet openings or ports to allow, for example, gas exchange, and introduction and/or exit of fluids, such as transfection reagent and nucleic acid solutions, or mounting of probes. Solution containment means can be made from any material suitable for containing solutions that will come into contact with cells, including for example, glass, rigid or pliable plastics, or metal alloys (such as stainless steel). Exemplary plastics include polyamide, polycarbonate, polyethylene (including low density polyethylene (LDPE)), polyethersulfone, polypropylene, polytetrafluorethylene, polyvinyl chloride, cellulose acetate, ethylene vinyl acetate, ethylene vinyl alcohol (EVOH), nylon, and/or combinations of any of the foregoing, with others possible. Solution containment means can be sealed or open to the atmosphere, although if open can include filters to prevent contamination. Control means for controlling parameters such as temperature, pH, gas content, pressure and mixing of the contents of solution containment means can be employed if desired. Solution containment means can be provided with means for mixing the contents, such as a motor-driven shaft-mounted stir bar, or the like, or an impulse mixer using a pulsing disc, or some other mixing technology. Solution containment means can further be provided or used in conjunction with means for monitoring the volume of solution contained therein. Thus, for example a graduated scale can be included with solution containment means calibrated to the volume inside, or a mechanical or electronic scale could be placed under the solution containment means to monitor changes in weight, which can be correlated with volume of the fluid inside.

Solution containment means can be of any suitable volume. In some embodiments, solution containment means can hold a maximum of at least about 1, 5, 10, 20, 30, 50, 100, 200, 250, 300, 400, 500, 1000, 1500, 2000, 2500, 3000, 3500, 4000, 4500, 5000, 10000 liters, or more, or some other value between or range comprising any of the foregoing specifically enumerated values.

Containment means for containing the transfection reagent solution and the nucleic acid solution can be of the same type or different types. In some embodiments, containment means for the different solutions can be integral, that is, part of one physical unit, but with separate reservoirs or chambers for containing the separate solutions. In other embodiments, containment means for the different solutions are physically separate. Systems can have one containment means for each of the transfection reagent and nucleic acid solutions (thus, a total of two if physically separated), or can have a plurality of such containment means for each of the different types of solutions, which may be the same or different numbers for each solution.

Systems of the disclosure further comprise means for mixing together previously separated transfection reagent and nucleic acid solutions. In some embodiments, mixing means is an element or component of a system where separate solutions of transfection reagent and nucleic acid first encounter each other in the system and start to mix together, even though complete mixing may not always or even usually occur in the mixing means. Instead, with respect to such embodiments, mixing may continue toward completion in other aspects of the system, including for example, in fluid communication means lying downstream of the mixing means, before addition to cells. In other embodiments, mixing means is effective to completely or nearly completely mix transfection reagent and nucleic acid solutions, forming transfection cocktail, before it exits mixing means toward cell containment means.

In some embodiments, mixing means can have moving parts, examples of which include stirrers, such as motorized stirrers having a shaft to which is attached ribbons, blades, paddles, a propeller or the like, or stirrers lacking shafts, such as magnetic stir bar paired with a magnetic or electromagnetic driver, or impulse mixer using a pulsing disc. Other examples include a stator paired with a rotor, a bubbler (which introduces air or other gas at or toward the bottom of a volume of liquid forming bubbles which, as they rise, displace and agitate the liquid causing it to intermix), or mixers that employ sound waves to impart kinetic energy to liquids resulting in their mixture, examples of which include resonant acoustic mixers and ultrasonic mixers. Mixing means can also include static mixers which lack moving parts but contain elements that continuously disturb fluid flowing over, by or past them in a way to cause mixing. Examples of static mixers include plate or wafer type static mixers, and housed-element static mixers, which having a housing and one or more baffles, which can have a variety of configurations, such as helices or flat angled blades. Additional examples of static mixers include low pressure drop or lower pressure drop static mixers, interfacial surface generator static mixers, flow division static mixers, and static radial mixers. Systems can comprise a single mixing means (and any associated mixing containment means, as described below) or a plurality of such mixing means (and any associated mixing containment means), which can be of the same or different types.

Mixing means can be used in conjunction with a further containment means (mixing containment means), such as a vessel, bottle, tank, container or chamber, meant to temporarily hold or store the transfection reagent and nucleic acid solutions while they are being mixed together, whether fully or partially. Such containment means can be chosen or designed to work with the mixing means. For example, a bottle, tank or other container can be designed to accommodate a motor-driven stirrer, or mounted to a motorized platform that shakes or agitates the container's contents. In another example, a thick-walled pliable plastic bag (such as WAVE Bioreactor™) can serve as the container, which is mounted to a platform that rocks or rotates. Mixing containment means can include openings or ports to serve as inlets through which liquids (e.g., transfection reagent and nucleic acid solutions) to be mixed can be introduced, as well outlets through which the mixture (e.g., transfection cocktail) can exit. If mixing does not use a continuous process, the same opening or port can serve as inlet and outlet. Mixing containment means can be sealed or open to the atmosphere, although if open can include filters to prevent contamination. Means for controlling temperature of the contents of the mixing containment means can be employed if desired. In some embodiments, the housing of a static mixer serves as the mixing containment means, being a location in a system where mixing occurs. Mixing containment means can be made from a variety of materials suitable for containing solutions that will come into contact with cells, including glass, plastics and metal alloys, such as stainless steel. Exemplary plastics include polyamide, polycarbonate, polyethylene (including low density polyethylene (LDPE)), polyethersulfone, polypropylene, polytetrafluorethylene, polyvinyl chloride, cellulose acetate, ethylene vinyl acetate, ethylene vinyl alcohol (EVOH), nylon, and/or combinations of any of the foregoing, with others possible.

According to some non-limiting embodiments, the mixing means is a hollow element with multiple tube-like arms that project from at least one junction where the arms meet and join to permit fluid communication between or among the joined arms. Transfection reagent and nucleic acid solutions flow under pump pressure or gravity through separate arms into the hollow element where the solutions meet, begin to mix and then exit as transfection cocktail through at least one other arm. In some embodiments, a hollow element is made of one piece, but can also be made of multiple sub-elements. In some embodiments, the hollow element mixing means includes interior elements, such as baffles, that disturb fluid flow within and thereby enhance mixing of the solutions. In some embodiments, the hollow element is integral with fluid communication means and in other embodiments is a discrete element that is connected via connectors, fittings, seals or the like to fluid communication means. In the latter embodiments, the arms of the hollow element can be same or different lengths. In some embodiments, the arms of the hollow element have circular cross section, whereas in other embodiments, the cross section is some other shape, such as elliptical, square, rectangular, triangular, hexagonal, etc., and the inner dimensions of the several arms can be the same or different.

The interior dimensions of hollow elements can be of any suitable size. In some embodiments, the arms of the hollow element have a cross-sectional inner dimension (such as inner diameter of the bore or lumen of a circular cross-section) of at least or about 0.5, 0.8, 1.6, 3.2, 4.8, 6.4, 8, 0.5, 0.8, 1.6, 3.2, 4.8, 6.4, 8, 9.6, 6.4, 9.6, 12.7, 15.9, 8, 12, 16, 9.6, 12.7, 15.9, 19, 25.4 millimeters, or more, or some other value between or range comprising any of the foregoing specifically enumerated values.

In some embodiments, a hollow element has two inlets for the transfection reagent and nucleic acid solutions and one outlet for transfection cocktail. In this embodiment, inlets can be connected to fluid communication means (described further below) leading from solution containment means separately containing transfection reagent and nucleic acid solutions (one inlet for each respectively), and the outlet can be connected to fluid communication means leading to cell containment means (described further below). In other embodiments, however, a hollow element can contain more than two inlets (usually, but not necessarily an even number) to accommodate connection to multiple sets of solution containment means. For example, two sets of solution containment means could be connected to a hollow member having four inlets total, and one or more outlets. Likewise, a hollow member could have a plurality of outlets to accommodate connection to a plurality of cell containment means via suitable fluid communication means. In some non-limiting embodiments, a hollow element mixing means can have 2, 3, 4, 5, 6 or more inlets, and 1, 2, 3, 4, 5 or more outlets.

The arms of hollow element mixing means can be coplanar, or one or more arms can be angled with respect to the plane formed by the intersection of any two other arms of the same hollow element. The angle of intersection between any two arms of hollow element mixing means can range from greater than 0 degrees to less than 180 degrees, and the angles of intersection between three or more arms can all be equivalent or non-equivalent, or a combination of equivalent and non-equivalent angles. In a non-limiting embodiment, a hollow element mixing means can be T shaped in which the three arms (two of which serve as inlets and one outlet) are coplanar and meet at approximately 90 degrees, whereas in another non-limiting embodiment, the element is Y shaped in which the three arms are coplanar, with two of the arms (serving as inlets) intersecting the third arm (outlet) at equivalent angles that range from greater than 90 degrees to less than 180 degrees. In some non-limiting embodiments, a hollow element mixing means comprises two arms that intersect at an angle of less than 180 degrees, or about 170, 160, 150, 140, 130, 120, 110, 100, 90, 80, 70, 60, 50, 45, 40, 30, 25, 20, 15 degrees, or more than 0 degrees, including all angles between and ranges comprising the foregoing specifically enumerated values.

In certain embodiments, systems can comprise at least a second mixing means in series with the first mixing means. In some embodiment such second mixing means is downstream of the first mixing means, in the sense that transfection cocktail exiting the first mixing means flows, directly or indirectly, into the second mixing means where it undergoes further mixing before exiting such second mixing means as it continues to flow toward the cell containment means. For example, in certain embodiments, the second mixing means can be a hollow element having an inlet arm or port, which thereafter divides or ramifies into two or more tube-like fluid paths that then rejoin downstream at junction where additional mixing occurs, after which transfection cocktail exits via an outlet arm or port.

Systems of the disclosure further comprise means for containing host cells (cell containment means) to be transfected. Examples of cell containment means includes reservoirs, bottles, carboys, tanks, plastic bags, bioreactors of different types, with others possible. Cell containment means can be designed for single use (such as a single-use bioprocess bag), after which the cell containment means is discarded or recycled, or for multiple uses (such as a stainless steel bioreactor tank). Cell containment means can be of different volumes, and made of any material suitable for containing viable host cells including for example, glass, rigid or pliable plastics, or metal alloys (such as stainless steel). Exemplary plastics include polyamide, polycarbonate, polyethylene (including low density polyethylene (LDPE)), polyethersulfone, polypropylene, polytetrafluorethylene, polyvinyl chloride, cellulose acetate, ethylene vinyl acetate, ethylene vinyl alcohol (EVOH), nylon, and/or combinations of any of the foregoing, with others possible.

Because host cells, whether during growth phase, transfection or afterwards, are often highly sensitive to environmental conditions, systems of the disclosure can be configured with additional means to maintain conditions important to cell viability, growth and/or transfection efficiency inside the cell containment means within predetermined ranges. Examples of such environmental conditions include oxygen and CO₂ levels, pH, temperature, and nutrients and other media components required for cellular metabolism, as well as others that will be familiar to those of ordinary skill. The means for maintaining desired environmental conditions can be integral with or separate from the cell containment means. Cell containment means can be fitted with sensors to detect deviations of various environmental parameters from preferred target values or ranges, information that can be acted on automatically or manually to correct the deviations.

In some embodiments, oxygen or other gasses, such as CO₂ to control pH, can be introduced if needed using internal spargers or external gas exchange devices, and temperature can be controlled using heating elements and/or cooling coils immersed in the fluid bathing the cells. Alternatively, cell containment means can have heat added or removed externally, such as by wrapping a tank with a heating pad, or using a double jacketed tank, which allows heated or cooled water to circulate against the inner wall of a bioreactor in which cells are grown or maintained. Cell containment means can also be configured with means for mixing the contents by mechanical (e.g., stirrer, impeller, rotating wall or rocking platform), pneumatic (e.g., vigorous sparging) or hydraulic (e.g., pumping) agitation to ensure homogenous distribution of nutrients, pH, metabolic byproducts, gasses, temperature and the like. Cell containment means can be open to the atmosphere, optionally including filters to prevent contamination, but can be sealed if desired and even pressurized to increase the amount of gasses, such as oxygen, that are dissolved in the fluid bathing the cells, and/or to prevent foaming. Systems can also be configured with perfusion means, internal or external to the cell containment means, for retaining cells while allowing removal of cell waste products and depleted media and addition of fresh media or other components needed for optimal cell growth and/or productivity. Non-limiting examples of perfusion means include a hollow fiber filtration apparatus, such as a tangential flow and alternating tangential flow filtration apparatus, others being possible, such as packed bed bioreactors and fluidized bed bioreactors.

Cell containment means may have one or more inlet and/or outlet openings, ports or drains to allow, for example, gas exchange, the introduction and removal of fluids (such as transfection cocktail, new or old media, media supplements, buffers, anti-foaming agents, antibiotics or other drugs), or the insertion of sensor probes. Such openings, ports or drains can be located in various locations, such as at the top, bottom or sides of the cell containment means. Inlet and outlet openings, ports or drains can be optionally be fitted with valves to control the direction of gas or fluid flow, if desired.

Cell containment means can be of any suitable volume. In some embodiments, cell containment means can hold a maximum of at least our about 1, 5, 10, 20, 30, 50, 100, 200, 250, 300, 400, 500, 1000, 1500, 2000, 2500, 3000, 3500, 4000, 4500, 5000, 10000 liters, or more, or some other value between or range comprising any of the foregoing specifically enumerated values.

Systems of the disclosure can further comprise means of fluid communication, including but not limited to (i) from the means for containing the transfection reagent and nucleic acid solutions to the mixing means (and any associated mixing containment means) to allow the flow of the solutions from the solution containment means to the mixing means (and any associated mixing containment means), and (ii) from the mixing means (and any associated mixing containment means) to the cell containment means to allow the flow of transfection cocktail from the mixing means (and any associated mixing containment means) to the cell containment means. Transfection cocktail within the latter fluid communication means may continue to mix as it flows toward the cell containment means. In addition, the flow rate (which can be related to pump rate) can be adjusted, in conjunction with design choices relating to overall length and cross sectional area of the fluid communication means, to result in a predetermined total mixing or incubation time starting when transfection cocktail first forms and ending when that same portion is added to host cells for purposes of transfection.

In some embodiments, the fluid communication means is a tube, hose or pipe, which can be made of any material suitable for containing solutions that will come into contact with cells, such as glass, plastics or metal alloys, such as stainless steel. Exemplary plastics include polyamide, polycarbonate, polyethylene (including low density polyethylene (LDPE) and linear low density polyethylene (LLDPE)), polyethersulfone, polypropylene, polytetrafluorethylene (PTFE), polyvinyl chloride, polyurethane, cellulose acetate, ethylene vinyl acetate, ethylene vinyl alcohol (EVOH), fluorinated ethylene propylene (FEP), perfluoroalkoxy (PFA), polyvinylidene fluoride (PVDF), nylon, silicone, and/or combinations of any of the foregoing, with others possible. Fluid communication means for use with the systems of the disclosure can be single use or multi-use.

Fluid communication means, such as tubes, hoses or pipes, can be attached or connected to other components of the system, such as solution containment means, mixing means (and any associated mixing containment means), and cell containment means, at inlet or outlet ports, as the case may be, in any leak-resistant manner familiar to those of ordinary skill, such as by quick connectors, couplings, screw joints, friction or compression fittings, seals, welds, and the like. Optionally, fluid communication means can include or be fitted with valves, clamps or the like, that prevent undesired fluid flow, as well as filters to remove particles above a certain size, such as contaminants, including microorganisms.

Systems of the disclosure can have any number of individual fluid communication means. According to certain embodiments, a single fluid communication means, such as a tube, hose or pipe, connects each solution containment means and the mixing means (and any associated mixing containment means). In other embodiments, a plurality of fluid communication means connects each solution containment means and the mixing means (and any associated mixing containment means), which can be the same or a different number. According to certain embodiments, a single fluid communication means, such as a tube, hose or pipe, connects the mixing means (and any associated mixing containment means) and the cell containment means. In other embodiments, a plurality of fluid communication means connects the mixing means (and any associated mixing containment means) and the cell containment means. According to an exemplary non-limiting embodiment, a system can comprise one fluid communications means from each of two solution containment means to a mixing means, and then one additional fluid communication means from the mixing means to cell containment means, for a total of three fluid communication means in the system. Other systems could have different total number of individual fluid communication means, however.

In some embodiments, fluid communication means, such as a tube, hose or pipe, can have circular cross section, whereas in other embodiments, the cross section is some other shape, such as elliptical, square, rectangular, triangular, hexagonal, etc. The internal dimensions of fluid communication means can be of any suitable size. In some embodiments, fluid communication means has a cross-sectional inner dimension (which in the case of a circular cross section would be the diameter of the bore or lumen) of at least or about 0.5, 0.8, 1.6, 3.2, 4.8, 5, 6, 6.4, 7, 8, 9, 9.6, 10, 11, 12, 12.7, 13, 14, 15, 15.9, 16, 17, 18, 19, 20, 21, 22, 23, 24, 25, 25.4, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 90, 95, 100 millimeters (mm), or more, or some other value between or range comprising any of the foregoing specifically enumerated values. In some embodiments, fluid communication means between the mixing means and cell containment means downstream is a pipe or tube with circular cross section and an inner diameter ranging from about 0.5 to 7.5 centimeters (cm), to 5 cm, 0.5 to 4 cm, 0.5 to 3 cm, 0.5 to 2.5 cm, 0.5 to 2 cm, 0.5 to 1.5 cm, 0.5 to 1 cm, 0.75 to 7.5 cm, 0.75 to 5 cm, 0.75 to 4 cm, 0.75 to 3 cm, 0.75 to 2.5 cm, 0.75 to 2 cm, 0.75 to 1.5 cm, to 1 cm, 1 to 7.5 cm, 1 to 5 cm, 1 to 4 cm, 1 to 3 cm, 1 to 2.5 cm, 1 to 2 cm, 1 to 1.5 cm, 1.5 to 7.5 cm, 1.5 to 5 cm, 1.5 to 4 cm, 1.5 to 3 cm, 1.5 to 2.5 cm, or 1.5 to 2 cm.

The wall of fluid communication means can have any suitable thickness. In some embodiments, the thickness of the wall of fluid communication means, such as those of tubes, hoses or pipes, can be at least or about 0.5, 1, 1.5, 2, 2.5, 3, 3.5, 4, 4.5, 5, 5.5, 6, 6.5, 7, 7.5, 8, 8.5, 9, 9.5, 10 millimeters, or more, or some other value between or range comprising any of the foregoing specifically enumerated values. Within a system, the dimensions of any fluid communication means within the system can be the same or different as other fluid communication means within the same system.

Fluid communication means of the system, for example, tubes, hoses or pipes, can have different lengths, and in systems comprising more than one fluid communication means, each such fluid communication means can have length that is different from others in the same system. Fluid communication means can be of any suitable length. In some embodiments, length of a fluid communications means is at least or about 0.05, 0.1, 0.5, 1, 2, 3, 4, 5, 10, 15, 20, 25, 30, 40, 45, 50, 55, 60, 65, 70, 75, 80, 85, 90, 95, 100, 110, 120, 130, 140, 150, 160, 170, 180, 190, 200 feet or meters, or more, or some other value between or range comprising any of the foregoing specifically enumerated values. In some embodiments, the length of fluid communication means between mixing means and cell containment means is longer than that of fluid communications means between solution containment means and mixing means.

In some embodiments, fluid communication means, such as a tube, hose or pipe, can be configured, for at least a portion of its overall length, as one or more coils (for example, 1, 2, 3, 4, 5 or more coils), each of which can be a flat coil, a helical coil (as around a cylinder or cone, and in a single layer or wound orthocyclically), a wound toroidal coil, or some other coil configuration. The fraction of the total length of fluid communication means that is coiled can be any suitable fraction. In some embodiments, the percent of the overall length of a fluid communication means that is coiled is at least or about 2, 3, 4, 5, 6, 7, 8, 9, 10, 15, 20, 25, 30, 35, 45, 50, 55, 60, 65, 70, 75, 80, 85, 90, or 95 percent, or some other value between or range comprising any of the foregoing specifically enumerated values. Each coil can have a coil radius (average or constant), which in some embodiments is at least or about 1, 5, 10, 15, 20, 25, 30, 35, 45, 50, 60, 70, 80, 90, 100, 110, 120, 130, 140, 150 centimeters or inches, or more, or some other value between or range comprising any of the foregoing specifically enumerated values.

Systems of the disclosure can further comprise means for pumping (pump means) fluids through the system from the solution containment means to the mixing means (and any associated mixing containment means) and thereon to cell containment means. In certain embodiments the pump means is a peristaltic pump, diaphragm pump (including air-operated diaphragm pump, double-diaphragm pump, diaphragm metering pump, or quaternary diaphragm pump), lobe pump (including rotary lobe pump), gear pump, piston pump (including rotary piston pump), eccentric screw pump, positive displacement pump (including rotating positive displacement pump), centrifugal pump, any of which can be single use pumps or multi-use pumps. In other embodiments, systems of the disclosure can rely on gravity to cause fluid flow through a portion or even the entire system to effect mixing of transfection reagent and nucleic acid solutions and thereafter transfection of host cells. Systems of the disclosure can have any number of pump means, for example 1, 2, 3, 4, 5, or more pump means. Pump means can be configured to operate functionally with any one or more of the system components, including for example, solution containment means, mixing means (and any associated mixing containment means), cell containment means, and means of fluid communication between any of the system's other components, and can be located internal or external to any of the system components. In an exemplary non-limiting embodiment, pump means can be a peristaltic pump that operates in conjunction with a pliable tube serving as fluid communication means between solution containment means and mixing means. One such pump can operate on more than one such tube or, in other embodiments, each such tube could be provided with its own dedicated peristaltic pump, in which case the system could comprise at least two such pumps. In embodiments having two or more pumps, systems can optionally further comprise controls to regulate and coordinate the rate of pumping from different solution containment means so that approximately constant amounts per time (which can be equal or unequal) of transfection reagent and nucleic acid solution are pumped to mixing means.

According to an exemplary non-limiting embodiment, a system of the disclosure can be configured to include two single use mixers to contain transfection reagent on the one hand and nucleic acid (for example, plasmid DNA) in solution on the other. Leading from each SUM is a pliable plastic tube, a portion of which is mounted to a peristaltic pump (thus, two pumps total). The other end of each tube is then connected to an inlet of a “T” or “Y” connector serving as a static in-line mixer in which the solutions begin to mix. To the outlet of the connector is attached a longer post-mixer plastic tube, which may contain one or more coils along its length, terminating at and connected to a port of a bioreactor. In operation, solutions containing transfection reagent and nucleic acid are added to their respective SUMs (or are prepared in the SUMs). The peristaltic pumps are started and set to desired pump rates, causing the solutions to flow out of the SUMs, through the tube and into the connector, where the solutions encounter each other and begin to mix together, forming transfection cocktail. Exiting the mixer, the cocktail proceeds down the longer tube toward the bioreactor while it continues to mix and incubate, forming particles capable of being taken up by the cells. The length of the tube, in conjunction with its inner diameter and the pump rate, determines the incubation time. After transiting the post-mixer tube, transfection cocktail then enters the bioreactor, where it is mixed with the cells in suspension, resulting in their transfection with the nucleic acid.

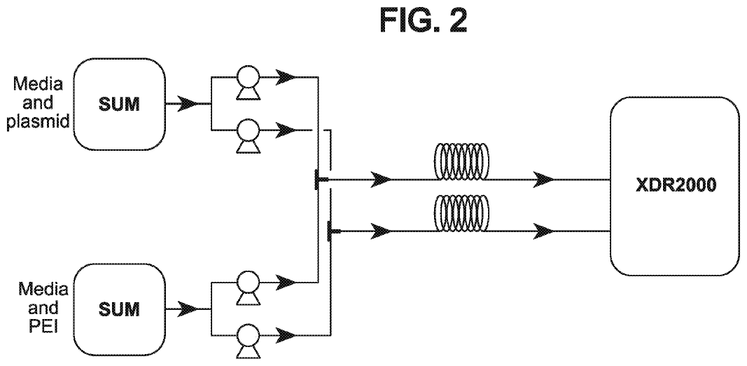

As described above, systems of the disclosure can have a plurality of subcomponents. In some embodiments, for example, a system can include one containment means each for transfection reagent solution, nucleic acid solution, and host cells, while including a plurality of subsystems (such as two or more), each comprising mixing means (and any associated mixing containment means), fluid communication means, and optionally pump means. By including a plurality of such subsystems, systems can be configured to more rapidly deliver a given volume of transfection cocktail to cells without needing to vary transfection cocktail incubation time from a desired predetermined value. A non-limiting example of this embodiment is illustrated in FIG. 2, with other configurations possible.

Systems of the disclosure can be configured, taking into account such variables as pump rate and the dimensions of fluid communication means, to control the incubation time of the transfection cocktail and the time for the total transfection volume to be added to cells (addition time). Total transfection volume is the combined volume of the transfection reagent solution and the nucleic acid solution and is equivalent to the total volume of transfection cocktail to be delivered to cells to be transfected. Total transfection volume depends on variables, such as the volume of cells to be transfected and/or the viable cell density of such cells. Addition time is the time within which it is desired to add the total transfection volume to the cells. Addition time depends on variables, such as the capability of the cell containment means to sufficiently mix and distribute transfection cocktail in the fluid suspending or bathing cells so as to prevent locally toxic concentrations from occurring. Incubation time is the time during which transfection reagent and nucleic acid in solution are in contact forming transfection cocktail, and begins when the two solutions encounter each other and begin mixing in the mixing means, and ends when the transfection cocktail is added to cells in the cell containment means. System parameters to achieve a desired incubation time and addition time can be calculated as follows.

Once total transfection volume and addition time have been determined, the required amounts of transfection reagent solution and nucleic acid solution can be calculated, as well as the flow rate and length of tube (or functionally equivalent fluid communication means) is required to achieve a target incubation time. In some embodiments, each solution is mixed with the other in a 1:1 ratio to form transfection cocktail, although other ratios are possible depending on the concentration of transfection reagent and nucleic acid in their respective solutions. In the case where the two solutions are mixed 1:1, the volume of each solution will be one-half the target total transfection volume. This value is then divided by the addition time to determine the pump rate (volume per time) required for each solution. In system embodiments where each of the two solutions is served by its own pump, this value would be the pump rate of each pump. The total flow rate through the system is then the sum of the pump rates. To calculate the length of tubing (or functionally equivalent fluid communication means) needed to achieve a target incubation time, the desired incubation time is multiplied by the flow rate of the transfection cocktail exiting the mixing means (total flow rate of the system), and then this product is divided by the volume per unit length of tubing. An exemplary set of calculations is shown in Example 6.

Systems of the disclosure can be configured, taking into account such variables as pump rate and the dimensions of fluid communication means, to control whether flow through the system is laminar or turbulent, as expressed by Reynolds number. Reynolds number (Re) is a dimensionless number describing fluid flow, which can be calculated from fluid density (rho (ρ), expressed in units kg/m³), fluid viscosity (mu (μ), expressed in units Pa*s), and linear velocity of the fluid (ν, expressed in units m/s). In the case of fluid flowing through a pipe or similar structure, the formula for Reynolds number is given by:

$$Re = \frac{ρ \cdot v \cdot D}{μ} ,$$

where D is the inner diameter of the pipe in meters. For example, by way of illustration only and not limitation, if a transfection cocktail has density of 1000 kg/m³, viscosity of 1 mPa*s, and velocity of 0.4 m/s through a tube with inner diameter of 1 cm, then the Reynolds number (Re) associated with the flow of such transfection cocktail would be 4000.

In some embodiments, density of transfection cocktail is 997 kg/m 3 and the viscosity of transfection cocktail is 8.90×10⁻⁴ Pa*s (or 0.89 mPa*s), although these values can be different depending on the type of transfection reagent used and the concentrations of such reagent and nucleic acid in solution, as well as the temperature. Thus, in some embodiments, the density of transfection cocktail at 20° C. is about 950, 960, 970, 975, 980, 981, 982, 983, 984, 985, 986, 987, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1025, 1030, 1040, or 1050 kg/m³, or some other value between or some range including and between any of the foregoing values. In some embodiments, the dynamic viscosity of transfection cocktail at 20° C. is about 0.50, 0.55, 0.60, 0.65, 0.70, 0.75, 0.80, 0.81, 0.82, 0.83, 0.84, 0.85, 0.86, 0.87, 0.88, 0.90, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, 1.10, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 6, 7, 8, 9, or 10 mPa*s, or some other value between, or some range including and between, any of the foregoing values.

The linear velocity of transfection cocktail in the system can be any suitable linear velocity. In some embodiments, the linear velocity of transfection cocktail in fluid communication means of the systems of the disclosure, such as tube or pipe connecting the mixing means with cell containment means downstream, is at least or about 0.001, 0.005, 0.01, 0.03, 0.04, 0.05, 0.06, 0.07, 0.08, 0.09, 0.10, 0.11, 0.12, 0.13, 0.14, 0.15, 0.16, 0.17, 0.18, 0.20, 0.21, 0.22, 0.23, 0.24, 0.25, 0.26, 0.27, 0.28, 0.29, 0.30, 0.31, 0.32, 0.33, 0.34, 0.35, 0.37, 0.38, 0.39, 0.40, 0.41, 0.42, 0.43, 0.44, 0.45, 0.46, 0.47, 0.48, 0.49, 0.50, 0.55, 0.60, 0.70, 0.75, 0.80, 0.85, 0.90, 0.95, 1, 2, 3, 4, 5, 6, 7, 8, 9, 10, 20, 30, 40, or 50 meters per second (m/s), or more, or some other value between, or some range including and between, any of the foregoing values.

The flow rate of transfection cocktail in the system can be any suitable flow rate. In some embodiments, the flow rate of transfection cocktail in the systems of the disclosure is at least or about 1, 10, 20, 30, 40, 50, 60, 70, 80, 90, 100, 500, 1000, 1500, 2000, 2200, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5500, 6000, 6500, 7000, 7500, 8000, 8500, 9000, 9500, 10000, 10500, 11000, 11500, 12000, 12500, 13000, 13500, 14000, 15000, 15500, 16000, 16500, 17000, 17500, 18000, 18500, 19000, 19500, or 20000, or more, milliliters per minute (mL/min), or some other value between, or some range including and between, any of the foregoing specifically enumerated values.

In some embodiments, flow rate through a pipe or tube with circular cross section can be use converted to the linear velocity of the fluid moving through the pipe or tube at the particular rate of flow using the formula

$$v = \frac{4 \cdot Q}{π \cdot {D}^{2}} ,$$

where ν is the fluid velocity (m/s), Q is the fluid flow rate (m³/s), and D is the inner diameter (m) of the pipe or tube. Thus, for example, if transfection cocktail moves through a tube with 0.5 inch inner diameter at a rate of rate of 5000 mL/min, it is possible to convert units and calculate the velocity of the fluid to be approximately 0.658 m/s through the tube.

In some embodiments, the flow rate of transfection cocktail in the systems of the disclosure can be expressed as mass of the transfection cocktail in grams or kilograms per unit time, such as seconds or minutes. Thus, for example, in some embodiments, the flow rate of transfection cocktail in the systems of the disclosure is at least or about 1, 10, 20, 30, 40, 50, 60, 80, 90, 100, 500, 1000, 1500, 2000, 2200, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5500, 6000, 6500, 7000, 7500, 8000, 8500, 9000, 9500, 10000, 10500, 11000, 11500, 12000, 12500, 13000, 13500, 14000, 15000, 15500, 16000, 16500, 17000, 17500, 18000, 18500, 19000, 19500, or 20000, or more, grams per minute (g/min), or some other value between, or some range including and between, any of the foregoing specifically enumerated values.

Taking into account its density and viscosity, then controlling the rate at which transfection cocktail flows through tubing (or functionally equivalent fluid communication means) of selected inner diameter connecting mixing means and cell containment means (by, for example, controlling the rate at which transfection reagent and nucleic acid solutions are pumped into the mixing means), then the nature of fluid flow, whether laminar or turbulent, can be controlled in terms of Re. In some embodiments, laminar flow is considered to occur below a Re value of 2000, 3000, 4000, or 5000, whereas turbulent flow is considered to occur above these Re values. According to certain embodiments, flow of transfection cocktail in systems of the disclosure has Re that is at least or about 10, 20, 30, 40, 50, 60, 70, 80, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, or more, or some other value between or range comprising any of the foregoing specifically enumerated values. Thus, for example, in some embodiments, methods of the disclosure are performed, and/or systems of the disclosure are designed and implemented, such that the Reynolds number Re associated with flow of transfection cocktail through fluid communication means from the mixing means to the cell containment means does not exceed a value of 4000, or ranges from about 100 to 4000, 200 to 4000, 300 to 4000, 400 to 4000, 500 to 4000, 600 to 4000, 700 to 4000, 800 to 4000, 900 to 4000, 1000 to 4000, 1100 to 4000, 1200 to 4000, 1300 to 4000, 1400 to 4000, 1500 to 4000, 1600 to 4000, 1700 to 4000, 1800 to 4000, 1900 to 4000, 2000 to 4000, 2100 to 4000, 2200 to 4000, about 2300 to 4000, 2400 to 4000, 2500 to 4000, 2600 to 4000, 2700 to 4000, 2800 to 4000, 2900 to 4000, 3000 to 4000, 3100 to 4000, 3200 to 4000, 3300 to 4000, 3400 to 4000, 3500 to 4000, 3600 to 4000, 3700 to 4000, 3800 to 4000, or 3900 to 4000, or some other range.

In some embodiments, for convenience, the density and viscosity of transfection cocktail can be assumed to be the same as water at 20° C. (ρ=997 kg/m³ and μ=1.00 mPa·s, respectively) and the maximum linear velocity of transfection cocktail through fluid communication means in the form of a pipe or tube with circular cross section can be calculated which would cause the Reynolds number associated with the flow to have a value of 4000 or less. Thus, for example, in some embodiments of the methods and systems of the disclosure, if a tube for carrying transfection cocktail from mixing means to cell containment means has inner diameter D through which flows transfection cocktail at velocity ν, Reynolds number Re associated with such flow would not exceed a value of 4000 where D≥0.32 cm and ν≤1.264 m/s, D≥0.64 cm and ν≤0.632 m/s, D≥1.27 cm and ν≤0.316 m/s, D≥1.91 cm and ν≤0.211 m/s, D≥2.54 cm and ν≤0.158 m/s, D≥3.18 cm and ν≤0.126 m/s, D≥3.81 cm and ν≤0.105 m/s, D≥4.45 cm and ν≤0.090 m/s, D≥5.08 cm and ν≤0.079 m/s, D≥5.72 cm and ν≤0.070 m/s, D≥6.35 cm and ν≤0.063 m/s, D≥6.99 cm and ν≤0.057 m/s, D≥7.62 cm and ν≤0.053 m/s, D≥8.26 cm and ν≤0.049 m/s, D≥8.89 cm and ν≤0.045 m/s, D≥9.53 cm and ν≤0.042 m/s, D≥10.16 cm and ν≤0.039 m/s, D≥10.80 cm and ν≤0.037 m/s, D≥11.43 cm and ν≤0.035 m/s, D≥12.07 cm and ν≤0.033 m/s, or where D≥12.70 cm and ν≤0.032 m/s.

Other objects, features and advantages of the present invention will be apparent from the foregoing detailed description. It should be understood, however, that the detailed description and the specific examples that follow, while indicating specific embodiments of the invention, are given by way of illustration only, since various changes, modifications and equivalents within the spirit and scope of the invention will be apparent from the detailed description and examples to those of ordinary skill in the art, and fall within the scope of the appended claims.

Unless otherwise indicated, use of the term “or” in reference to one or more members of a set of embodiments is equivalent in meaning to “and/or,” and does not require that they be mutually exclusive of each other. Unless otherwise indicated, a plurality of expressly recited numeric ranges also describes a range the lower bound of which is derived from the lower or upper bound of any one of the expressly recited ranges, and the upper bound of which is derived from the lower or upper bound of any other of the expressly recited ranges. Thus, for example, the series of expressly recited ranges “10-20, 20-30, 30-40, 40-50, 100-150, 200-250, 275-300,” also describes the ranges 10-50, 50-100, 100-200, and 150-250, among many others. Unless otherwise indicated, use of the term “about” before a series of numerical values or ranges is intended to modify not only the value or range appearing immediately after it but also each and every value or range appearing thereafter in the same series. Thus, for example, the phrase “about 1, 2, or 3,” is equivalent to “about 1, about 2, or about 3.”

All publications and references, including but not limited to articles, abstracts, patents, patent applications (whether published or unpublished), and biological sequences (including, but not limited to those identified by specific database reference numbers) cited herein are hereby incorporated herein by reference in their entirety for all purposes to the same extent as if each individual publication or reference were specifically and individually indicated to be so incorporated by reference. Any patent application to which this application claims priority directly or indirectly is also incorporated herein by reference in its entirety.

Unless otherwise indicated, the examples below describe experiments that were or are performed using standard techniques well known and routine to those of ordinary skill in the art. The examples are illustrative, but do not limit the invention.

### EXAMPLES

### Example 1: Time Dependence of Transfection Efficiency Using Small Scale Bolus Transfection

This example describes small scale experiments to determine the relationship between incubation time of transfection cocktail on the quantity of an AAV vector produced from host cells transfected with a bolus of transfection cocktail.

Three types of plasmids containing the genetic information required to make a recombinant AAV vector for expressing a mini-dystrophin protein were combined in F17 media and samples dispensed into plate wells. The first plasmid (helper plasmid) contained adenoviral helper functions, the second plasmid (transgene plasmid) contained an AAV vector genome including an AAV2 ITR, a muscle-specific enhancer and promoter, a gene encoding a human dystrophin derived mini-dystrophin protein (named Optidys3978), a transcriptional terminator sequence and a second AAV2 ITR, and the third plasmid (rep/cap) contained an AAV2 rep gene and AAV9 cap gene. The plasmids used in this and the other examples are described further in WO 2017/221145. The different plasmids were combined in a mass ratio of 2.0 (helper):1.6 (rep/cap):1.0 (transgene), equivalent to a molar ratio of 0.94:1.93:1.00, respectively, and pDNA and PEI were combined in a mass ratio of 2.2:1. Plasmid stocks (approximately 1 mg/mL) were stored frozen before use. Sufficient pDNA was used so that 1 μg pDNA would be added per 1×10⁶ viable cells, as determined using a Beckman Coulter Vi-Cell XR.

Fully depropionylated linear polyethylenimine (PEI) 40 kDa in F17 media was then added to the samples of plasmids, one sample at a time. Upon adding the PEI, the transfection reagent and plasmid solutions were mixed by pipetting for 10 seconds and then incubated for varying amounts of time to allow complexes containing PEI and pDNA to form. After the incubation, the resulting transfection cocktails (3 mL) were added in a single bolus to Ambr bioreactors (Sartorius) (15 mL capacity; one for each cocktail sample) containing suspension-adapted HEK293 cells at a viable cell density of approximately 18×10⁶ cells/mL. Three hours after addition of transfection cocktail, transfection was quenched by addition of a 1.5 mL bolus of CDM4HEK293 media, followed by incubation for 68-72 hours to allow production of AAV vector, after which the cells were harvested and AAV vector titered using a quantitative PCR (qPCR) assay specific for the AAV ITRs in the vector genomes.

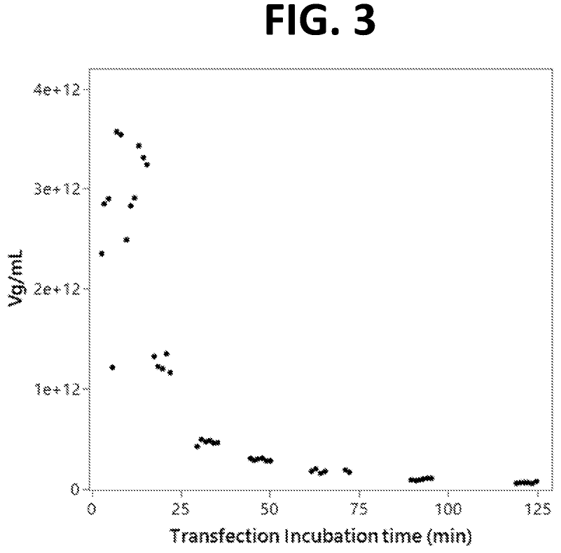

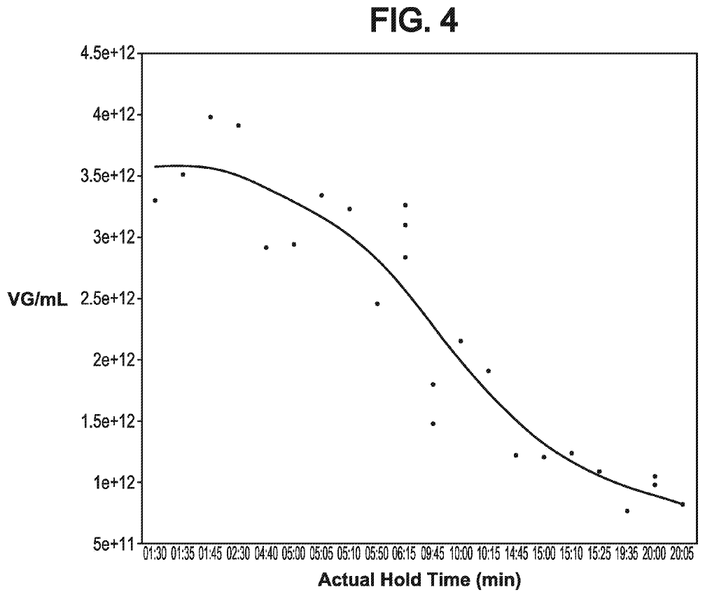

AAV titer (expressed as vector genomes per ML cell culture (vg/ML)) was graphed against the incubation time of the transfection cocktail as shown in FIG. 3. The data show that relatively short transfection cocktail incubation times (about 3-15 minutes) result in high AAV titers whereas incubation times exceeding about 15 minutes result in a substantial decline in AAV production, which plateaus by about 25-30 minutes. A similar experiment was carried out to study the effect on AAV titer of shorter transfection cocktail incubation times, with the results shown in FIG. 4. In this experiment, even very short incubation times of about 1.5 to 2.5 minutes resulted in high AAV titers, whereas incubation time in excess of about 5-6 min resulted in a time-dependent reduction of AAV titer.

### Example 2: Time Dependence of Transfection Efficiency Using Small Scale Continuous Transfection

This example describes small scale experiments to determine the relationship between incubation time of transfection cocktail on the quantity of an AAV vector produced from host cells transfected using a continuous process employing a static in-line mixer to prepare transfection cocktail.

The same types of plasmids, transfection reagent, media and cells were used in these experiments as in Example 1, but transfection was carried out at 1 L scale with a larger volume of cells using a continuous transfection process. Equal volumes of pDNA and PEI solutions were separately prepared in F17 media and dispensed into bottles (one for each solution). About 700 mL cells were transfected with a total volume of transfection cocktail of about 229 mL (32.65% w/v of the cell culture volume before transfection) when the viable cell density in the culture reached approximately 18×10⁶ cells/mL. Sufficient pDNA and PEI were respectively prepared in solution so that 0.75 μg pDNA would be added per 1×10⁶ viable cells and the PEI to pDNA mass ratio in transfection cocktail was 2.2:1. The mass ratios of the plasmids were 2.0 (helper):1.6 (rep/cap):1.0 (transgene), equivalent to molar ratios of 0.94:1.93:1.00, respectively. After all transfection cocktail had been prepared and added to the bioreactor, cells were incubated for 3 hours and then transfection quenched by adding CDM4HEK293 media (13.1% w/v of the cell culture volume before transfection). Cells were then incubated for 68-72 hours to permit AAV vector production, after AAV vector in culture samples was purified and assayed, including titer using a qPCR assay specific for the mini-dystrophin transgene and, after size exclusion chromatography (SEC) purification, the UV absorbance ratio at 260 nm and 280 nm determined with a spectrophotometer as an approximate representation of the proportion of full versus empty capsids (see, e.g., Sommer, J M et al., Mol Ther 7(1):122-8 (2003)). Reynold's number was also calculated as described elsewhere herein.

Transfection was carried out using a system comprising a static in-line mixer. More specifically, the system included two bottles for separately containing the PEI and pDNA in solution. Leading from each bottle was an equal length of flexible plastic tubing (Saint-Gobain C-flex, size 16 (⅛ inch inner dia., ¼ inch outer dia.)), which was inserted through a peristaltic pump (Masterflex; one for each tube) and connected at its end to an inlet of a T fluid connector, so that the end of each of the two tubes met at a 180° angle and at right angles to the outlet. Attached to the outlet was a similar tube leading to a stirred tank glass bioreactor (Broadley-James Bionet) with a total volume of 1 L. The length of the tube from the connector to the bioreactor and pump rates were varied to control both the time for the transfection cocktail to travel from the T connector to the bioreactor (incubation time) and the time to add the total combined volumes of PEI and pDNA solutions as transfection cocktail to the bioreactor (addition time). In these experiments, the solutions containing PEI and pDNA were of equal volume and the rate for each pump were also the same.

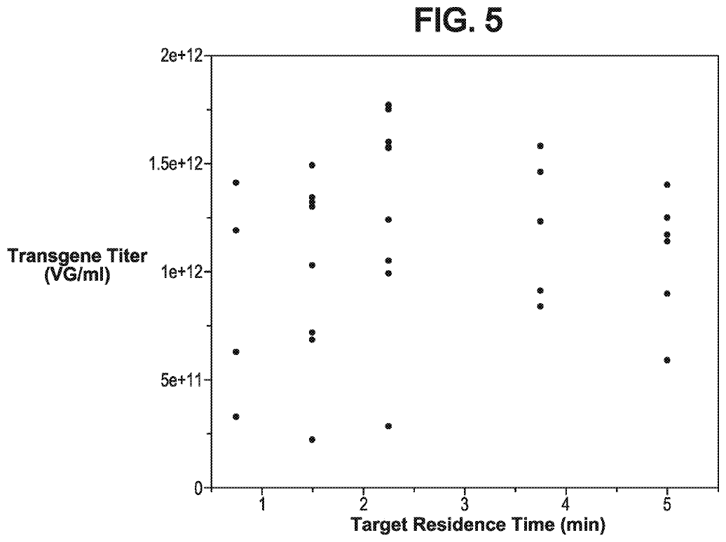

The results from these experiments is summarized in Table 2 and graphically presented in FIG. 5. Although the shortest and longest incubation times tested resulted in high titers of AAV vector, there was a trend in the data indicating that transfection cocktail incubation time of about 2.25 minutes (135 seconds) yielded the highest average AAV vector titer.

TABLE 2

| | | | Total Pump | Reynold's | | AAV Vector |
|---|---|---|---|---|---|---|
| Experiment | Incubation | Addition | Rate | Number | SEC | Titer |
| No. | Time (min) | Time (min) | (mL/min) | (Re) | UV260/UV280 | (vg/mL) |
| 1 | 0.75 | 60 | 3.8 | 25 | 1.14 | 1.41E+12 |
| 2 | 1.5 | 30 | 7.6 | 51 | 1.20 | 1.49E+12 |
| 3 | 1.5 | 30 | 7.6 | 51 | 1.03 | 1.32E+12 |
| 4 | 0.75 | 60 | 3.8 | 25 | 0.91 | 1.19E+12 |
| 5 | 3.75 | 15 | 15.3 | 105 | 1.05 | 1.23E+12 |
| 6 | 5 | 15 | 15.3 | 102 | 0.92 | 1.14E+12 |
| 7 | 5 | 30 | 7.6 | 51 | 1.16 | 1.25E+12 |
| 8 | 2.25 | 45 | 5.1 | 34 | 0.95 | 1.75E+12 |
| 9 | 0.75 | 30 | 15.3 | 25 | 1.16 | 6.27E+11 |
| 10 | 1.5 | 30 | 7.6 | 51 | 1.06 | 1.03E+12 |
| 11 | 5 | 45 | 5.1 | 34 | 0.98 | 5.89E+11 |
| 12 | 3.75 | 60 | 3.8 | 25 | 1.10 | 8.39E+11 |
| 13 | 2.25 | 15 | 15.3 | 102 | 1.12 | 1.24E+12 |
| 14 | 2.25 | 30 | 7.6 | 51 | N/A | 1.60E+12 |
| 15 | 2.25 | 30 | 7.6 | 51 | 1.05 | 1.24E+12 |
| 16 | 5 | 60 | 3.8 | 25 | N/A | 1.17E+12 |
| 17 | 1.5 | 30 | 7.6 | 51 | 1.13 | 1.30E+12 |
| 18 | 3.75 | 30 | 7.6 | 51 | 1.02 | 9.11E+11 |
| 19 | 2.25 | 60 | 3.8 | 25 | 1.06 | 9.92E+11 |
| 20 | 2.25 | 60 | 3.8 | 25 | 1.13 | 1.05E+12 |
| 21 | 0.75 | 15 | 15.3 | 102 | 1.20 | 3.27E+11 |
| 22 | 1.5 | 30 | 7.6 | 51 | 1.10 | 1.34E+12 |
| 23 | 2.25 | 30 | 7.6 | 51 | 1.03 | 1.57E+12 |
| 24 | 3.75 | 30 | 7.6 | 51 | 1.01 | 1.58E+12 |
| 25 | 5 | 30 | 7.6 | 51 | 1.09 | 1.40E+12 |
| 26 | 2.25 | 45 | 5.1 | 34 | 1.13 | 1.58E+12 |
| 27 | 1.5 | 15 | 15.3 | 102 | 1.09 | 7.17E+11 |
| 28 | 2.25 | 15 | 15.3 | 102 | 1.11 | 1.77E+12 |
| 29 | 5 | 60 | 3.8 | 25 | 1.10 | 8.97E+11 |
| 30 | 1.5 | 30 | 7.6 | 51 | 1.15 | 2.22E+11 |
| 31 | 2.25 | 45 | 5.1 | 34 | 1.00 | 2.83E+11 |
| 32 | 1.5 | 45 | 5.1 | 34 | 1.12 | 6.85E+11 |
| 33 | 3.75 | 30 | 7.6 | 51 | 1.10 | 1.46E+12 |

### Example 3: Time Dependence of Transfection Efficiency Using Small Scale Continuous Transfection

This example describes experiments to determine the effect of viable cell density and amount of pDNA on AAV vector titer and SEC UV260/UV280 values.

Experimental design was similar to that in Example 2, except that viable cell density (VCD) was varied, and the system tube lengths and pump rates were held constant to achieve a constant incubation time of 90 seconds and addition time of 30 minutes. Total pump rate was 7.6 mL/min (resulting from the action of two pumps operating at half that rate), tubing length from mixer to bioreactor was 143 cm, and calculated Reynolds number was 57. Because VCD varied while the total amount of pDNA in transfection cocktail was the same as in Example 2 and held constant, the mass of pDNA per million viable cells also varied in these experiments.

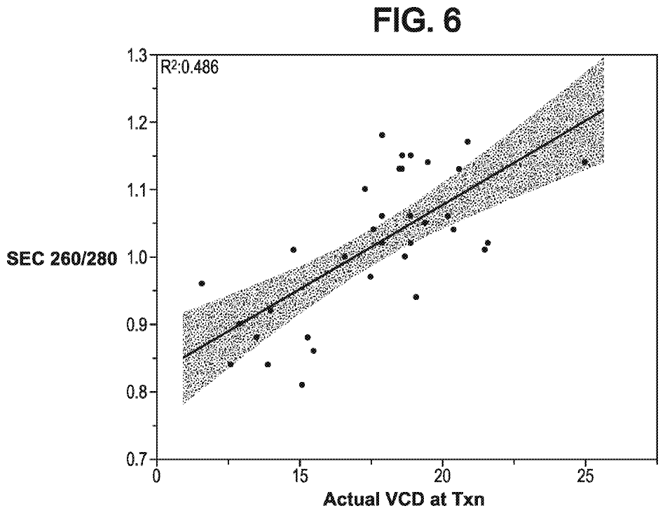

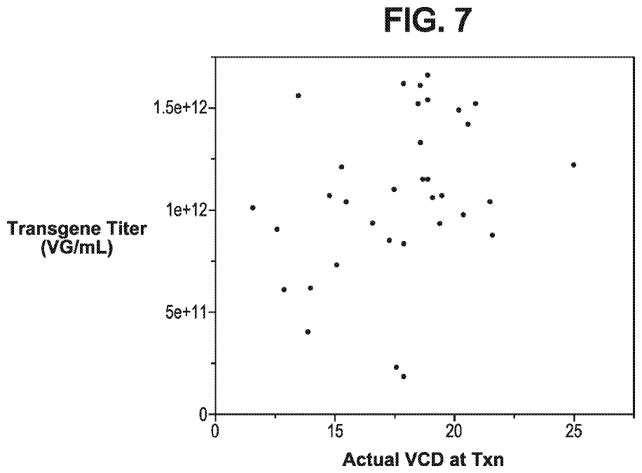

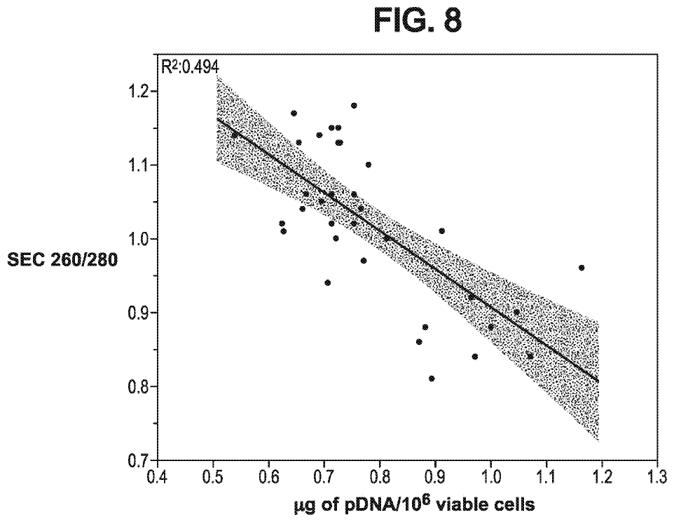

The results from these experiments are summarized in Table 3 and graphically presented in FIGS. 6, 7, and 8. The data demonstrate a positive correlation between viable cell density at transfection and the SEC UV260/UV280 ratio (FIG. 6), indicating that higher VCD favored production of full capsids by the cells. VCD was also weakly positively correlated to AAV vector titer (FIG. 7). Conversely, the quantity of pDNA per million viable cells being transfected was negatively correlated to SEC UV260/UV280 ratio (FIG. 8), indicating that higher quantities of pDNA per cell reduced production of full capsids by the cells, which is generally considered undesirable. While not shown, it was determined that when the quantity of pDNA per million cells was reduced to 0.25 μg, no AAV vector was produced.

TABLE 3

| | VCD at | | SEC | AAV Vector |
|---|---|---|---|---|
| | Transfection | μg pDNA/10⁶ | UV260/ | Titer |
| Experiment No. | (10⁶ cells/mL) | viable cells | UV280 | (vg/mL) |
| 1 | 21.5 | 0.63 | 1.01 | 1.04E+12 |
| 2 | 19.4 | 0.70 | 1.05 | 9.33E+11 |
| 3 | 18.7 | 0.72 | 1.00 | 1.15E+12 |
| 4 | 20.4 | 0.66 | 1.04 | 9.76E+11 |
| 5 | 21.6 | 0.63 | 1.02 | 8.77E+11 |
| 6 | 25.0 | 0.54 | 1.14 | 1.22E+12 |
| 7 | 18.5 | 0.73 | 1.13 | 1.52E+12 |
| 8 | 20.9 | 0.65 | 1.17 | 1.52E+12 |
| 9 | 17.6 | 0.77 | 1.04 | 2.28E+11 |
| 10 | 17.9 | 0.75 | 1.18 | 1.62E+12 |
| 11 | 17.9 | 0.75 | 1.06 | 1.83E+11 |
| 12 | 15.3 | 0.88 | 0.88 | 1.21E+12 |
| 13 | 18.9 | 0.71 | 1.02 | 1.66E+12 |
| 14 | 13.5 | 1.00 | 0.88 | 1.56E+12 |
| 15 | 17.9 | 0.75 | 1.02 | 8.35E+11 |
| 16 | 15.1 | 0.89 | 0.81 | 7.31E+11 |
| 17 | 15.5 | 0.87 | 0.86 | 1.04E+12 |
| 18 | 17.5 | 0.77 | 0.97 | 1.10E+12 |
| 19 | 19.1 | 0.71 | 0.94 | 1.06E+12 |
| 20 | 14.0 | 0.96 | 0.92 | 6.17E+11 |
| 21 | 18.6 | 0.73 | 1.15 | 1.33E+12 |
| 22 | 18.9 | 0.71 | 1.06 | 1.15E+12 |
| 23 | 14.8 | 0.91 | 1.01 | 1.07E+12 |
| 24 | 12.6 | 1.07 | 0.84 | 9.06E+11 |
| 25 | 11.6 | 1.16 | 0.96 | 1.01E+12 |
| 26 | 18.9 | 0.71 | 1.15 | 1.54E+12 |
| 27 | 20.2 | 0.67 | 1.06 | 1.49E+12 |
| 28 | 20.6 | 0.66 | 1.13 | 1.42E+12 |
| 29 | 18.6 | 0.73 | 1.13 | 1.61E+12 |
| 30 | 19.5 | 0.69 | 1.14 | 1.07E+12 |
| 31 | 13.9 | 0.97 | 0.84 | 4.03E+11 |
| 32 | 12.9 | 1.05 | 0.90 | 6.10E+11 |
| 33 | 16.6 | 0.81 | 1.00 | 9.35E+11 |
| 34 | 17.3 | 0.78 | 1.10 | 8.50E+11 |

### Example 4: Pilot Scale Production of AAV Vectors Using Continuous Transfection

This example describes 250 L scale production of an AAV vector using the methods and systems of the disclosure. As described in other examples, a continuous transfection process using a static in-line mixer and short controlled transfection cocktail incubation times yielded high titers and percentage of full capsids of an AAV vector at small scale. This example describes experiments to determine whether a similar process implemented with larger volumes of cells consistent with clinical drug supply or small-scale commercial manufacturing could yield similar results.

The overall experimental design was similar to that in Examples 2 and 3, and used the same types of plasmids, transfection reagent, media and cells. A static in-line mixing system similar to that described in Example 2 was constructed using larger components to accommodate the larger volume of transfection cocktail and cells. The tubing (Saint-Gobain C-flex) connecting reservoirs for containing the PEI and pDNA solution, T connector (serving as a static in-line mixer) and the bioreactor had ⅜ inch inner diameter and ⅝ inch outer. Most of the length of tube leading from the mixer to the bioreactor was coiled around one or more columns to enhance mixing effectiveness. The peristaltic pumps for pumping the solutions of PEI and pDNA out of their containers to the mixer and then to the bioreactor were calibrated to each other and set at half the flow rate calculated to result in the desired transfection cocktail incubation time and addition time. The containers of PEI and pDNA solutions were mounted on electronic scales so that small differences in pump rate could be detected and corrected to ensure equal amounts of both solutions were being combined.

For transfection, sufficient pDNA and PEI were respectively prepared in solution so that 0.75 μg pDNA would be added per 1×10⁶ viable cells and the PEI to pDNA mass ratio in transfection cocktail was 2.2:1. The mass ratios of the plasmids were 2.0 (helper):1.6 (rep/cap):1.0 (transgene), equivalent to molar ratios of 0.94:1.93:1.00, respectively. Media used to dilute stocks of PEI and pDNA was supplemented with Glutamax™ (ThemoFisher Scientific) to a final concentration of 10 mM and 0.2% Pluronic F-68. Because a larger volume of cells was required for these experiments, cells were expanded from a working cell bank through multiple stages, including growth in two shake flasks, a WAVE bioreactor, a 50 L single-use bioreactor and finally in a 250 L bioreactor (ThermoFisher 250 L 5:1 Aegis 5-14) with perfusion.

When the cells reached a target viable cell density of approximately 18×10⁶ cells/mL, perfusion was stopped and transfection started by pumping PEI and pDNA solutions into the system at equal rates to form transfection cocktail for between 30 and 90 seconds before being delivered to the cells in the bioreactor. After 3 hours, transfection was quenched by pumping in CDM4HEK293 media. Cells were then incubated for 72 hours with addition of fresh nutrient feed media as needed to permit AAV vector production, after which samples were taken, vector purified and assayed to determine titer (by qPCR either for ITR or transgene sequence) and to estimate proportion of full capsids (by absorbance ratio at 260 nm and 280 nm). Results are summarized in Table 4. Continuous transfection at 250 L pilot scale produced comparable amounts of vector to control bolus transfections, as determined by qPCR titer, and consistently produced vector with higher SEC UV260/UV280 values, suggesting continuous transfection at this scale results in a higher proportion of full capsids than bolus transfection.

TABLE 4

| Transfection | Incubation | Addition | Total Pump | Post Mixer | | Titer | Titer (vg/mL) | SEC |
|---|---|---|---|---|---|---|---|---|
| Method & | Time | Time | Rate | Tube Length | Reynolds | (vg/mL) | (Transgene | UV260/ |
| Experiment No. | (sec) | (min) | (L/min) | (feet) | Number | (ITR qPCR) | qPCR) | UV280 |
| Bolus 1 | | | | | | 1.88E+12 | Not tested | 0.94 |
| Bolus 2 | | | | | | 2.25E+12 | Not tested | 0.99 |
| Continuous 1 | 30 | 30 | 1.53 | 20 | 2870 | 1.29E+12 | Not tested | 1.25 |
| Continuous 2 | 60 | 16 | 2.88 | 74 | 5382 | 1.96E+12 | Not tested | 1.15 |
| Continuous 3 | 60 | 30 | 1.33 | 40 | 2870 | 1.76E+12 | Not tested | 1.19 |
| Continuous 4 | 90 | 30 | 1.53 | 59 | 2864 | 1.90E+12 | 7.65E+11 | Not tested |
| Continuous 5 | 90 | 30 | 1.53 | 59 | 2864 | 1.47E+12 | Not tested | 1.17 |
| Continuous 6 | 90 | 30 | 1.53 | 59 | 2864 | Not tested | 5.99E+11 | 1.06 |
| Continuous 7 | 90 | 30 | 1.53 | 59 | 2864 | Not tested | 2.49E+11 | Not tested |
| Continuous 8 | 90 | 30 | 1.53 | 59 | 2864 | Not tested | 1.02E+12 | 1.03 |

### Example 5: Large Scale Production of AAV Vectors Using Continuous Transfection

This example describes 2000 L scale production of an AAV vector using the methods and systems of the disclosure. As described in other examples, a continuous transfection process using a static in-line mixer and short controlled transfection cocktail incubation times yielded high titers and percentage of full capsids of an AAV vector at small scale and pilot scale. This example describes experiments to determine whether a similar process implemented with larger volumes of cells consistent with large scale commercial drug supply manufacturing could yield similar results.

The overall experimental design was similar to that in Examples 2, 3 and 4, and used the same types of plasmids, transfection reagent, media and cells. A static in-line mixing system similar to that described in Examples 2 and 3 was constructed using yet larger components to accommodate the larger volume of transfection cocktail and cells. In all experiments, the tubing connecting the T connector (serving as a static in-line mixer) with the bioreactor had 0.75 inch inner diameter and was 78 feet in length. In numbered experiments, two sets of mixing assemblies were utilized as shown schematically in FIG. 2 to achieve more rapid addition of the transfection cocktail to the bioreactor. Suspension-adapted HEK293 cells were grown in FreeStyle™ F17 medium (ThermoFisher Scientific) supplemented with 10 mM Glutamax™ (ThemoFisher Scientific) and 0.2% Pluronic F-68 from a frozen vial of a working cell bank and expanded through intermediate steps of shake flask, 10 L WAVE bag, 50 L WAVE bag, 200 L bioreactor and finally into a 2000 L single use bioreactor (Cytiva Xcellerex XDR 2000). In the final bioreactor, cells were perfused to remove spent media and add fresh, and grown to target viable cell density (VCD) of approximately 18×10⁶ cells/mL (although actual VCD varied somewhat depending on the experiment), and then continuously transfected with transfection cocktail.

When cells reached their target VCD, equal volumes of solutions containing PEI and pDNA were prepared to form a total volume of transfection cocktail 32.7% (wily) of the cell culture volume before transfection. After thawing, the specified amount of each plasmid stock was transferred to a single use mixer (SUM) containing the specified amount of F17 media supplemented with 10 mM Glutamax™ and 0.2% Pluronic F-68. In a separate SUM, the specified amount of a stock of fully depropionylated 40 KDa linear polyethylenimine (PEI) (1 mg/mL) was diluted in F17 media supplemented with 10 mM Glutamax™ and 0.2% Pluronic F-68 to serve as a transfection reagent. The contents of each SUM were slowly mixed for up to 15 minutes before and during transfection.

To start transfection, PEI and plasmid solutions were pumped at similar rates from the SUMs into tubes attached to the inlets of a T-connector serving as a static in-line mixer. Upon meeting at the intersection of the T-connector, the PH and plasmid solutions began mixing together to form transfection cocktail, which continued as the cocktail progressed down another longer tube between the outlet of the T-connector and the bioreactor containing the HEK293 cells. Portions of the tube leading from the T-connector to the bioreactor (incubation tube) were coiled to promote mixing of the PH and plasmid solutions. The length and diameter of the latter tube were chosen to achieve a certain cocktail incubation time from the T-connector to the bioreactor based on the pump rate. During addition of transfection cocktail, the bioreactor contents were agitated to distribute the cocktail among the cells. After all cocktail was added, transfection was quenched 3 hours later by pumping in CDM4HEK293 media. Cells were then incubated for 68-72 hours, after which AAV vectors isolated from cell samples were analyzed for titer and proportion of full capsids.

The conditions for fourteen different experiments are summarized in Table 5 and the results summarized in Table 6. AAV vector titer was determined using a quantitative PCR assay specific for transgene sequences and expressed as vector genomes per milliliter. Proportion of full versus empty capsids was estimated by measuring the UV absorbance ratio at 260 nm and 280 nm after purification by size exclusion chromatography (SEC UV260/UV280). The results were consistent with pilot scale (250 L) transfection experiments, which yielded an average vector titer of 6.29E+11 vg/mL and an average SEC UV260/UV280 value of 1.06.

TABLE 5

| Experiment No. | 1 | 2 | 3 | 4 | 5 | 6 | 7 |
|---|---|---|---|---|---|---|---|
| pDNA mass ratio | 2.0:1.6:1.0 | 2.0:1.6:1.0 | 2.0:1.7:1.0 | 2.0:1.6:1.0 | 2.0:1.6:1.0 | 2.0:1.5:1.0 | 2.0:1.6:1.0 |
| helper:rep/cap:transgene | | | | | | | |
| Mass of 3 pDNA | 15.6 | 15.4 | 15.7 | 15.4 | 15.7 | 15.8 | 16.2 |
| stock (1 mg | | | | | | | |
| pDNA/mL) mixed | | | | | | | |
| w/media (kg) | | | | | | | |
| Viable cell density | 17.5 | 16.7 | 17.7 | 18.2 | 22.5 | 16.4 | 16.0 |
| at transfection | | | | | | | |
| (×10⁶ cells/mL) | | | | | | | |
| pDNA per cell | 0.77 | 0.81 | 0.82 | 0.75 | 0.60 | 0.83 | 0.87 |
| (μg/10⁶ viable | | | | | | | |
| cells) | | | | | | | |
| Mass of PEI stock | 35.4 | 35.6 | 35.8 | 35.3 | 35.6 | 36.1 | 35.9 |
| (1 mg PEI/mL) | | | | | | | |
| mixed w/media | | | | | | | |
| (kg) | | | | | | | |
| PEI to pDNA mass | 2.3:1 | 2.3:1 | 2.3:1 | 2.3:1 | 2.3:1 | 2.3:1 | 2.2:1 |
| ratio | | | | | | | |
| Mass of mixture of | 200 | 198 | 198 | 198 | 198 | 198 | 198 |
| pDNA stock & | | | | | | | |
| media used in expt | | | | | | | |
| (kg) | | | | | | | |
| Mass of mixture of | 198 | 198 | 198 | 197 | 198 | 197 | 198 |
| PEI stock & media | | | | | | | |
| used in expt (kg) | | | | | | | |
| Mass of | 366 | 367 | 365 | 366 | 366 | 365 | 367 |
| transfection | | | | | | | |
| cocktail delivered | | | | | | | |
| to cells (kg) | | | | | | | |
| Post-transfection | 1480 | 1482 | 1480 | 1477 | 1482 | 1475-1500 | 1475-1500 |
| bioreactor | | | | | | | |
| contents mass (kg) | | | | | | | |
| Transfection | 91 | 90 | 101 | 98 | 96 | 98 | 133 |
| cocktail incubation | | | | | | | |
| time (seconds) | | | | | | | |
| Transfection | 41 | 41 | 45 | 44 | 43 | 44 | 60 |
| cocktail addition | | | | | | | |
| time (minutes) | | | | | | | |
| Transfection | 8.9 | 9.0 | 8.1 | 8.3 | 8.5 | 8.3 | 6.1 |
| cocktail addition | | | | | | | |
| flow rate (L/min) | | | | | | | |
| Post T-connector | 7 | 7 | 10 | coil 1 | 9 | 6-10 | 6-10 |
| tubing coil | | | | 8 | | | |
| diameter (inches) | | | | coil 2 | | | |
| | | | | 9 | | | |
| Reynolds Number | 4656 | 4656 | 4656 | 4656 | 4656 | 4656 | 3326 |
| (Re) | | | | | | | |
| Experiment No. | 8 | 9 | 10 | 11 | 12 | 13 | 14 |
| pDNA mass ratio | 2.0:1.6:1.0 | 2.0:1.6:1.0 | 2.0:1.6:1.0 | 2.0:1.6:1.0 | 2.0:1.6:1.0 | 1.4:1.5:1.0 | 2.0:1.6:1.0 |
| helper:rep/cap:transgene | | | | | | | |
| Mass of 3 pDNA | 16.6 | 16.4 | 15.9 | 16.4 | 16.4 | 16.8 | 16.4 |
| stock (1 mg | | | | | | | |
| pDNA/mL) mixed | | | | | | | |
| w/media (kg) | | | | | | | |
| Viable cell density | 18.4 | 17.0 | 19.2 | 16.6 | 18.1 | 17.0 | 19.2 |
| at transfection | | | | | | | |
| (×10⁶ cells/mL) | | | | | | | |
| pDNA per cell | 0.78 | 0.83 | 0.71 | 0.85 | 0.78 | 0.85 | 0.73 |
| (μg/10⁶ viable | | | | | | | |
| cells) | | | | | | | |
| Mass of PEI stock | 37.9 | 36.0 | 36.3 | 35.1 | 35.8 | 35.5 | 36.3 |
| (1 mg PEI/mL) | | | | | | | |
| mixed w/media | | | | | | | |
| (kg) | | | | | | | |
| PEI to pDNA mass | 2.3:1 | 2.2:1 | 2.3:1 | 2.1:1 | 2.2:1 | 2.1:1 | 2.2:1 |
| ratio | | | | | | | |
| Mass of mixture of | 196 | 198 | 198 | 198 | 198 | 197 | 198 |
| pDNA stock & | | | | | | | |
| media used in expt | | | | | | | |
| (kg) | | | | | | | |
| Mass of mixture of | 199 | 198 | 198 | 198 | 200 | 198 | 198 |
| PEI stock & media | | | | | | | |
| used in expt (kg) | | | | | | | |
| Mass of | 367 | 367 | 366 | 380 | 366 | 366 | 366 |
| transfection | | | | | | | |
| cocktail delivered | | | | | | | |
| to cells (kg) | | | | | | | |
| Post-transfection | 1475-1500 | 1475-1500 | 1475-1500 | 1475-1500 | 1475-1500 | 1475-1500 | 1475-1500 |
| bioreactor | | | | | | | |
| contents mass (kg) | | | | | | | |
| Transfection | 143 | 127 | 136 | 138 | 136 | 140 | 157 |
| cocktail incubation | | | | | | | |
| time (seconds) | | | | | | | |
| Transfection | 64 | 57 | 61 | 64 | 61 | 63 | 70 |
| cocktail addition | | | | | | | |
| time (minutes) | | | | | | | |
| Transfection | 5.7 | 6.4 | 6.0 | 5.9 | 6.0 | 5.8 | 5.2 |
| cocktail addition | | | | | | | |
| flow rate (L/min) | | | | | | | |
| Post T-connector | 6-10 | 6-10 | 6-10 | 6-10 | 6-10 | 6-10 | 6-10 |
| tubing coil | | | | | | | |
| diameter (inches) | | | | | | | |
| Reynolds Number | 3326 | 3326 | 3326 | 3326 | 3326 | 3326 | 3326 |
| (Re) | | | | | | | |

TABLE 6

| | Titer (Transgene | |
|---|---|---|
| Experiment No. | qPCR vg/mL) | SEC UV260/UV280 |
| 1 | 1.90E+11 | 1.10 |
| 2 | 4.55E+11 | 1.04 |
| 3 | 9.19E+11 | 1.09 |
| 4 | 2.19E+11 | 1.11 |
| 5 | 6.02E+11 | 1.05 |
| 6 | 3.92E+11 | 1.09 |
| 7 | 6.79E+11 | 1.08 |
| 8 | 7.64E+11 | 1.05 |
| 9 | 5.79E+11 | Not tested |
| 10 | 3.80E+11 | 1.02 |
| 11 | 4.59E+11 | 0.98 |
| 12 | 3.79E+11 | 1.07 |
| 13 | 1.62E+12 | Not tested |
| 14 | 1.12E+12 | Not tested |

### Example 6: Tubing Length Calculation to Achieve Target Incubation Time

This example describes an exemplary calculation of tubing length in a system of the disclosure at 1 L scale necessary to achieve transfection cocktail incubation time. In this example, solutions of PEI (transfection reagent) and plasmid DNA are contained in separate reservoirs and pumped by peristaltic pumps (one for each solution) through tubing leading to a static in-line mixer in the form of a tee connector, from which runs a third tube carrying PEI/pDNA transfection cocktail to a bioreactor containing the cells to be transfected. Based on certain defined variables, the length of the third tube is calculated to achieve a predetermined transfection cocktail incubation time.

In this example, the desired total transfection cocktail volume is 229 mL (115 mL PEI solution+115 mL pDNA solution); desired addition time is 30 min; desired incubation time is 90 sec (1.5 min); and the bore of the tube from the mixer to bioreactor is 3.175 mm (0.125 in). First, the system flow rate required to achieve the addition time is calculated. From the system flow rate, the pump rate for each of the two pumps (assuming 1:1 mixture of transfection reagent and plasmid DNA solutions) can also be calculated.

Total transfection cocktail volume/Addition time=System flow rate

229 mL/30 min=7.63 mL/min

Pump rate(per pump)=System flow rate/2

7.63 mL/min/2=3.82 mL/min per pump

Next, the volume per unit length (mL/cm) of the tube carrying the transfection cocktail to the bioreactor is calculated using the formula for the volume of a 1 cm long cylinder (3.14*r²*h).

(Tubing inner diameter/2)²*3.14*Height=Volume

(0.3175 cm/2)²*3.14*1 cm=0.08 mL/cm

Last, the length of tubing to achieve the desired incubation time from the tee-mixer to the bioreactor given the system flow rate and tubing bore can be calculated as follows.

(Incubation time*Flow rate)/Tubing volume per cm=Length

(1.5 min*7.63 mL/min)/0.08 mL/cm=145 cm

Thus, in the system described in this example, 145 cm of tubing with 3.175 bore connecting a mixer to a bioreactor would be needed for transfection cocktail to mix and incubate for 90 seconds before addition to cells given a system flow rate of 7.63 mL/min.

### Example 7: Effect of Calculated Reynolds Number on AAV Vector Potency

This experiment describes the effect of calculated Reynolds number (Re) associated with the flow of transfection cocktail between a static in-line mixer and a bioreactor on relative AAV vector potency at three different scales.

AAV vector containing a transgene to encode a mini-dystrophin was produced by transient triple transfection of HEK293 host cells in suspension culture at three different scales, L, 250 L and 2000 L. The three plasmids included the helper, rep/cap and mini-dystrophin transgene used in previous examples.

The 2000 L scale experiments are the same as those described in Example 5 and the 250 L scale experiments are the same as those described in Example 4. The 10 L scale experiments used similar reagents and methods as the larger scale experiments, as well as a system for transfection using a static in-line mixer, although at commensurately smaller scale. Based on the pump rate and other characteristics of the systems used for these experiments, Reynolds number for each experiment was calculated and correlated to the potency of the AAV vector produced from each experiment. Vector potency was determined by measuring the amount of mini-dystrophin protein produced in vitro by differentiated myotubes transduced with the vectors. Additionally, the percentage of capsids that were not completely filled with DNA (partially filled capsids) was estimated using a capillary gel electrophoresis method. In general, a higher percentage of completely full capsids is considered desirable. Reynold's number (Re) is calculated as Re=ρνD/μ, where ρ is the density of the transfection cocktail (assumed to be 997 kg/m 3), ν is the linear velocity of the transfection cocktail as it flows through the tubing (m/s), D is the inner diameter of the tube (m), and μ is the viscosity (assumed to be 8.90×10⁻⁴ Pa*s).

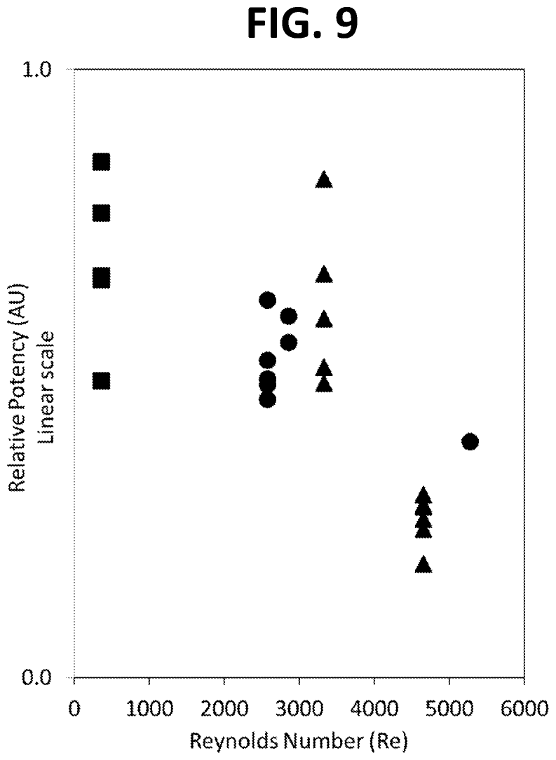

The results are shown in Table 7. As can be seen, higher Re values associated with turbulent flow (Re>4000) resulted in lower vector potency, whereas lower Re values associated with non-turbulent flow (Re<4000) resulted in higher vector potency. This relationship was consistent at both 250 L and 2000 L scales of production. A graphical representation of the same data indicates that relative vector potency is negatively correlated with Reynold's number (FIG. 9). At the larger 2000 L scale, there was also a reduction in the percentage of partially filled capsids for vectors produced in experiments with lower Re values associated with non-turbulent flow. These results suggest that continuous flow transfection systems for AAV vector production can be designed to avoid turbulent flow of transfection cocktail (for example, so that Re values are <4000) to maximize potential vector potency and/or percentage of full capsids in resulting drug substance. The relationship between potency and incubation time (time for transfection cocktail to transit length of tube from static in-line mixer to bioreactor) was also examined, but no correlation was found (data not shown).

TABLE 7

| | Transfection Cocktail | | | |
|---|---|---|---|---|
| Experiment No. | Incubation Time | Calculated Reynolds | DS Relative Potency | DS Partially Full |
| (Scale) | (Secs) | Number | (%) | Capsids (%) |
| Experiment 1 (10 L) | 90 | 369 | 196 | 34.25 |
| Experiment 2 (10 L) | 90 | 369 | 229 | 44.22 |
| Experiment 3 (10 L) | 90 | 369 | 254 | 38.7 |
| Experiment 4 (10 L) | 90 | 369 | 198 | 43.18 |
| Experiment 5 (10 L) | 135 | 369 | 146 | 43.62 |
| Experiment 1 (250 L) | 30 | 2870 | 165 | 52.21 |
| Experiment 2 (250 L) | 60 | 5282 | 116 | 61.19 |
| Experiment 3 (250 L) | 60 | 2870 | 178 | 57.66 |
| Experiment 4 (250 L) | 90 | 2582 | 186 | 52.72 |
| Experiment 5 (250 L) | 90 | 2582 | 156 | 62.97 |
| Experiment 6 (250 L) | 90 | 2582 | 137 | 55.66 |
| Experiment 7 (250 L) | 90 | 2582 | 147 | 57.67 |
| Experiment 8 (250 L) | 90 | 2582 | 144 | 46.67 |
| Experiment 1 (2000 L) | 97 | 4656 | 73 | 70.32 |
| Experiment 2 (2000 L) | 97 | 4656 | 56 | 66.07 |
| Experiment 3 (2000 L) | 97 | 4656 | 78 | 60.12 |
| Experiment 4 (2000 L) | 97 | 4656 | 84 | 58.11 |
| Experiment 5 (2000 L) | 97 | 4656 | 91 | 62.05 |
| Experiment 6 (2000 L) | 97 | 4656 | 85 | 62.27 |
| Experiment 7 (2000 L) | 135 | 3326 | 199 | 32.61 |
| Experiment 8 (2000 L) | 135 | 3326 | 145 | 39.61 |

What is claimed is:

1. A method for transfecting host cells with nucleic acid, comprising continuously forming and delivering a transfection cocktail comprising a transfection reagent and a nucleic acid to cells in culture.

2. The method of claim 1, wherein transfection cocktail is formed by mixing separate solutions, each respectively comprising the transfection reagent and the nucleic acid.

3. The method of any one of the preceding claims, wherein the transfection reagent is a cationic polymer.

4. The method of any one of the preceding claims, wherein the transfection reagent is a polyethylenimine (PEI).

5. The method of any one of the preceding claims, wherein the nucleic acid is DNA.

6. The method of any one of the preceding claims, wherein the DNA is plasmid DNA (pDNA) or bacmid DNA.

7. The method of any one of claims 2-6, wherein the transfection reagent and nucleic acid solutions comprise cell media.

8. The method of any one of the preceding claims, wherein the transfection cocktail, once formed, is delivered to the cells in less than or about 25, 15, 10, 5, or 4 minutes, or less than or about 180, 150, 135, 120, 90, 60, 45, 30, or 15 seconds.

9. The method of any one of the preceding claims, wherein substantially the entire volume of transfection cocktail is delivered to the cells in less than or about 120, 90, 60, 45, 40, 30, 20, 10, or 5 minutes.

10. The method of claim 5 or 6, wherein the transfection cocktail comprises sufficient DNA such that cells are transfected with at least or about 0.25, 0.50, 0.75, 1.00, 1.25, 1.50, 1.75, 2.00 μg DNA/10⁶ viable cells, or ranges from about 0.50 to 1.00 μg DNA/10⁶ viable cells.

11. The method of claim 10, wherein the transfection cocktail comprises sufficient PEI such that the mass ratio of PEI to DNA in the transfection cocktail is at least or about 0.5, 1.0, 1.5, 2.0, 2.5, or 3.0, or ranges from about 1.2 to 3.2, or is about 2.2.

12. The method of any of the preceding claims, wherein transfection cocktail is delivered to the cells at a viable cell (vc) density of at least or about 10×10⁶, 15×10⁶, 20×10⁶, 25×10⁶, 30×10⁶, 35×10⁶, 40×10⁶, 45×10⁶, or 50×10⁶ vc/mL culture volume, or ranges from about 10×10⁶ to 30×10⁶ vc/mL, about 15×10⁶ to 25×10⁶ vc/mL, or about 16×10⁶ to 24×10⁶ vc/mL.

13. The method of any of the preceding claims, wherein the volume of transfection cocktail delivered to the cells is at least or about 10%, 20%, 25%, 30%, 35%, 40%, or 45% of the volume of the cell culture before transfection, or ranges from about 25% to 45%, or about 30% to 40%.

14. The method of any one of claims 2 to 13, wherein the transfection reagent solution comprises 10% to 30% PEI (w/v) and the nucleic acid solution comprises 5% to 15% DNA (w/v).

15. The method of any one of the preceding claims, wherein the cells are mammalian cells or insect cells.

16. The method of any one of the preceding claims, wherein the cells are BHK cells, CHO cells, HEK293 cells, or HeLa cells.

17. The method of any one of the preceding claims, wherein the nucleic acid comprises a sequence encoding a biological product, or a component thereof.

18. The method of claim 17, wherein the nucleic acid further comprises a transcription control region operatively linked to said sequence encoding the biological product, or component thereof.

19. The method of claim 18, wherein the transcription control region comprises a promoter and optionally an enhancer.

20. The method of claim 17, wherein the biological product comprises a protein or a component of a recombinant viral vector.

21. The method of claim 20, wherein the recombinant viral vector an adenoviral vector, an adeno-associated viral (AAV) vector, a lentiviral vector, or a retroviral vector.

22. The method of any one of the preceding claims, wherein the nucleic acid comprises a sequence element selected from the group consisting of: a gene for a viral helper factor, a AAV rep gene, an AAV cap gene, and a vector genome comprising a transgene capable of being packaged in an AAV capsid.

23. The method of any one of the preceding claims, wherein before transfection, the volume of the cells in culture is at least 100 L, 500 L, or 1000 L.

24. The method of any one of the preceding claims, further comprising incubating the cells after transfection is complete and isolating a biological product made by the cells as a result of transfection.

25. The method of claim 24, wherein the biological product is a recombinant AAV vector, wherein the method is effective to produce recombinant AAV vectors having a titer of at least or about 1×10¹⁰, 5×10¹⁰, 1×10¹¹, 5×10¹¹, 1×10¹², 5×10¹², or 1×10¹³ vector genomes per milliliter (vg/mL) of cell suspension after transfection, and wherein the method is effective to produce recombinant AAV vectors having, after purification by size exclusion chromatography, a UV260/UV280 absorbance ratio of at least or about 0.4, 0.5, 0.6, 0.7, 0.8, 0.9, 1.0, 1.1, 1.2, 1.3, 1.4, 1.5, 1.6, 1.7, or 1.8.

26. A biological product produced by the method of claim 24.

27. The biological product of claim 26, wherein said biological product is a recombinant AAV vector.

28. A system for transfecting cells, comprising (i) means for separately containing transfection reagent and nucleic acid solutions, (ii) means for pumping said solutions from their respective containment means, (iii) means for mixing said solutions, forming a transfection cocktail, (iv) means for containing cells to be transfected, and (v) means for fluid communication from said solution containment means to said mixing means, and therefrom to said cell containment means.

29. The system of claim 28, wherein said mixing means comprises a static in-line mixer.

30. The system of any one of claim 28 or 29, wherein said system is configured such that flow of transfection cocktail within said system is not turbulent.

31. The system of any one of claims 28 to 30, wherein said system is configured such that Reynolds number Re associated with flow of transfection cocktail within said system does not exceed a value of 3500.

32. The system of any one of claims 28 to 30, wherein said system is configured such that Reynolds number Re associated with flow of transfection cocktail within said system does not exceed a value of 4000.
